SEQUENCE LISTING (59 sequences)

SEQ ID NO: 1 - protein, 5 aa, Artificial Sequence
  Synthetic Construct
GYWMQ

SEQ ID NO: 2 - protein, 17 aa, Artificial Sequence
  Synthetic Construct
AIYPGDGDTRYSQKFKG

SEQ ID NO: 3 - protein, 9 aa, Artificial Sequence
  Synthetic Construct
RDYGAWFAY

SEQ ID NO: 4 - protein, 11 aa, Artificial Sequence
  Synthetic Construct
KASQDINSYLS

SEQ ID NO: 5 - protein, 7 aa, Artificial Sequence
  Synthetic Construct
RANRLVD

SEQ ID NO: 6 - protein, 9 aa, Artificial Sequence
  Synthetic Construct
LQYDEFPYT

SEQ ID NO: 7 - protein, 118 aa, Artificial Sequence
  Synthetic Construct
QVQFQQSGAELARPGASVKLSCKASGYTFTGYWMQWVKQRPGQGLEWIGAIYPGDGDTRYSQKFKGKVTLTADKSSSTAYMQLSSLASEDSAVYYCARRDYGAWFAYWGQGTLVTVSA

SEQ ID NO: 8 - protein, 107 aa, Artificial Sequence
  Synthetic Construct
DIKVTQSPSSMYASLGERVTITCKASQDINSYLSWFQQKPGKSPKTLIYRANRLVDGVPSRFSGSGSGQDYSLTISSLDYEDMGIYYCLQYDEFPYTFGGGTKLEIK

SEQ ID NO: 9 - protein, 5 aa, Artificial Sequence
  Synthetic Construct
SSWVE

SEQ ID NO: 10 - protein, 17 aa, Artificial Sequence
  Synthetic Construct
EIFPGSGSTHYNEKFKG

SEQ ID NO: 11 - protein, 9 aa, Artificial Sequence
  Synthetic Construct
RPPGWFFDV

SEQ ID NO: 12 - protein, 11 aa, Artificial Sequence
  Synthetic Construct
KASQDVSTDVA

SEQ ID NO: 13 - protein, 7 aa, Artificial Sequence
  Synthetic Construct
SASYRYT

SEQ ID NO: 14 - protein, 9 aa, Artificial Sequence
  Synthetic Construct
QQHYSTPWT

SEQ ID NO: 15 - protein, 118 aa, Artificial Sequence
  Synthetic Construct
QVQLQQSGAELMKPGASVKISCKATGYTFSSSWVEWVRQRPGHGLEWIGEIFPGSGSTHYNEKFKGKATFTADTSSNTAYLQLSSLTSDDSAVYYCARRPPGWFFDVWGAGTTVTVSS

SEQ ID NO: 16 - protein, 107 aa, Artificial Sequence
  Synthetic Construct
DIVMTQSHKFMSTSVGDRVSITCKASQDVSTDVAWYQQKPGQSPKLLIYSASYRYTGVPDRFTGSGSGTDFTFTISSVQAEDLAVYYCQQHYSTPWTFGGGTKLEIK

SEQ ID NO: 17 - protein, 5 aa, Artificial Sequence
  Synthetic Construct
SSWIE

SEQ ID NO: 18 - protein, 17 aa, Artificial Sequence
  Synthetic Construct
EIFPGSGSLNFNEKFKG

SEQ ID NO: 19 - protein, 9 aa, Artificial Sequence
  Synthetic Construct
RPPGWFFDV

SEQ ID NO: 20 - protein, 11 aa, Artificial Sequence
  Synthetic Construct
KASQDVTTDVA

SEQ ID NO: 21 - protein, 7 aa, Artificial Sequence
  Synthetic Construct
SASYRYT

SEQ ID NO: 22 - protein, 9 aa, Artificial Sequence
  Synthetic Construct
QQHYSSPWT

SEQ ID NO: 23 - protein, 118 aa, Artificial Sequence
  Synthetic Construct
QVQLQQSGAELMKPGASVKISCKATGYTFSSSWIEWVKQRPGHGLEWIGEIFPGSGSLNFNEKFKGKATFTADTSSNTAYLQLSSLTSDDSAVYYCARRPPGWFFDVWGAGTTVTVSS

SEQ ID NO: 24 - protein, 107 aa, Artificial Sequence
  Synthetic Construct
DIVMTQSHKFMSTSVGDRVSITCKASQDVTTDVAWYQQKPGQSPKLLIYSASYRYTGVPDRFTGSGSGTDFTFTISSVQAEDLTVYYCQQHYSSPWTFGGGTKLEIK

SEQ ID NO: 25 - protein, 5 aa, Artificial Sequence
  Synthetic Construct
SCWIE

SEQ ID NO: 26 - protein, 17 aa, Artificial Sequence
  Synthetic Construct
EILPGSDYTNYNEKFKD

SEQ ID NO: 27 - protein, 9 aa, Artificial Sequence
  Synthetic Construct
RPPGWYFDV

SEQ ID NO: 28 - protein, 11 aa, Artificial Sequence
  Synthetic Construct
KASQDVSTDIA

SEQ ID NO: 29 - protein, 7 aa, Artificial Sequence
  Synthetic Construct
SASYRFT

SEQ ID NO: 30 - protein, 9 aa, Artificial Sequence
  Synthetic Construct
QHQYSTPWT

SEQ ID NO: 31 - protein, 118 aa, Artificial Sequence
  Synthetic Construct
QVQLQQSGAELMKPGASVKISCTATGYTFSSCWIEWVKQRPGHGLEWLGEILPGSDYTNYNEKFKDKATFTADTSSDTAYMQLSSLTSEDSAVYYCARRPPGWYFDVWGAGTAVTVSS

SEQ ID NO: 32 - protein, 107 aa, Artificial Sequence
  Synthetic Construct
DIVMTQSHKFMSTSVGDRVSITCKASQDVSTDIAWYQQKPGQSPKLLIYSASYRFTGVPDRFTGSGSGTDFTFTISSVQAEDLAVYYCQHQYSTPWTFGGGTKLDIK

SEQ ID NO: 33 - protein, 5 aa, Artificial Sequence
  Synthetic Construct
DTFIH

SEQ ID NO: 34 - protein, 17 aa, Artificial Sequence
  Synthetic Construct
RIDPANGNSKYDPKFQG

SEQ ID NO: 35 - protein, 10 aa, Artificial Sequence
  Synthetic Construct
WPSNWEAMDY

SEQ ID NO: 36 - protein, 10 aa, Artificial Sequence
  Synthetic Construct
SASSSVSYMY

SEQ ID NO: 37 - protein, 7 aa, Artificial Sequence
  Synthetic Construct
DTSNLAS

SEQ ID NO: 38 - protein, 9 aa, Artificial Sequence
  Synthetic Construct
QQWISYPLT

SEQ ID NO: 39 - protein, 119 aa, Artificial Sequence
  Synthetic Construct
EVQLQQSGSELVKAGASVKLSCTASGFKIKDTFIHWVKQRPEQGLDWIGRIDPANGNSKYDPKFQGKATITADTSSNTAYLQLSSLTSEDTAVYYCARWPSNWEAMDYWGQGTSVTVSS

SEQ ID NO: 40 - protein, 106 aa, Artificial Sequence
  Synthetic Construct
QIVLTQSPAIMSASPGEKVTMTCSASSSVSYMYWYQQKPGSSPRLLIYDTSNLASGVPFRFSGSGSGTSYSLTISRMEAEDAATYYCQQWISYPLTFGTGTKLELK

SEQ ID NO: 41 - protein, 5 aa, Artificial Sequence
  Synthetic Construct
  VARIANT 4: Xaa = V or I
SSWXE

SEQ ID NO: 42 - protein, 17 aa, Artificial Sequence
  Synthetic Construct
  VARIANT 9: Xaa = T or L
  VARIANT 10: Xaa = H or N
  VARIANT 11: Xaa = Y or F
EIFPGSGSXXXNEKFKG

SEQ ID NO: 43 - protein, 5 aa, Artificial Sequence
  Synthetic Construct
  VARIANT 2: Xaa = S or C
  VARIANT 4: Xaa = V or I
SXWXE

SEQ ID NO: 44 - protein, 17 aa, Artificial Sequence
  Synthetic Construct
  VARIANT 3: Xaa = F or L
  VARIANT 7: Xaa = G or D
  VARIANT 8: Xaa = Y or S
  VARIANT 10: Xaa = H or N
  VARIANT 17: Xaa = G or D
EIXPGSXXTXYNEKFKX

SEQ ID NO: 45 - protein, 9 aa, Artificial Sequence
  Synthetic Construct
  VARIANT 6: Xaa = F or Y
RPPGWXFDV

SEQ ID NO: 46 - protein, 11 aa, Artificial Sequence
  Synthetic Construct
  VARIANT 7: Xaa = S or T
KASQDVXTDVA

SEQ ID NO: 47 - protein, 9 aa, Artificial Sequence
  Synthetic Construct
  VARIANT 6: Xaa = S or T
QQHYSXPWT

SEQ ID NO: 48 - protein, 11 aa, Artificial Sequence
  Synthetic Construct
  VARIANT 7: Xaa = S or T
  VARIANT 10: Xaa = V or I
KASQDVXTDXA

SEQ ID NO: 49 - protein, 7 aa, Artificial Sequence
  Synthetic Construct
  VARIANT 6: Xaa = Y or F
SASYRXT

SEQ ID NO: 50 - protein, 9 aa, Artificial Sequence
  Synthetic Construct
  VARIANT 2, 3: Xaa = Q or H
  VARIANT 6: Xaa = S or T
QXXYSXPWT

SEQ ID NO: 51 - protein, 17 aa, Artificial Sequence
  Synthetic Construct
  VARIANT (9)..(11): Xaa = THY or LNF
EIFPGSGSXXXNEKFKG

SEQ ID NO: 52 - protein, 17 aa, Artificial Sequence
  Synthetic Construct
  VARIANT 3: Xaa = F or L
  VARIANT (7)..(8): Xaa = DY or GS
  VARIANT 10: Xaa = H or N
  VARIANT 17: Xaa = G or D
EIXPGSXXTXYNEKFKX

SEQ ID NO: 53 - protein, 9 aa, Artificial Sequence
  Synthetic Construct
  VARIANT (2)..(3): Xaa = QH or HQ
  VARIANT 6: Xaa = S or T
QXXYSXPWT

SEQ ID NO: 54 - protein, 11 aa, Artificial Sequence
  Synthetic Construct
  VARIANT 10: Xaa = V or I
KASQDVSTDXA

SEQ ID NO: 55 - protein, 7 aa, Artificial Sequence
  Synthetic Construct
  VARIANT 6: Xaa = Y or F
SASYRXT

SEQ ID NO: 56 - protein, 9 aa, Artificial Sequence
  Synthetic Construct
  VARIANT 2, 3: Xaa = Q or H
QXXYSTPWT

SEQ ID NO: 57 - protein, 9 aa, Artificial Sequence
  Synthetic Construct
  VARIANT (2)..(3): Xaa = QH or HQ
QXXYSTPWT

SEQ ID NO: 58 - protein, 17 aa, Artificial Sequence
  Synthetic Construct
  VARIANT 3: Xaa = F or L
  VARIANT (7)..(11): Xaa = GSTHY, GSLNF, or DYTNY
  VARIANT 17: Xaa = F or L
EIXPGSXXXXXNEKFKX

SEQ ID NO: 59 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

(19) United States
(12) Patent Application Publication
(10) Pub. No.: US 2024/0141048 A1
(43) Pub. Date: May 2, 2024

(54) ANTI-VISTA CONSTRUCTS AND USES THEREOF

(71) Applicant: DYNAMICURE BIOTECHNOLOGY LLC

(72) Inventors: Zirong CHEN, Saugus, MA (US); Jian LI, Chestnut Hill, MA (US); Angela NORTON, Reading, MA (US); Shuo WANG, Lexington, MA (US); Lihua WU, Cambridge, MA (US); Zhinan XIA, Waltham, MA (US)

(21) Appl. No.: 18/280,458

(22) PCT Filed: Mar. 4, 2022
(86) PCT No.: PCT/US2022/070988

§ 371 Date: Sep. 5, 2023

Related U.S. Application Data
(60) Provisional application No. 63/157,182, filed on Mar. 5, 2021.

(51) Int. Cl. C07K 16/28; A61P 37/06; A61K 39/00

(52) U.S. Cl. CPC C07K 16/2827; A61P 37/06; A61K 2039/505; C07K 2317/33; C07K 2317/71; C07K 2317/73; C07K 2317/75; C07K 2317/92; C07K 2317/94

(57) ABSTRACT

The present application provides anti-VISTA constructs that bind to VISTA (e.g., anti-VISTA antibodies), nucleic acid molecules encoding an amino acid sequence of the anti-VISTA, vectors comprising the nucleic acid molecules, host cells containing the vectors, methods of preparing the anti-VISTA construct, pharmaceutical compositions containing the anti-VISTA construct, and methods of using the anti-VISTA construct or compositions.

### CROSS-REFERENCE TO RELATED APPLICATION

This application claims priority to U.S. provisional application 63/157,182, filed on Mar. 5, 2021, the content of which is incorporated by reference in its entirety for all purposes.

### TECHNICAL FIELD

The present disclosure relates to anti-VISTA constructs (such as anti-VISTA antibodies) and the uses thereof.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 193852000440SEQLIST.TXT, date recorded: Mar. 2, 2022, size: 24,739 bytes).

### BACKGROUND OF THE APPLICATION

VISTA (also known as programmed death-1 homologue, PD-1H, VSIR, Dies1, DD1α, Gi24) is a cell surface inhibitory molecule of the B7/CD28 gene family expressed on T cells and myeloid cells. VISTA can function as an inhibitory ligand on antigen presenting cells (APCs) and regulate T cell responses through an unknown receptor. In addition, VISTA can also function as an inhibitory receptor on T cells. For example, agonist VISTA monoclonal antibody (mAb) dramatically regulates antigen-specific CD4+ T cell responses and protects mice from graft-versus-host disease (GVHD) and experimental hepatitis. Mice deficient in VISTA on C57BL/6 background (B6 PD-1H KO) are more susceptible to autoimmune induction such as experimental autoimmune encephalomyelitis and systemic lupus when backcrossed to a lupus-prone strain. VISTA has been shown to be involved in peripheral immune tolerance and negatively regulates T cell activation. See e.g., Sci Transl Med. 2019 Dec 11;11(522).

The disclosures of all publications, patents, patent applications and published patent applications referred to herein are hereby incorporated herein by reference in their entirety.

### BRIEF SUMMARY OF THE APPLICATION

The present application in one aspect provides an anti-VISTA construct comprising an antibody moiety comprising a heavy chain variable region (VH) and a light chain variable region (VL), wherein the antibody moiety competes for a binding epitope of VISTA with an antibody or antibody fragment comprising a second heavy chain variable region (VH-2) and a second light chain variable region (VL-2), wherein:

a) the VH-2 comprising the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the VL-2 comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6;

b) the VH-2 comprises the HC-CDR1 comprising the amino acid sequence of SEQ ID NO: 9, the HC-CDR2 comprising the amino acid sequence of SEQ ID NO: 10, and the HC-CDR3 comprising the amino acid sequence of SEQ ID NO: 11, and the VL-2 comprises the LC-CDR1 comprising the amino acid sequence of SEQ ID NO: 12, the LC-CDR2 comprising the amino acid sequence of SEQ ID NO: 13, and the LC-CDR3 comprising the amino acid sequence of SEQ ID NO: 14;

c) the VH-2 comprises the HC-CDR1 comprising the amino acid sequence of SEQ ID NO: 17, the HC-CDR2 comprising the amino acid sequence of SEQ ID NO: 18, and the HC-CDR3 comprising the amino acid sequence of SEQ ID NO: 19, and the VL-2 comprises the LC-CDR1 comprising the amino acid sequence of SEQ ID NO: 20, the LC-CDR2 comprising the amino acid sequence of SEQ ID NO: 21, and the LC-CDR3 comprising the amino acid sequence of SEQ ID NO: 22;

d) the VH-2 comprises the HC-CDR1 comprising the amino acid sequence of SEQ ID NO: 25, the HC-CDR2 comprising the amino acid sequence of SEQ ID NO: 26, and the HC-CDR3 comprising the amino acid sequence of SEQ ID NO: 27, and the VL-2 comprises the LC-CDR1 comprising the amino acid sequence of SEQ ID NO: 28, the LC-CDR2 comprising the amino acid sequence of SEQ ID NO: 29, and the LC-CDR3 comprising the amino acid sequence of SEQ ID NO: 30; or

e) the VH-2 comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the VL-2 comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38.

In some embodiments, the VH comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising 5, 4, 3, 2, or 1 amino acid substitutions in the HC-CDRs; and the VL comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising 5, 4, 3, 2, or 1 amino acid substitutions in the LC-CDRs. In some embodiments, the VH comprises i) the HC-CDR1 comprising the amino acid sequence of SEQ ID NO: 9, ii) the HC-CDR2 comprising the amino acid sequence of SEQ ID NO: 10, and iii) the HC-CDR3 comprising the amino acid sequence of SEQ ID NO: 11, or a variant thereof comprising 5, 4, 3, 2, or 1 amino acid substitutions in the HC-CDRs; and the VL comprises i) the LC-CDR1 comprising the amino acid sequence of SEQ ID NO: 12, ii) the LC-CDR2 comprising the amino acid sequence of SEQ ID NO: 13, and iii) the LC-CDR3 comprising the amino acid sequence of SEQ ID NO: 14, or a variant thereof comprising 5, 4, 3, 2, or 1 amino acid substitutions in the LC-CDRs. In some embodiments, the VH comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, or a variant thereof comprising 5, 4, 3, 2, or 1 amino acid substitutions in the HC-CDRs; and the VL comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22, or a variant thereof comprising 5, 4, 3, 2, or 1 amino acid substitutions in the LC-CDRs. In some embodiments, the VH comprises i) the HC-CDR1 comprising the amino acid sequence of SEQ ID NO: 25, ii) the HC-CDR2 comprising the amino acid sequence of SEQ ID NO: 26, and iii) the HC-CDR3 comprising the amino acid sequence of SEQ ID NO: 27, or a variant thereof comprising 5, 4, 3, 2, or 1 amino acid substitutions in the HC-CDRs; and the VL comprises i) the LC-CDR1 comprising the amino acid sequence of SEQ ID NO: 28, ii) the LC-CDR2 comprising the amino acid sequence of SEQ ID NO: 29, and iii) the LC-CDR3 comprising the amino acid sequence of SEQ ID NO: 30, or a variant thereof comprising 5, 4, 3, 2, or 1 amino acid substitutions in the LC-CDRs. In some embodiments, the VH comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising 5, 4, 3, 2, or 1 amino acid substitutions in the HC-CDRs; and the VL comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising 5, 4, 3, 2, or 1 amino acid substitutions in the LC-CDRs. In some embodiments, the VH comprises i) the HC-CDR1 comprising the amino acid sequence of SEQ ID NO: 41, ii) the HC-CDR2 comprising the amino acid sequence of SEQ ID NO: 42 or 51, and iii) the HC-CDR3 comprising the amino acid sequence of SEQ ID NO: 11; and the VL comprises i) the LC-CDR1 comprising the amino acid sequence of SEQ ID NO: 46, ii) the LC-CDR2 comprising the amino acid sequence of SEQ ID NO: 13, and iii) the LC-CDR3 comprising the amino acid sequence of SEQ ID NO: 47. In some embodiments, the VH comprises i) the HC-CDR1 comprising the amino acid sequence of SEQ ID NO: 43, ii) the HC-CDR2 comprising the amino acid sequence of SEQ ID NO: 44 or 52, and iii) the HC-CDR3 comprising the amino acid sequence of SEQ ID NO: 45; and the VL comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 56 or 57. In some embodiments, the VH comprises i) the HC-CDR1 comprising the amino acid sequence of SEQ ID NO: 41 or 43, ii) the HC-CDR2 comprising the amino acid sequence of any of SEQ ID NO:58, and iii) the HC-CDR3 comprising the amino acid sequence of SEQ ID NO: 11 or 45; and the VL comprises i) the LC-CDR1 comprising the amino acid sequence of SEQ ID NO: 48, ii) the LC-CDR2 comprising the amino acid sequence of SEQ ID NO: 49, and iii) the LC-CDR3 comprising the amino acid sequence of SEQ ID NO: 50 or 53.

The present application in another aspect provides an anti-VISTA construct comprising an antibody moiety that specifically binds to VISTA, comprising:

a) a HC-CDR1, a HC-CDR2, and a HC-CDR3, respectively comprising the amino acid sequences of a CDR1, a CDR2, and a CDR3 within a VH chain region having the sequence set forth in SEQ ID NO: 7, and a LC-CDR1, a LC-CDR2, and a LC-CDR3, respectively comprising the amino acid sequences of a CDR1, a CDR2, and a CDR3 within a VL chain region having the sequence set forth in SEQ ID NO: 8;

b) a HC-CDR1, a HC-CDR2, and a HC-CDR3, respectively comprising the amino acid sequences of a CDR1, a CDR2, and a CDR3 within a VH chain region having the sequence set forth in SEQ ID NO: 15, and a LC-CDR1, a LC-CDR2, and a LC-CDR3, respectively comprising the amino acid sequences of a CDR1, a CDR2, and a CDR3 within a VL chain region having the sequence set forth in SEQ ID NO: 16;

c) a HC-CDR1, a HC-CDR2, and a HC-CDR3, respectively comprising the amino acid sequences of a CDR1, a CDR2, and a CDR3 within a VH chain region having the sequence set forth in SEQ ID NO: 23, and a LC-CDR1, a LC-CDR2, and a LC-CDR3, respectively comprising the amino acid sequences of a CDR1, a CDR2, and a CDR3 within a VL chain region having the sequence set forth in SEQ ID NO: 24;

d) a HC-CDR1, a HC-CDR2, and a HC-CDR3, respectively comprising the amino acid sequences of a CDR1, a CDR2, and a CDR3 within a VH chain region having the sequence set forth in SEQ ID NO: 31, and a LC-CDR1, a LC-CDR2, and a LC-CDR3, respectively comprising the amino acid sequences of a CDR1, a CDR2, and a CDR3 within a VL chain region having the sequence set forth in SEQ ID NO: 32; or

e) a HC-CDR1, a HC-CDR2, and a HC-CDR3, respectively comprising the amino acid sequences of a CDR1, a CDR2, and a CDR3 within a VH chain region having the sequence set forth in SEQ ID NO: 39, and a LC-CDR1, a LC-CDR2, and a LC-CDR3, respectively comprising the amino acid sequences of a CDR1, a CDR2, and a CDR3 within a VL chain region having the sequence set forth in SEQ ID NO: 40.

In some embodiments according to any of the anti-VISTA constructs described above, the VH comprises an amino acid sequence of any one of SEQ ID NOs: 7, 15, 23, 31, and 39, or a variant comprising an amino acid sequence having at least about 80% sequence identity; and/or wherein the VL comprises an amino acid sequence of any one of SEQ ID NOs: 8, 16, 24, 32 and 40, or a variant comprising an amino acid sequence having at least about 80% sequence identity. In some embodiments, the VH comprises an amino acid sequence of SEQ ID NO: 7, or a variant comprising an amino acid sequence having at least about 80% sequence identity; and the VL comprises an amino acid sequence of SEQ ID NO: 8, or a variant comprising an amino acid sequence having at least about 80% sequence identity. In some embodiments, the VH comprises an amino acid sequence of SEQ ID NO: 15, or a variant comprising an amino acid sequence having at least about 80% sequence identity; and the VL comprises an amino acid sequence of SEQ ID NO: 16, or a variant comprising an amino acid sequence having at least about 80% sequence identity. In some embodiments, the VH comprises an amino acid sequence of SEQ ID NO: 23, or a variant comprising an amino acid sequence having at least about 80% sequence identity; and the VL comprises an amino acid sequence of SEQ ID NO: 24, or a variant comprising an amino acid sequence having at least about 80% sequence identity. In some embodiments, the VH comprises an amino acid sequence of SEQ ID NO: 31, or a variant comprising an amino acid sequence having at least about 80% sequence identity; and the VL comprises an amino acid sequence of SEQ ID NO: 32, or a variant comprising an amino acid sequence having at least about 80% sequence identity. In some embodiments, the VH comprises an amino acid sequence of SEQ ID NO: 39, or a variant comprising an amino acid sequence having at least about 80% sequence identity; and the VL comprises an amino acid sequence of SEQ ID NO: 40, or a variant comprising an amino acid sequence having at least about 80% sequence identity.

In some embodiments according to any of the anti-VISTA constructs described above, the antibody moiety is an antibody or antigen-binding fragment thereof selected from the group consisting of a full-length antibody, a bispecific antibody, a single-chain Fv (scFv) fragment, a Fab fragment, a Fab' fragment, a F(ab')2, an Fv fragment, a disulfide stabilized Fv fragment (dsFv), a (dsFv)2, a Fv-Fc fusion, a scFv-Fc fusion, a scFv-Fv fusion, a diabody, a tribody, and a tetrabody. In some embodiments, the antibody moiety is a full-length antibody.

In some embodiments according to any of the anti-VISTA constructs described above, the antibody moiety has an Fc fragment that is selected from the group consisting of Fc fragments from IgG, IgA, IgD, IgE, IgM, and combinations and hybrids thereof. In some embodiments the Fc fragment is selected from the group consisting of Fc fragments from IgG1, IgG2, IgG3, IgG4, and combinations and hybrids thereof. In some embodiments, the Fc fragment has a reduced effector function as compared to the corresponding wildtype Fc fragment. In some embodiments, the Fc fragment has an extended half-life as compared to the corresponding wildtype Fc fragment.

In some embodiments according to any of the anti-VISTA constructs described above, the antibody moiety of the anti-VISTA construct activates the downstream signaling pathways of VISTA.

In some embodiments according to any of the anti-VISTA constructs described above, the anti-VISTA construct is an agonist antibody of VISTA. In some embodiments, the antibody moiety of the anti-VISTA construct activates or increases the downstream signaling pathways of VISTA by at least about 20%.

In some embodiments according to any of the anti-VISTA constructs described above, the VISTA is a human VISTA.

The present application in another aspect provides a pharmaceutical composition comprising an anti-VISTA construct described above, and a pharmaceutical acceptable carrier.

The present application in another aspect provides an isolated nucleic acid encoding an anti-VISTA construct described above.

The present application in another aspect provides a vector comprising an isolated nucleic acid sequence described above.

The present application in another aspect provides an isolated host cell comprising an isolated nucleic acid sequence or a vector described above.

The present application in another aspect provides an immunoconjugate comprising an anti-VISTA construct described above, linked to a therapeutic agent or a label.

The present application in another aspect provides a method of producing an anti-VISTA construct comprising: a) culturing an isolated host cell described above under conditions effective to express the anti-VISTA construct; and b) obtaining the expressed anti-VISTA construct from the host cell.

The present application in another aspect provides a method of treating a disease or condition in an individual, comprising administering to the individual an effective mount of an anti-VISTA construct or a pharmaceutical composition described above. In some embodiments, the disease of condition is associated with a dysregulated immune system. In some embodiments, the disease or condition is an auto-immune disease, inflammation, an infection, graft versus host disease (GvHD) or a condition associated with a transplant. In some embodiments, the auto-immune disease is selected from cutaneous lupus, rheumatoid arthritis, psoriasis, an autoimmune intestinal disorder, systemic lupus erythematosus (SLE), discoid lupus erythematosus (DLE). In some embodiments, the anti-VISTA construct is administered intravenously or subcutaneously into the individual. In some embodiments, the anti-VISTA construct is administered at a dose of about 0.001 mg/kg to about 100 mg/kg. In some embodiments, the individual is a human.

The present application in another aspect provides a kit comprising an anti-VISTA construct described above.

### BRIEF DESCRIPTION OF THE DRAWINGS

FIG. 1 shows anti-human VISTA antibody titers in serum of three immunized VISTA knockout mice by ELISA.

FIG. 2 shows anti-mouse VISTA antibody titers in serum of three immunized VISTA knockout mice by ELISA.

FIG. 3 depicts binding activities of various anti-VISTA antibodies against human, mouse and cynomolgus VISTA extracellular domains.

FIGS. 4A-4B depict binding activities of various anti-VISTA antibodies against Jurkat-hVISTA and Jurkat-mVISTA expressing cells using fluorescence activated cell sorting (FACS).

FIGS. 5A-5B depict activation of VISTA downstream pathway by 9F9 at various concentrations in Jurkat-NFKb-GFP/hVISTA-hCD3z expressing cells.

FIGS. 6A-6B depict activation of VISTA downstream pathway by 20E4 at various concentrations in Jurkat-NFKb-GFP/hVISTA-hCD3z expressing cells.

FIG. 7 compares various anti-VISTA antibodies at various concentrations in their capability of activating VISTA downstream pathway in Jurkat-NFKb-GFP/hVISTA-hCD3z expressing cells.

FIGS. 8A-8B depict activation of VISTA downstream pathway by 9F9 at various concentrations in the presence of an anti-CD3 antibody (OKT3) in Jurkat-NFKb-GFP/hVISTA-hCD3z expressing cells.

FIGS. 9A-9B depicts activation of VISTA downstream pathway by 20E4 at various concentrations in the presence of OKT3 in Jurkat-NFKb-GFP/hVISTA-hCD3z expressing cells.

FIG. 10 compares various anti-VISTA antibodies at various concentrations in their capability of activating VISTA downstream pathway in the presence of OKT3 in Jurkat-NFKb-GFP/hVISTA-hCD3z expressing cells.

FIGS. 11A-11B depict various recombinant mIgG1 anti-VISTA antibodies (9F9, 16A1, 17E7, 20E4, V4) specifically binds to Jurkat-hVISTA and Jurkat-mVISTA expressing cell lines using fluorescence activated cell sorting (FACS).

FIG. 12 depicts recombinant mIgG1 anti-VISTA (9F9) demonstrates activation in Jurkat-NFKb-GFP/hVISTA-hCD3z expressing cell lines using fluorescence activated cell sorting (FACS).

FIG. 13 depicts recombinant mIgG1 anti-VISTA (17E9) demonstrates activation in Jurkat-NFKb-GFP/hVISTA-hCD3z expressing cell lines using fluorescence activated cell sorting (FACS).

FIG. 14 depicts recombinant mIgG1 anti-VISTA (16A1) demonstrates activation in Jurkat-NFKb-GFP/hVISTA-hCD3z expressing cell lines using fluorescence activated cell sorting (FACS).

FIG. 15 depicts recombinant mIgG1 anti-VISTA (20E4) demonstrates activation in Jurkat-NFKb-GFP/hVISTA-hCD3z expressing cell lines using fluorescence activated cell sorting (FACS).

FIG. 16 depict epitope binning assay of anti-VISTA antibodies by Octet competition.

FIGS. 17A-17C depict human, cynomolgus and mouse VISTA antigen cross-binding activities of anti-VISTA mAbs by bio-layer interferometry (BLI) assay.

FIGS. 18A-18E depict binding activities of various anti-VISTA mAbs against human or cynomolgus VISTA.

FIG. 19 depicts inhibitory effects of anti-VISTA mAbs on T cell proliferation.

FIG. 20 depicts the summary of experimental protocol of mouse lupus treatment model.

FIG. 21A depicts lymph node enlargement in mice treated with mIgG, MHSA, 9F9 and 20E4 at 12, 14, 15 weeks of age, respectively. FIG. 21B depicts lymph nodes removed from neck area of one mouse in 20E4 group at 19 weeks.

FIG. 22 depicts serum levels of anti-nuclear immunoglobulins in mice upon treatment with mIgG, MHSA, 9F9 and 20E4.

FIG. 23 depicts serum levels of anti-dsDNA immunoglobulins in mice upon treatment with mIgG, MHSA, 9F9 and 20E4.

FIG. 24 depicts serum levels of IFNα in mice upon treatment with mIgG, MHSA, 9F9 and 20E4.

FIG. 25 depicts urine protein levels in mice of 12-week old upon treatment with mIgG, MHSA, 9F9 and 20E4.

FIG. 26 depicts urine protein levels in mice of 15-week old upon treatment with mIgG, MHSA, 9F9 and 20E4.

FIG. 27 depicts changes in skin lupus lesions of mice of 17-week old upon treatment with mIgG, MHSA, 9F9 and 20E4.

FIG. 28 depicts weight changes of mice injected with the isotype control versus those treated with the anti-VISTA antibodies 9F9 or 20E4.

FIG. 29 depicts levels of human CD45+cells in blood of mice injected with the isotype control versus those treated with the anti-VISTA antibodies 9F9 or 20E4.

FIG. 30 depicts the skin denudation in the mice injected with the isotype control versus those treated with the anti-VISTA antibodies 9F9 or 20E4.

### DETAILED DESCRIPTION OF THE APPLICATION

The present application provides novel anti-VISTA constructs that specifically bind to VISTA, methods of preparing the anti-VISTA constructs, methods of using the constructs (e.g., methods of treating a disease or condition). Exemplary anti-VISTA constructs include agonist antibodies that are capable of bind and activate VISTA.

### I. Definitions

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof.

A full-length antibody comprises two heavy chains and two light chains. The variable regions of the light and heavy chains are responsible for antigen binding. The variable domains of the heavy chain and light chain may be referred to as “V_{H}” and “V_{L}”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or A1-Lazikani (A1-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ϵ, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), IgA1 (α1 heavy chain), or lgA2 (α2 heavy chain). Chimeric Fc regions (such as IgG2/4 mixture) are also contemplated herein.

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelid single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above-cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein. In some embodiments, the CDR sequences provided herein are based on IMGT definition. For example, the CDR sequences may be determined by the VBASE2 tool (http://www.vbase2.org/vbase2.php, see also Retter I, Althaus HH, Münch R, Muller W: VBASE2, an integrative V gene database. Nucleic Acids Res. 2005 Jan 1; 33 (Database issue): D671-4, which is incorporated herein by reference in its entirety).

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Framework” or “FR” residues are those variable-domain residues other than the CDR residues as herein defined.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, See Jones et al., Nature 321:522-525 (1986); Riechmann et al., Nature 332:323-329 (1988); and Presta, Curr. Op. Struct. Biol. 2:593-596 (1992).

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the amino acid sequences TKLEIK and TALGIE share 50% homology. Generally, a comparison is made when two sequences are aligned to give maximum homology.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or C_{L}) domain of the light chain.

The “light chains” of antibodies (immunoglobulins) from any mammalian species can be assigned to one of two clearly distinct types, called kappa (“κ”) and lambda (“λ”), based on the amino acid sequences of their constant domains.

The “CH1 domain” (also referred to as “C1” of “H1” domain) usually extends from about amino acid 118 to about amino acid 215 (EU numbering system).

“Hinge region” is generally defined as a region in IgG corresponding to Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S-S bonds in the same positions.

The “CH2 domain” of a human IgG Fc region (also referred to as “C2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C2” domain) comprises the stretch of residues C-terminal to a CH2 domain in an Fc region (i.e. from about amino acid residue 341 to the C-terminal end of an antibody sequence, typically at amino acid residue 446 or 447 of an IgG).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. In some cases, the subsequent C-terminal glycine (residue 446 according to the EU numbering system) of the Fc region may also be removed. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Neonatal Fc receptors (FcRN) are encompassed herein. Other FcRs, including those to be identified in the future, are also encompassed by the term “FcR” herein.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody or fragment thereof “competes” for binding to a target antigen with a second antibody or fragment thereof when the first antibody or fragment thereof inhibits the target antigen binding of the second antibody of fragment thereof by at least about 50% (such as at least about any one of 55%, 60%, 65%, 70%, 75%, 80%, 85%, 90%, 95%, 98% or 99%) in the presence of an equimolar concentration of the first antibody or fragment thereof, or vice versa. A high throughput process for “binning” antibodies based upon their cross-competition is described in PCT Publication No. WO 03/48731.

As use herein, the terms “specifically binds,” “specifically recognizing,” and “is specific for” refer to measurable and reproducible interactions, such as binding between a target and an antibody or antibody moiety, which is determinative of the presence of the target in the presence of a heterogeneous population of molecules, including biological molecules. For example, an antibody or antibody moiety that specifically recognizes a target (which can be an epitope) is an antibody or antibody moiety that binds this target with greater affinity, avidity, more readily, and/or with greater duration than its bindings to other targets. In some embodiments, the extent of binding of an antibody to an unrelated target is less than about 10% of the binding of the antibody to the target as measured, e.g., by a radioimmunoassay (RIA). In some embodiments, an antibody that specifically binds a target has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, RIA-, ECL-, IRMA-, EIA-, BIACOR™-tests and peptide scans.

An “isolated” antibody (or construct) is one that has been identified, separated and/or recovered from a component of its production environment (e.g., natural or recombinant). Preferably, the isolated polypeptide is free of association with all other components from its production environment.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

The term “immunoconjugate” includes reference to a covalent linkage of a therapeutic agent or a detectable label to an antibody such as an antibody moiety described herein. The linkage can be direct or indirect through a linker (such as a peptide linker).

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. The methods of the application contemplate any one or more of these aspects of treatment.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to that of a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

As used herein, “delaying development of a disease” means to defer, hinder, slow, retard, stabilize, suppress and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease.

“Preventing” as used herein, includes providing prophylaxis with respect to the occurrence or recurrence of a disease in an individual that may be predisposed to the disease but has not yet been diagnosed with the disease.

The terms “subject,” “individual,” and “patient” are used interchangeably herein to refer to a mammal, including, but not limited to, human, bovine, horse, feline, canine, rodent, or primate. In some embodiments, the individual is a human.

An “effective amount” of an agent refers to an amount effective, at dosages and for periods of time necessary, to achieve the desired therapeutic or prophylactic result. The specific dose may vary depending on one or more of: the particular agent chosen, the dosing regimen to be followed, whether it is administered in combination with other compounds, timing of administration, the tissue to be imaged, and the physical delivery system in which it is carried.

The terms “pharmaceutical formulation” and “pharmaceutical composition” refer to a preparation which is in such form as to permit the biological activity of the active ingredient(s) to be effective, and which contains no additional components which are unacceptably toxic to an individual to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to an individual. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about 60 minutes, such as no more than about any of 30, 15, 10, 5, or 1 minutes.

The term “sequentially” is used herein to refer to administration of two or more therapeutic agents where the administration of one or more agent(s) continues after discontinuing the administration of one or more other agent(s). For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month, or longer.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder, or a probe for specifically detecting a biomarker described herein. In certain embodiments, the manufacture or kit is promoted, distributed, or sold as a unit for performing the methods described herein.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat a disease of type X means the method is used to treat the disease of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Anti-VISTA Constructs

The present application provides anti-VISTA constructs comprising an anti-VISTA antibody moiety that specifically binds to VISTA as described herein. VISTA

V domain Ig suppressor of T cell activation (VISTA) (also called PD-1H, Gi24, Dies-1, or DD 1α) is a more recently identified cell surface coinhibitory molecule of the CD28/B7 gene family. It has been reported that VISTA can function as an inhibitory ligand on antigen-presenting cells and regulate T cell responses, and ablation of VISTA by either genetic knockout or antagonist antibody can boost T cell immune responses against tumors in mouse models. VISTA may also play critical roles in the regulation of inflammation and autoimmune diseases, as shown in mouse models of graft-versus-host disease (GVHD), acute hepatitis, encephalitis, lupus, asthma, and psoriasis. VISTA can also function as a coinhibitory receptor on T cells. VISTA agonistic mAb dramatically regulates antigen-specific CD4 T cell responses and protects mice from GVHD, acute hepatitis, and asthma. See Files et al., J. Immunol. 187, 1537-1541 (2011); Files et al., J. Clin. Invest. 124, 1966-1975 (2014); and Liu et al., Cell. Mol. Immunol. 15, 838-845 (2018). Up-regulated VISTA in prostate cancer patients was also shown to associate with resistance to ipilimumab (CTLA-4 mAb). Furthermore, it has been shown that targeting VISTA may synergize with other nonredundant pathways, like PD-1 blockade, to achieve optimal tumor clearance efficacy in experimental mouse models. See Liu et al., Proc. Natl. Acad. Sci. U.S.A. 112, 6682-6687 (2015). Therefore, VISTA may be an important molecule in the regulation of immune responses and a potential target for immunotherapy.

VISTA gene is located on 10822.1. It is conserved in chimpanzee, cow, mouse, rat, chicken, zebrafish, and frog. Human VISTA sequence can be found with NCBI Reference number NM_022153. Human VISTA protein has 311 amino acids (NCBI Reference number: NP_071436.1, SEQ ID NO: 59.)

### Anti-VISTA Antibody Moieties

In some embodiments, the anti-VISTA construct comprises an antibody moiety comprising a heavy chain variable region (V_{H}) and a light chain variable region (V_{L}), wherein the antibody moiety competes for a binding epitope of VISTA with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6.

In some embodiments, the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 7; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 8.

In some embodiments, the V_{H} comprises an amino acid sequence of SEQ ID NO: 7, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 8, or a variant comprising an amino acid sequence having at least about 80% (such as at least about any one of 80%, 85%, 90%, 95%, 96%, 97%, 98%, or 99%) sequence identity.

In some embodiments, the anti-VISTA construct comprises an antibody moiety comprising a heavy chain variable region (V_{H}) and a light chain variable region (V_{L}), wherein the antibody moiety competes for a binding epitope of VISTA with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 9, the HC-CDR2 comprising the amino acid sequence of SEQ ID NO: 10, and the HC-CDR3 comprising the amino acid sequence of SEQ ID NO: 11, and the V_{L-2} comprises the LC-CDR1 comprising the amino acid sequence of SEQ ID NO: 12, the LC-CDR2 comprising the amino acid sequence of SEQ ID NO: 13, and the LC-CDR3 comprising the amino acid sequence of SEQ ID NO: 14.

In some embodiments, the V_{H} comprises i) the HC-CDR1 comprising the amino acid sequence of SEQ ID NO: 9, ii) the HC-CDR2 comprising the amino acid sequence of SEQ ID NO: 10, and iii) the HC-CDR3 comprising the amino acid sequence of SEQ ID NO: 11, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 12, ii) the LC-CDR2 comprising the amino acid sequence of SEQ ID NO: 13, and iii) the LC-CDR3 comprising the amino acid sequence of SEQ ID NO: 14, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 9, ii) the HC-CDR2 comprising the amino acid sequence of SEQ ID NO: 10, and iii) the HC-CDR3 comprising the amino acid sequence of SEQ ID NO: 11, and the V_{L} comprises i) the LC-CDR1 comprising the amino acid sequence of SEQ ID NO: 12, ii) the LC-CDR2 comprising the amino acid sequence of SEQ ID NO: 13, and iii) the LC-CDR3 comprising the amino acid sequence of SEQ ID NO: 14.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 15; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 16.

In some embodiments, the V_{H} comprises an amino acid sequence of SEQ ID NO: 15, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 16, or a variant comprising an amino acid sequence having at least about 80% (such as at least about any one of 80%, 85%, 90%, 95%, 96%, 97%, 98%, or 99%) sequence identity.

In some embodiments, the anti-VISTA construct comprises an antibody moiety comprising a heavy chain variable region (V_{H}) and a light chain variable region (V_{L}), wherein the antibody moiety competes for a binding epitope of VISTA with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 17, the HC-CDR2 comprising the amino acid sequence of SEQ ID NO: 18, and the HC-CDR3 comprising the amino acid sequence of SEQ ID NO: 19, and the V_{L-2} comprises the LC-CDR1 comprising the amino acid sequence of SEQ ID NO: 20, the LC-CDR2 comprising the amino acid sequence of SEQ ID NO: 21, and the LC-CDR3 comprising the amino acid sequence of SEQ ID NO: 22.

In some embodiments, the V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, and the V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 23; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 24.

In some embodiments, the V_{H} comprises an amino acid sequence of SEQ ID NO: 23, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 24, or a variant comprising an amino acid sequence having at least about 80% (such as at least about any one of 80%, 85%, 90%, 95%, 96%, 97%, 98%, or 99%) sequence identity.

In some embodiments, the anti-VISTA construct comprises an antibody moiety comprising a heavy chain variable region (V_{H}) and a light chain variable region (V_{L}), wherein the antibody moiety competes for a binding epitope of VISTA with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 25, the HC-CDR2 comprising the amino acid sequence of SEQ ID NO: 26, and the HC-CDR3 comprising the amino acid sequence of SEQ ID NO: 27, and the V_{L-2} comprises the LC-CDR1 comprising the amino acid sequence of SEQ ID NO: 28, the LC-CDR2 comprising the amino acid sequence of SEQ ID NO: 29, and the LC-CDR3 comprising the amino acid sequence of SEQ ID NO: 30.

In some embodiments, the V_{H} comprises i) the HC-CDR1 comprising the amino acid sequence of SEQ ID NO: 25, ii) the HC-CDR2 comprising the amino acid sequence of SEQ ID NO: 26, and iii) the HC-CDR3 comprising the amino acid sequence of SEQ ID NO: 27, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 28, ii) the LC-CDR2 comprising the amino acid sequence of SEQ ID NO: 29, and iii) the LC-CDR3 comprising the amino acid sequence of SEQ ID NO: 30, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 25, ii) the HC-CDR2 comprising the amino acid sequence of SEQ ID NO: 26, and iii) the HC-CDR3 comprising the amino acid sequence of SEQ ID NO: 27, and the V_{L} comprises i) the LC-CDR1 comprising the amino acid sequence of SEQ ID NO: 28, ii) the LC-CDR2 comprising the amino acid sequence of SEQ ID NO: 29, and iii) the LC-CDR3 comprising the amino acid sequence of SEQ ID NO: 30.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 31; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 32.

In some embodiments, the V_{H} comprises an amino acid sequence of SEQ ID NO: 31, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 32, or a variant comprising an amino acid sequence having at least about 80% (such as at least about any one of 80%, 85%, 90%, 95%, 96%, 97%, 98%, or 99%) sequence identity.

In some embodiments, the anti-VISTA construct comprises an antibody moiety comprising a heavy chain variable region (V_{H}) and a light chain variable region (V_{L}), wherein the antibody moiety competes for a binding epitope of VISTA with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L-2} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38.

In some embodiments, the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38.

In some embodiments, the antibody moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 39; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 40.

In some embodiments, the V_{H} comprises an amino acid sequence of SEQ ID NO: 39, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 40, or a variant comprising an amino acid sequence having at least about 80% (such as at least about any one of 80%, 85%, 90%, 95%, 96%, 97%, 98%, or 99%) sequence identity.

In some embodiments, the V_{H} comprises i) the HC-CDR1 comprising the amino acid sequence of SEQ ID NO: 41, ii) the HC-CDR2 comprising the amino acid sequence of SEQ ID NO: 42, and iii) the HC-CDR3 comprising the amino acid sequence of SEQ ID NO: 11, and the V_{L} comprises i) the LC-CDR1 comprising the amino acid sequence of SEQ ID NO: 46, ii) the LC-CDR2 comprising the amino acid sequence of SEQ ID NO: 13, and iii) the LC-CDR3 comprising the amino acid sequence of SEQ ID NO: 47.

In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 41, ii) the HC-CDR2 comprising the amino acid sequence of SEQ ID NO: 42, and iii) the HC-CDR3 comprising the amino acid sequence of SEQ ID NO: 11, and the V_{L} comprises i) the LC-CDR1 comprising the amino acid sequence of SEQ ID NO: 46, ii) the LC-CDR2 comprising the amino acid sequence of SEQ ID NO: 13, and iii) the LC-CDR3 comprising the amino acid sequence of SEQ ID NO: 47.

In some embodiments, the V_{H} comprises i) the HC-CDR1 comprising the amino acid sequence of SEQ ID NO: 43, ii) the HC-CDR2 comprising the amino acid sequence of SEQ ID NO: 44, and iii) the HC-CDR3 comprising the amino acid sequence of SEQ ID NO: 45, and the V_{L} comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 56.

In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 43, ii) the HC-CDR2 comprising the amino acid sequence of SEQ ID NO: 44, and iii) the HC-CDR3 comprising the amino acid sequence of SEQ ID NO: 45, and the V_{L} comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 56.

In some embodiments, the V_{H} comprises i) the HC-CDR1 comprising the amino acid sequence of SEQ ID NO: 43, ii) the HC-CDR2 comprising the amino acid sequence of SEQ ID NO: 52, and iii) the HC-CDR3 comprising the amino acid sequence of SEQ ID NO: 45, and the V_{L} comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 56.

In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 43, ii) the HC-CDR2 comprising the amino acid sequence of SEQ ID NO: 52, and iii) the HC-CDR3 comprising the amino acid sequence of SEQ ID NO: 45, and the V_{L} comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 56.

In some embodiments, the V_{H} comprises i) the HC-CDR1 comprising the amino acid sequence of SEQ ID NO: 43, ii) the HC-CDR2 comprising the amino acid sequence of SEQ ID NO: 52, and iii) the HC-CDR3 comprising the amino acid sequence of SEQ ID NO: 45, and the V_{L} comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 57.

In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 43, ii) the HC-CDR2 comprising the amino acid sequence of SEQ ID NO: 52, and iii) the HC-CDR3 comprising the amino acid sequence of SEQ ID NO: 45, and the V_{L} comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 57.

In some embodiments, the V_{H} comprises i) the HC-CDR1 comprising the amino acid sequence of SEQ ID NO: 43, ii) the HC-CDR2 comprising the amino acid sequence of SEQ ID NO: 44, and iii) the HC-CDR3 comprising the amino acid sequence of SEQ ID NO: 45, and the V_{L} comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 57.

In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 43, ii) the HC-CDR2 comprising the amino acid sequence of SEQ ID NO: 44, and iii) the HC-CDR3 comprising the amino acid sequence of SEQ ID NO: 45, and the V_{L} comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 57.

In some embodiments, the V_{H} comprises i) the HC-CDR1 comprising the amino acid sequence of SEQ ID NO: 43, ii) the HC-CDR2 comprising the amino acid sequence of SEQ ID NO: 58, and iii) the HC-CDR3 comprising the amino acid sequence of SEQ ID NO: 45, and the V_{L} comprises i) the LC-CDR1 comprising the amino acid sequence of SEQ ID NO: 48, ii) the LC-CDR2 comprising the amino acid sequence of SEQ ID NO: 49, and iii) the LC-CDR3 comprising the amino acid sequence of SEQ ID NO: 53.

In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 43, ii) the HC-CDR2 comprising the amino acid sequence of SEQ ID NO: 58, and iii) the HC-CDR3 comprising the amino acid sequence of SEQ ID NO: 45, and the V_{L} comprises i) the LC-CDR1 comprising the amino acid sequence of SEQ ID NO: 48, ii) the LC-CDR2 comprising the amino acid sequence of SEQ ID NO: 49, and iii) the LC-CDR3 comprising the amino acid sequence of SEQ ID NO: 53.

In some embodiments, the construct comprises or is an antibody or antigen-binding fragment thereof selected from the group consisting of a full-length antibody, a bispecific antibody, a single-chain Fv (scFv) fragment, a Fab fragment, a Fab' fragment, a F(ab')2, an Fv fragment, a disulfide stabilized Fv fragment (dsFv), a (dsFv)2, a V_{H} H, a Fv-Fc fusion, a scFv-Fc fusion, a scFv-Fv fusion, a diabody, a tribody, and a tetrabody.

In some embodiments, the anti-VISTA antibody moiety is a full-length antibody.

In some embodiments, the anti-VISTA antibody moiety is an scFv.

In some embodiments, the anti-VISTA antibody moiety described above comprises an Fc fragment of an immunoglobulin selected from the group consisting of IgG, IgA, IgD, IgE, IgM, and combinations and hybrids thereof. In some embodiments, the anti-VISTA antibody moiety or the full-length antibody described above comprises an Fc fragment of an immunoglobulin selected from the group consisting of IgG1, IgG2, IgG3, IgG4, and combinations and hybrids thereof. In some embodiments, the Fc fragment has a reduced effector function as compared to the corresponding wildtype Fc fragment. In some embodiments, the Fc fragment has an enhanced effector function as compared to the corresponding wildtype Fc fragment.

In some embodiments, the antibody moiety comprises a humanized antibody of any of the antibody moiety described herein.

In some embodiments, the anti-VISTA antibody moiety binds to both human VISTA and cynomolgus VISTA. In some embodiments, the anti-VISTA antibody moiety binds to both human VISTA and mouse VISTA. In some embodiments, the anti-VISTA antibody moiety binds to human VISTA, cynomolgus VISTA, and mouse VISTA. In some embodiments, the anti-VISTA antibody moiety does not bind to cynomolgus VISTA and/or mouse VISTA.

In some embodiments, the antibody moiety of the anti-VISTA construct activates the downstream signaling pathways of VISTA. In some embodiments, the anti-VISTA construct is an agonist antibody of VISTA.

In some embodiments, the antibody moiety of the anti-VISTA construct activates or increases the downstream signaling pathways of VISTA by at least about 5%, 10%, 15%, 20%, 25%, 30%, 35%, 40%, 45%, 50%, 55%, 60%, 65%, or 70% as compared to a reference construct (e.g., a corresponding construct that does not activate VISTA, e.g., a corresponding construct that comprises a reference agonist anti-VISTA antibody such as 1E8).

In some embodiments, the anti-VISTA construct comprises or is an anti-VISTA fusion protein. In some embodiments, the anti-VISTA construct comprises an anti-VISTA antibody moiety (e.g., an anti-VISTA scFv) and a second moiety. In some embodiments, the second moiety comprises a half-life extending moiety. In some embodiments, the half-life extending moiety is an albumin binding moiety (e.g., an albumin binding antibody moiety). In some embodiments, the anti-VISTA antibody moiety and the half-life extending moiety is linked via a linker (such as a peptide linker, such as a GS linker).

In some embodiments, the anti-VISTA construct comprises or is an anti-VISTA immunoconjugate comprising an anti-VISTA antibody moiety (such as any of the VISTA antibody moieties described herein) and a second agent. In some embodiments, the second agent is a therapeutic agent. In some embodiments, the second agent is a label.

In some embodiments, the VISTA is a human VISTA.

### a) Antibody Affinity

Binding specificity of the antibody moieties can be determined experimentally by methods known in the art. Such methods comprise, but are not limited to Western blots, ELISA-, RIA-, ECL-, IRMA-, EIA-, BIACORE™ -tests and peptide scans.

In some embodiments, the K_{D} of the binding between the antibody moiety and VISTA is about 10⁻⁷ M to about 10⁻¹² M, about 10⁻⁷ M to about 10 -8 M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁻⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M. In some embodiments, the K_{D} of the binding between the antibody moiety and VISTA is stronger than about any one of 10⁻⁷ M, 10⁻⁸ M, 10⁻⁹ M, 10⁻¹⁰ M, 10⁻¹¹ M, or 10⁻¹² M. In some embodiments, the VISTA is a human VISTA.

In some embodiments, the Kₒₙ of the binding between the antibody moiety and VISTA is about 10³ M⁻¹s⁻¹ to about 10⁸ M⁻¹s⁻¹, about 10³ M⁻¹s⁻¹ to about 10⁴ M⁻¹s⁻¹, about 10⁴ M⁻¹s⁻¹ to about 10⁵ M⁻¹s⁻¹, about 10⁵ M⁻¹s⁻¹ to about 10⁶ M⁻¹s⁻¹, about 10⁶ M⁻¹s⁻¹ to about 10⁻⁷ M⁻¹s⁻¹, or about 10⁷ M⁻¹s⁻¹ to about 10⁸ M⁻¹s⁻¹. In some embodiments, the Kₒₙ of the binding between the antibody moiety and VISTA is about 10³ M⁻¹s⁻¹ to about 10⁵ M⁻¹s⁻¹, about 10⁴ M⁻¹s⁻¹ to about 10⁶ M⁻¹s⁻¹, about 10⁵ M⁻¹s⁻¹ to about 10⁷ M⁻¹s⁻¹, about 10⁶ M⁻¹s⁻¹ to about 10⁸ M⁻1s⁻¹, about 10⁴ M⁻¹s⁻¹ to about 10⁷ M⁻¹s⁻¹, or about 10⁵ M⁻¹s⁻¹ to about 10⁸ M⁻¹s⁻¹. In some embodiments, the Kₒₙ of the binding between the antibody moiety and VISTA is no more than about any one of 10³ M⁻¹s⁻¹, 10⁴ M⁻¹M⁻¹s¹, 10⁵ M^{31 1}s⁻¹, 10⁶ M⁻¹s⁻¹, 10⁷ M⁻¹s⁻¹ or 10⁸ M⁻¹s⁻¹. In some embodiments, VISTA is human VISTA.

In some embodiments, the K_{off} of the binding between the antibody moiety and VISTA is about 1 s⁻¹ to about 10⁻⁶ s⁻¹, about 1 s⁻¹ to about 10⁻² s⁻¹, about 10⁻² s⁻¹ to about 10⁻³ s⁻¹, about 10⁻³ s⁻¹ to about 10⁻⁴ s⁻¹, about 10⁻⁴ s⁻¹ to about 10⁻⁵ s⁻¹, about 10⁻⁵ s⁻¹ to about 10⁻⁶ s⁻¹, about 1 s⁻¹ to about 10⁻⁵ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, about 10⁻² s⁻¹ to about 10⁻⁵ s⁻¹, or about 10⁻³ s⁻¹ to about 10⁻⁵ s⁻¹. In some embodiments, the K_{off} of the binding between the antibody moiety and VISTA is at least about any one of 1 s⁻¹, 10⁻² s⁻¹, 10⁻³ s⁻¹, 10⁻⁴ s⁻¹, 10⁻⁵ s⁻¹ or 10⁻⁶ s⁻¹. In some embodiments, VISTA is human VISTA.

In some embodiments, the binding affinity of the anti-VISTA antibody moiety or anti-VISTA construct are higher (for example, has a smaller K_{D} value) than an existing anti-VISTA antibody (e.g., anti-human VISTA antibody, e.g., 1E8).

### b) Chimeric or Humanized Antibodies

In some embodiments, the anti-VISTA antibody moiety is a chimeric antibody. In some embodiments, a chimeric antibody comprises a non-human variable region (e.g., a variable region derived from mouse) and a human constant region. In some embodiments, a chimeric antibody is a “class switched” antibody in which the class or subclass has been changed from that of the parent antibody. Chimeric antibodies include antigen-binding fragments thereof.

In some embodiments, the anti-VISTA antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5, 821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

It is understood that the humanization of mouse derived antibodies is a common and routinely used art. It is therefore understood that a humanized format of any and all of the anti-VISTA antibodies disclosed in Sequence Table can be used in a preclinical or clinical setting. In cases where a humanized format of any of the referenced anti-VISTA antibodies or their antigen-binding regions thereof is used in such a preclinical or clinical setting, the then humanized format is expected to bear the same or similar biological activities and profiles as the original non-humanized format.

### c) Human Antibodies

In some embodiments, the anti-VISTA antibody moiety is a human antibody (known as human domain antibody, or human DAb). Human antibodies can be produced using various techniques known in the art. Human antibodies are described generally in van Dijk and van de Winkel, Curr. Opin. Pharmacol. 5: 368-74 (2001), Lonberg, Curr. Opin. Immunol. 20:450-459 (2008), and Chen, Mol. Immunol. 47(4):912-21 (2010). Transgenic mice or rats capable of producing fully human single-domain antibodies (or DAb) are known in the art. See, e.g., US20090307787A1, U.S. Pat. No. 8,754,287, US20150289489A1, US20100122358A1, and W02004049794.

Human antibodies (e.g., human DAbs) may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VelociMouse® technology). Human variable regions from intact antibodies generated by such animals may be further modified, e.g., by combining with a different human constant region.

Human antibodies (e.g., human DAbs) can also be made by hybridoma-based methods. Human myeloma and mouse-human heteromyeloma cell lines for the production of human monoclonal antibodies have been described (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies (e.g., human DAbs) may also be generated by isolating Fv clone variable domain sequences selected from human-derived phage display libraries. Such variable domain sequences may then be combined with a desired human constant domain. Techniques for selecting human antibodies from antibody libraries are described below.

### d) Library-derived Antibodies

The anti-VISTA antibody moieties described herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004). Methods for constructing single-domain antibody libraries have been described, for example, See U.S. Pat. NO. 7371849.

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### e) Substitution, insertion, deletion and variants

In some embodiments, antibody variants having one or more amino acid substitutions are provided. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Amino acid substitutions

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### f) Glycosylation Variants

In some embodiments, the anti-VISTA antibody moiety is altered to increase or decrease the extent to which the construct is glycosylated. Addition or deletion of glycosylation sites to an antibody may be conveniently accomplished by altering the amino acid sequence such that one or more glycosylation sites is created or removed.

Where the antibody moiety comprises an Fc region, the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antibody moiety may be made in order to create antibody variants with certain improved properties.

In some embodiments, the anti-VISTA antibody moiety has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In some embodiments, the anti-VISTA antibody moiety has bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); US Patent No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### g) Fc Region Variants

In some embodiments, the anti-VISTA antibody moiety comprises an Fc fragment.

The term “Fc region,” “Fc domain,” “Fc fragment” or “Fc” refers to a C-terminal non-antigen binding region of an immunoglobulin heavy chain that contains at least a portion of the constant region. The term includes native Fc regions and variant Fc regions. In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, MD, 1991.

In some embodiments, the Fc fragment is from an immunoglobulin selected from the group consisting of IgG, IgA, IgD, IgE, IgM, and combinations and hybrids thereof. In some embodiments, the Fc fragment is from an immunoglobulin selected from the group consisting of IgG1, IgG2, IgG3, IgG4, and combinations and hybrids thereof.

In some embodiments, the Fc fragment has a reduced effector function as compared to corresponding wildtype Fc fragment (such as at least about 30%, 40%, 50%, 60%, 70%, 80%, 85%, 90%, or 95% reduced effector function as measured by the level of antibody-dependent cellular cytotoxicity (ADCC)).

In some embodiments, the Fc fragment is an IgG1 Fc fragment. In some embodiments, the IgG1 Fc fragment comprises a L234A mutation and/or a L235A mutation. In some embodiments the IgG1 Fc fragment comprises an L235A mutation and/or a G237A mutation. In some embodiments, the Fc fragment is an IgG2 or IgG4 Fc fragment. In some embodiments, the Fc fragment is an IgG4 Fc fragment comprising a S228P, F234A, and/or a L235A mutation. In some embodiments, the Fc fragment comprises a N297A mutation. In some embodiments, the Fc fragment comprises a N297G mutation.

In some embodiments, one or more amino acid modifications may be introduced into the Fc region of the antibody moiety, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the Fc fragment possesses some but not all effector functions, which make it a desirable candidate for applications in which the half-life of the antibody moiety in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (See Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). In some embodiments, the Fc fragment comprises a N297A mutation. In some embodiments, the Fc fragment comprises a N297G mutation.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, the Fc fragment is an IgG1 Fc fragment. In some embodiments, the IgG1 Fc fragment comprises a L234A mutation and/or a L235A mutation. In some embodiments, the IgG1 Fc fragment comprise an L235A mutation and/or a G237A mutation. In some embodiments, the Fc fragment is an IgG2 or IgG4 Fc fragment. In some embodiments, the Fc fragment is an IgG4 Fc fragment comprising a S228P, F234A, and/or a L235A mutation.

In some embodiments, the antibody moiety comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some embodiments, the Fc fragment has one or more mutations at Thr250, Met252, Ser254, The256, Thr307. Glu 380, Met428, His433, and/or Asn 434.

In some embodiments, the antibody moiety variant comprising a variant Fc region comprising one or more amino acid substitutions which alters half-life and/or changes binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which alters binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### h) Cysteine Engineered Aantibody Variants

In some embodiments, it may be desirable to create cysteine engineered antibody moieties, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: A118 (EU numbering) of the heavy chain; and S400 (EU numbering) of the heavy chain Fc region. Cysteine engineered antibody moieties may be generated as described, e.g., in U.S. Pat. No. 7,521,541.

### i) Antibody Derivatives

In some embodiments, the antibody moiety described herein may be further modified to comprise additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in diagnosis under defined conditions, etc.

In some embodiments, the antibody moiety may be further modified to comprise one or more biologically active protein, polypeptides or fragments thereof. “Bioactive” or “biologically active”, as used herein interchangeably, means showing biological activity in the body to carry out a specific function. For example, it may mean the combination with a particular biomolecule such as protein, DNA, etc., and then promotion or inhibition of the activity of such biomolecule. In some embodiments, the bioactive protein or fragments thereof include proteins and polypeptides that are administered to patients as the active drug substance for prevention of or treatment of a disease or condition, as well as proteins and polypeptides that are used for diagnostic purposes, such as enzymes used in diagnostic tests or in vitro assays, as well as proteins and polypeptides that are administered to a patient to prevent a disease such as a vaccine.

### III. Methods of preparation

In some embodiments, there is provided a method of preparing an anti-VISTA construct or antibody moiety that specifically binds to VISTA and a composition such as polynucleotide, nucleic acid construct, vector, host cell, or culture medium that is produced during the preparation of the anti-VISTA construct or antibody moiety. The anti-VISTA construct or antibody moiety or composition described herein may be prepared by a number of processes as generally described below and more specifically in the Examples.

### Antibody Expression and Production

The antibodies described herein can be prepared using any known methods in the art, including those described below and in the Examples.

### Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or a llama, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986). Also See Example 1 for immunization in Camels.

The immunizing agent will typically include the antigenic protein or a fusion variant thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. A cell sorter may also be used. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, HEK cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Pliickthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Nucleic Acid Molecules Encoding Antibody Moieties

In some embodiments, there is provided a polynucleotide encoding any one of the anti-VISTA constructs or antibody moieties described herein. In some embodiments, there is provided a polynucleotide prepared using any one of the methods as described herein. In some embodiments, a nucleic acid molecule comprises a polynucleotide that encodes a heavy chain or a light chain of an antibody moiety (e.g., anti-VISTA antibody moiety). In some embodiments, a nucleic acid molecule comprises both a polynucleotide that encodes a heavy chain and a polynucleotide that encodes a light chain, of an antibody moiety (e.g., anti-VISTA antibody moiety). In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody moiety (e.g., anti-VISTA antibody moiety) comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

In some embodiments, the polynucleotide is a DNA. In some embodiments, the polynucleotide is an RNA. In some embodiments, the RNA is an mRNA.

Nucleic acid molecules may be constructed using recombinant DNA techniques conventional in the art. In some embodiments, a nucleic acid molecule is an expression vector that is suitable for expression in a selected host cell. Nucleic acid construct

In some embodiments, there is provided a nucleic acid construct comprising any one of the polynucleotides described herein. In some embodiments, there is provided a nucleic acid construct prepared using any method described herein.

In some embodiments, the nucleic acid construct further comprises a promoter operably linked to the polynucleotide. In some embodiments, the polynucleotide corresponds to a gene, wherein the promoter is a wild-type promoter for the gene. Vectors

In some embodiments, there is provided a vector comprising any polynucleotides that encode the heavy chains and/or light chains of any one of the antibody moieties described herein (e.g., anti-VISTA antibody moieties) or nucleic acid construct described herein. In some embodiments, there is provided a vector prepared using any method described herein. Vectors comprising polynucleotides that encode any of anti-VISTA constructs such as antibodies, scFvs, fusion proteins or other forms of constructs described herein (e.g., anti-VISTA scFv) are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004).

### Host Cells

In some embodiments, there is provided a host cell comprising any polypeptide, nucleic acid construct and/or vector described herein. In some embodiments, there is provided a host cell prepared using any method described herein. In some embodiments, the host cell is capable of producing any of antibody moieties described herein under a fermentation condition.

In some embodiments, the antibody moieties described herein (e.g., anti-VISTA antibody moieties) may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, CHO-GS, DG44. Lec13 CHO cells, and FUT8 CHO cells; PER.C6® cells (Crucell); HEK cells, and NSO cells. In some embodiments, the antibody moieties described herein (e.g., anti-VISTA antibody moieties) may be expressed in yeast. See, e.g., U.S. Publication No. US 2006/0270045 A1. In some embodiments, a particular eukaryotic host cell is selected based on its ability to make desired post-translational modifications to the heavy chains and/or light chains of the antibody moiety. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

The present application also provides host cells comprising any of the polynucleotides or vectors described herein. In some embodiments, the invention provides a host cell comprising an anti-VISTA antibody. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

In some embodiments, the antibody moiety is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### Culture Medium

In some embodiments, there is provided a culture medium comprising any antibody moiety, polynucleotide, nucleic acid construct, vector, and/or host cell described herein. In some embodiments, there is provided a culture medium prepared using any method described herein.

In some embodiments, the medium comprises hypoxanthine, aminopterin, and/or thymidine (e.g., HAT medium). In some embodiments, the medium does not comprise serum. In some embodiments, the medium comprises serum. In some embodiments, the medium is a D-MEM or RPMI-1640 medium.

In some embodiments the culture medium is chemically defined. In some embodiments the culture medium is specifically derived for a specific cell line (e.g., CHO GS cells).

### Purification of Antibody Moieties

The anti-VISTA constructs may be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify an anti-VISTA construct comprising an Fc fragment. Hydrophobic interactive chromatography, for example, a butyl or phenyl column, may also suitable for purifying some polypeptides such as antibodies. Ion exchange chromatography (e.g. anion exchange chromatography and/or cation exchange chromatography) may also suitable for purifying some polypeptides such as antibodies. Mixed-mode chromatography (e.g. reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

### V. Methods of Treatments

Also provided here are methods of treating a disease or condition or modulating an immune response (e.g., inhibiting proliferation of a T cell) in an individual or modulating an immune response (e.g., inhibiting proliferation of a T cell) in an individual. The methods comprise administering the anti-VISTA construct described herein into individuals (e.g., mammals such as humans).

In some embodiments, there is provided a method of treating a disease or condition or modulating an immune response (e.g., inhibiting proliferation of a T cell) in an individual, comprising administering to the individual an effective amount of an anti-VISTA construct described herein. In some embodiments, the disease or condition is associated with a dysregulated immune system. In some embodiments, the disease or condition is an auto-immune disease, inflammation, an infection, graft versus host disease (GvHD) or a condition associated with a transplant. In some embodiments, the auto-immune disease is selected from cutaneous lupus, rheumatoid arthritis, psoriasis, an autoimmune intestinal disorder, systemic lupus erythematosus (SLE), discoid lupus erythematosus (DLE).

In some embodiments, there is provided a method of treating a disease or condition or modulating an immune response (e.g., inhibiting proliferation of a T cell) in an individual, comprising administering to the individual an effective mount of the anti-VISTA construct comprising an antibody moiety comprising a heavy chain variable region (V_{H}) and a light chain variable region (V_{L}), wherein the antibody moiety competes for a binding epitope of VISTA with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6. In some embodiments, the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs. In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6. In some embodiments, the V_{H} comprises an amino acid sequence of SEQ ID NO: 7, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 8, or a variant comprising an amino acid sequence having at least about 80% (such as at least about any one of 80%, 85%, 90%, 95%, 96%, 97%, 98%, or 99%) sequence identity. In some embodiments, the disease or condition is associated with a dysregulated immune system. In some embodiments, the disease or condition is an auto-immune disease, inflammation, an infection, graft versus host disease (GvHD) or a condition associated with a transplant. In some embodiments, the auto-immune disease is selected from cutaneous lupus, rheumatoid arthritis, psoriasis, an autoimmune intestinal disorder, systemic lupus erythematosus (SLE), discoid lupus erythematosus (DLE).

In some embodiments, there is provided a method of treating a disease or condition or modulating an immune response (e.g., inhibiting proliferation of a T cell) in an individual, comprising administering to the individual an effective mount of the anti-VISTA construct comprising an antibody moiety comprising a heavy chain variable region (V_{H}) and a light chain variable region (V_{L}), wherein the antibody moiety competes for a binding epitope of VISTA with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 9, the HC-CDR2 comprising the amino acid sequence of SEQ ID NO: 10, and the HC-CDR3 comprising the amino acid sequence of SEQ ID NO: 11, and the V_{L-2} comprises the LC-CDR1 comprising the amino acid sequence of SEQ ID NO: 12, the LC-CDR2 comprising the amino acid sequence of SEQ ID NO: 13, and the LC-CDR3 comprising the amino acid sequence of SEQ ID NO: 14. In some embodiments, the V_{H} comprises i) the HC-CDR1 comprising the amino acid sequence of SEQ ID NO: 9, ii) the HC-CDR2 comprising the amino acid sequence of SEQ ID NO: 10, and iii) the HC-CDR3 comprising the amino acid sequence of SEQ ID NO: 11, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 12, ii) the LC-CDR2 comprising the amino acid sequence of SEQ ID NO: 13, and iii) the LC-CDR3 comprising the amino acid sequence of SEQ ID NO: 14, or a variant thereof comprising up to 5, 4, 3, 2, or 1 amino acid substitutions in the LC-CDRs. In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 9, ii) the HC-CDR2 comprising the amino acid sequence of SEQ ID NO: 10, and iii) the HC-CDR3 comprising the amino acid sequence of SEQ ID NO: 11, and the V_{L} comprises i) the LC-CDR1 comprising the amino acid sequence of SEQ ID NO: 12, ii) the LC-CDR2 comprising the amino acid sequence of SEQ ID NO: 13, and iii) the LC-CDR3 comprising the amino acid sequence of SEQ ID NO: 14. In some embodiments, the V_{H} comprises an amino acid sequence of SEQ ID NO: 15, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 16, or a variant comprising an amino acid sequence having at least about 80% (such as at least about any one of 80%, 85%, 90%, 95%, 96%, 97%, 98%, or 99%) sequence identity. In some embodiments, the disease or condition is associated with a dysregulated immune system. In some embodiments, the disease or condition is an auto-immune disease, inflammation, an infection, graft versus host disease (GvHD) or a condition associated with a transplant. In some embodiments, the auto-immune disease is selected from cutaneous lupus, rheumatoid arthritis, psoriasis, an autoimmune intestinal disorder, systemic lupus erythematosus (SLE), discoid lupus erythematosus (DLE).

In some embodiments, there is provided a method of treating a disease or condition or modulating an immune response (e.g., inhibiting proliferation of a T cell) in an individual, comprising administering to the individual an effective mount of the anti-VISTA construct comprising an antibody moiety comprising a heavy chain variable region (V_{H}) and a light chain variable region (V_{L}), wherein the antibody moiety competes for a binding epitope of VISTA with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 17, the HC-CDR2 comprising the amino acid sequence of SEQ ID NO: 18, and the HC-CDR3 comprising the amino acid sequence of SEQ ID NO: 19, and the V_{L-2} comprises the LC-CDR1 comprising the amino acid sequence of SEQ ID NO: 20, the LC-CDR2 comprising the amino acid sequence of SEQ ID NO: 21, and the LC-CDR3 comprising the amino acid sequence of SEQ ID NO: 22. In some embodiments, the V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22, or a variant thereof comprising up to 5, 4, 3, 2, or 1 amino acid substitutions in the LC-CDRs. In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, and the V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22. In some embodiments, the V_{H} comprises an amino acid sequence of SEQ ID NO: 23, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 24, or a variant comprising an amino acid sequence having at least about 80% (such as at least about any one of 80%, 85%, 90%, 95%, 96%, 97%, 98%, or 99%) sequence identity. In some embodiments, the disease or condition is associated with a dysregulated immune system. In some embodiments, the disease or condition is an auto-immune disease, inflammation, an infection, graft versus host disease (GvHD) or a condition associated with a transplant. In some embodiments, the auto-immune disease is selected from cutaneous lupus, rheumatoid arthritis, psoriasis, an autoimmune intestinal disorder, systemic lupus erythematosus (SLE), discoid lupus erythematosus (DLE).

In some embodiments, there is provided a method of treating a disease or condition or modulating an immune response (e.g., inhibiting proliferation of a T cell) in an individual, comprising administering to the individual an effective mount of the anti-VISTA construct comprising an antibody moiety comprising a heavy chain variable region (V_{H}) and a light chain variable region (V_{L}), wherein the antibody moiety competes for a binding epitope of VISTA with an antibody or antibody fragment comprising a second heavy variable region (VH-2) and a second light chain variable region (V_{L-2}), wherein the V_{H-2} comprises the HC-CDR1 comprising the amino acid sequence of SEQ ID NO: 25, the HC-CDR2 comprising the amino acid sequence of SEQ ID NO: 26, and the HC-CDR3 comprising the amino acid sequence of SEQ ID NO: 27, and the V_{L-2} comprises the LC-CDR1 comprising the amino acid sequence of SEQ ID NO: 28, the LC-CDR2 comprising the amino acid sequence of SEQ ID NO: 29, and the LC-CDR3 comprising the amino acid sequence of SEQ ID NO: 30. In some embodiments, the V_{H} comprises i) the HC-CDR1 comprising the amino acid sequence of SEQ ID NO: 25, ii) the HC-CDR2 comprising the amino acid sequence of SEQ ID NO: 26, and iii) the HC-CDR3 comprising the amino acid sequence of SEQ ID NO: 27, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 28, ii) the LC-CDR2 comprising the amino acid sequence of SEQ ID NO: 29, and iii) the LC-CDR3 comprising the amino acid sequence of SEQ ID NO: 30, or a variant thereof comprising up to 5, 4, 3, 2, or 1 amino acid substitutions in the LC-CDRs. In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 25, ii) the HC-CDR2 comprising the amino acid sequence of SEQ ID NO: 26, and iii) the HC-CDR3 comprising the amino acid sequence of SEQ ID NO: 27, and the V_{L} comprises i) the LC-CDR1 comprising the amino acid sequence of SEQ ID NO: 28, ii) the LC-CDR2 comprising the amino acid sequence of SEQ ID NO: 29, and iii) the LC-CDR3 comprising the amino acid sequence of SEQ ID NO: 30. In some embodiments, the V_{H} comprises an amino acid sequence of SEQ ID NO: 31, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 32, or a variant comprising an amino acid sequence having at least about 80% (such as at least about any one of 80%, 85%, 90%, 95%, 96%, 97%, 98%, or 99%) sequence identity. In some embodiments, the disease or condition is associated with a dysregulated immune system. In some embodiments, the disease or condition is an auto-immune disease, inflammation, an infection, graft versus host disease (GvHD) or a condition associated with a transplant. In some embodiments, the auto-immune disease is selected from cutaneous lupus, rheumatoid arthritis, psoriasis, an autoimmune intestinal disorder, systemic lupus erythematosus (SLE), discoid lupus erythematosus (DLE).

In some embodiments, there is provided a method of treating a disease or condition or modulating an immune response (e.g., inhibiting proliferation of a T cell) in an individual, comprising administering to the individual an effective mount of the anti-VISTA construct comprising an antibody moiety comprising a heavy chain variable region (V_{H}) and a light chain variable region (V_{L}), wherein the antibody moiety competes for a binding epitope of VISTA with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L-2} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38. In some embodiments, the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising up to 5, 4, 3, 2, or 1 amino acid substitutions in the LC-CDRs. In some embodiments, the anti-VISTA antibody moiety is a humanized antibody derived from an anti-VISTA antibody comprising a heavy chain variable region (V_{H}) and a light chain variable region (V_{L}), wherein the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38. In some embodiments, the V_{H} comprises an amino acid sequence of SEQ ID NO: 39, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 40, or a variant comprising an amino acid sequence having at least about 80% (such as at least about any one of 80%, 85%, 90%, 95%, 96%, 97%, 98%, or 99%) sequence identity. In some embodiments, the disease or condition is associated with a dysregulated immune system. In some embodiments, the disease or condition is an auto-immune disease, inflammation, an infection, graft versus host disease (GvHD) or a condition associated with a transplant. In some embodiments, the auto-immune disease is selected from cutaneous lupus, rheumatoid arthritis, psoriasis, an autoimmune intestinal disorder, systemic lupus erythematosus (SLE), discoid lupus erythematosus (DLE).

In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, there is provided a method of treating a disease or condition or modulating an immune response (e.g., inhibiting proliferation of a T cell) in an individual, comprising administering to the individual an effective mount of the anti-VISTA construct comprising an antibody moiety comprising a heavy chain variable region (VH) and a light chain variable region (VL), wherein the VH comprises i) the HC-CDR1 comprising the amino acid sequence of SEQ ID NO: 41, ii) the HC-CDR2 comprising the amino acid sequence of SEQ ID NO: 42 or 51, and iii) the HC-CDR3 comprising the amino acid sequence of SEQ ID NO: 11; and the VL comprises i) the LC-CDR1 comprising the amino acid sequence of SEQ ID NO: 46, ii) the LC-CDR2 comprising the amino acid sequence of SEQ ID NO: 13, and iii) the LC-CDR3 comprising the amino acid sequence of SEQ ID NO: 47.

In some embodiments, there is provided a method of treating a disease or condition or modulating an immune response (e.g., inhibiting proliferation of a T cell) in an individual, comprising administering to the individual an effective amount of the anti-VISTA construct comprising an antibody moiety comprising a heavy chain variable region (VH) and a light chain variable region (VL), wherein the VH comprises i) the HC-CDR1 comprising the amino acid sequence of SEQ ID NO: 43, ii) the HC-CDR2 comprising the amino acid sequence of SEQ ID NO: 44 or 52, and iii) the HC-CDR3 comprising the amino acid sequence of SEQ ID NO: 45; and the VL comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 56 or 57. In some embodiments, the disease or condition is associated with a dysregulated immune system. In some embodiments, the disease or condition is an auto-immune disease, inflammation, an infection, graft versus host disease (GvHD) or a condition associated with a transplant. In some embodiments, the auto-immune disease is selected from cutaneous lupus, rheumatoid arthritis, psoriasis, an autoimmune intestinal disorder, systemic lupus erythematosus (SLE), discoid lupus erythematosus (DLE).

In some embodiments, there is provided a method of treating a disease or condition or modulating an immune response (e.g., inhibiting proliferation of a T cell) in an individual, comprising administering to the individual an effective mount of the anti-VISTA construct comprising an antibody moiety comprising a heavy chain variable region (VH) and a light chain variable region (VL), wherein the VH comprises i) the HC-CDR1 comprising the amino acid sequence of SEQ ID NO: 41 or 43, ii) the HC-CDR2 comprising the amino acid sequence of any of SEQ ID NO:58, and iii) the HC-CDR3 comprising the amino acid sequence of SEQ ID NO: 11 or 45; and the VL comprises i) the LC-CDR1 comprising the amino acid sequence of SEQ ID NO: 48, ii) the LC-CDR2 comprising the amino acid sequence of SEQ ID NO: 49, and iii) the LC-CDR3 comprising the amino acid sequence of SEQ ID NO: 50 or 53. In some embodiments, the disease or condition is associated with a dysregulated immune system. In some embodiments, the disease or condition is an auto-immune disease, inflammation, an infection, graft versus host disease (GvHD) or a condition associated with a transplant. In some embodiments, the auto-immune disease is selected from cutaneous lupus, rheumatoid arthritis, psoriasis, an autoimmune intestinal disorder, systemic lupus erythematosus (SLE), discoid lupus erythematosus (DLE).

In some embodiments, the anti-VISTA construct used in modulating an immune response in an individual or modulating an immune cell (e.g., a T cell) prevents the proliferation of T cells by at least about 10%, 20%, 30%, 40%, 50%, 60%, 70%, 80%, or 90% as compared to a corresponding construct that does not activate VISTA (e.g., an isotype control). In some embodiments, the anti-VISTA construct used in modulating an immune response in an individual or modulating an immune cell (e.g., a T cell) prevents the proliferation of T cells by at least about 5%, 10%, 15%, 20%, or 25% as compared to a corresponding construct that comprises a reference agonist anti-VISTA antibody (e.g., 1E8).

In some embodiments, there is provided a method of modulating a cell (e.g., an immune cell), comprising contacting the immune cell with an anti-VISTA construct (such as any of the anti-VISTA constructs described herein. In some embodiments, the cell is a T cell (such as CD4 and/or CD8 T cell). In some embodiments, the cell is a neutrophil. In some embodiments, the cell is a dendritic cell (e.g., plasmacytoid dendritic cell). In some embodiments, the cell is a macrophage. In some embodiments, the contacting occurs in vitro.

In some embodiments, there is provided a method of genome-editing a cell (e.g., an immune cell), comprising introducing the cell: a) a donor template comprising a nucleic acid sequence encoding any of the anti-VISTA constructs described herein, and b) a DNA nuclease or nucleotide sequence encoding the DNA nuclease (e.g., a CRISPR-associated protein (Cas)). In some embodiments, the method further comprises administering the genome-edited cell into an individual having a disease or condition described herein.

In some embodiments, the subject is a mammal (such as a human).

In some embodiments, the individual has an elevated serum level of anti-nuclear antibodies (e.g., at least about 20%, 40%, 60%, 80%, 100%, 150%, 200%, 300%, 400%, or 500% higher serum level anti-nuclear antibodies than that of a healthy individual). In some embodiments, the individual has an elevated serum level of anti-dsDNA antibodies (e.g., at least about 20%, 40%, 60%, 80%, 100%, 150%, 200%, 300%, 400%, or 500% higher serum level anti-dsDNA antibodies than that of a healthy individual). In some embodiments, the individual has an elevated serum level of IFNα (e.g., at least about 20%, 40%, 60%, 80%, 100%, 150%, 200%, 300%, 400%, or 500% higher serum level of IFNα than that of a healthy individual). In some embodiments, the individual has an elevated protein level in urine (e.g., at least about 20%, 40%, 60%, 80%, 100%, 150%, 200%, 300%, 400%, or 500% higher level of protein in urine than that of a healthy individual).

### Dosing and Method of Administering the anti-VISTA Construct

The dosing regimen of the anti-VISTA construct (such as the specific dosages and frequencies) used for treating a disease or disorder as described herein administered into the individual may vary with the particular anti-VISTA construct, the mode of administration, and the type of disease or condition being treated. In some embodiments, the effective amount of the anti-VISTA construct is an amount that is effective to alleviate at least one symptom of the disease or condition. In some embodiments, the effective amount of the anti-VISTA construct is an amount that is sufficient to prolong overall survival of the individual. In some embodiments, the effective amount of the anti-VISTA construct is an amount that is sufficient to produce clinical benefit of more than about any of 50%, 60%, 70%, 80%, or 90% among a population of individuals treated with the anti-VISTA construct.

In some embodiments, the effective amount of the anti-VISTA construct is an amount that slows or inhibits the progression of the disease or condition (for example, by at least about 5%, 10%, 15%, 20%, 30%, 40%, 50%) as compared to that of the individual not receiving the treatment. In some embodiments, the disease or condition is an autoimmune disease. In some embodiments, the disease or condition is an infection.

In some embodiments, the effective amount of the anti-VISTA construct reduces serum level of anti-nuclear antibodies by at least about 10%, 15%, 20%, 25%, 30%, 35%, 40%, 45%, 50%, 55%, 60%, 65%, or 70% compared to that of a reference individual (e.g., an individual having the same disease or condition but not treated with the anti-VISTA construct). In some embodiments, the effective amount of the anti-VISTA construct reduces serum level of anti-dsDNA antibodies by at least about 10%, 15%, 20%, 25%, 30%, 35%, 40%, 45%, 50%, 55%, 60%, 65%, or 70% compared to that of a reference individual (e.g., an individual having the same disease or condition but not treated with the anti-VISTA construct). In some embodiments, the effective amount of the anti-VISTA construct reduces serum level of IFNα by at least about 10%, 15%, 20%, 25%, 30%, 35%, 40%, 45%, 50%, 55%, 60%, 65%, or 70% compared to that of a reference individual (e.g., an individual having the same disease or condition but not treated with the anti-VISTA construct). In some embodiments, the effective amount of the anti-VISTA construct reduces protein levels in urine by at least about 10%, 15%, 20%, 25%, 30%, 35%, 40%, 45%, 50%, 55%, 60%, 70%, 80%, or 90% compared to that of a reference individual (e.g., an individual having the same disease or condition but not treated with the anti-VISTA construct).

In some embodiments, the effective amount of the anti-VISTA construct is an amount that reduces the side effects (auto-immune response) of a condition (e.g., transplantation) (for example, by at least about 5%, 10%, 15%, 20%, 30%, 40%, or 50%) as compared to that of the individual not receiving the treatment.

In some embodiments of any of the above aspects, the effective amount of an anti-VISTA construct is in the range of about 0.001 μg/kg to about 100 mg/kg of total body weight, for example, about 0.005 vg/kg to about 50 mg/kg, about 0.01 vg/kg to about 10 mg/kg, or about 0.01 vg/kg to about 1 mg/kg.

In some embodiments of any of the above aspects, the effective amount of an anti-VISTA construct for a human is a dose that is equivalent to 0.5 mg for a mouse.

In some embodiments of any of the above aspects, the anti-VISTA construct is administered weekly. In some embodiments of any of the above aspects, the anti-VISTA construct is administered bi-weekly. In some embodiments, the anti-VISTA construct is administered weekly for at least about 2, about 4, about 6, about 8, about 10, about 12, about 14, about 16, about 18, or about 20 weeks.

The anti-VISTA construct can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, the anti-VISTA construct is included in a pharmaceutical composition while administered into the individual.

In some embodiments, sustained continuous release formulation of the composition may be used. In some embodiments, the composition is administered intravenously. In some embodiments, the composition is administered intraperitoneally. In some embodiments, the composition is administered intravenously. In some embodiments, the composition is administered intraperitoneally. In some embodiments, the composition is administered intramuscularly. In some embodiments, the composition is administered subcutaneously. In some embodiments, the composition is administered intravenously. In some embodiments, the composition is administered orally.

### Combination Therapy

This application also provides methods of administering an anti-VISTA construct into an individual for treating a disease or condition, wherein the method further comprises administering a second agent or therapy. In some embodiments, the second agent or therapy is a standard or commonly used agent or therapy for treating the disease or condition.

In some embodiments, the anti-VISTA construct is administered simultaneously with the second agent or therapy. In some embodiments, the anti-VISTA construct is administered concurrently with the second agent or therapy. In some embodiments, the anti-VISTA construct is administered sequentially with the second agent or therapy. In some embodiments, the anti-VISTA construct is administered prior to the second agent or therapy. In some embodiments, the anti-VISTA construct is administered after the second agent or therapy. In some embodiments, the anti-VISTA construct is administered in the same unit dosage form as the second agent or therapy. In some embodiment, the anti-VISTA construct is administered in a different unit dosage form from the second agent or therapy. In some embodiments, the anti-VISTA construct is administered in the same unit dosage form as the second agent or therapy. In some embodiment, the anti-VISTA construct is administered in a different unit dosage form from the second agent or therapy.

### VI. Compositions, Kits and Articles of Manufacture

Also provided herein are compositions (such as formulations) comprising any one of the anti-VISTA construct or anti-VISTA antibody moiety described herein, nucleic acid encoding the antibody moieties, vector comprising the nucleic acid encoding the antibody moieties, or host cells comprising the nucleic acid or vector.

Suitable formulations of the anti-VISTA construct described herein can be obtained by mixing the anti-VISTA construct or anti-VISTA antibody moiety having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be imaged, diagnosed, or treated herein.

The formulations to be used for in vivo administration must be sterile. This is readily accomplished by, e.g., filtration through sterile filtration membranes.

Also provided are kits comprising any one of the anti-VISTA construct or anti-VISTA antibody moiety described herein. The kits may be useful for any of the methods of modulating cell composition or treatment described herein.

In some embodiments, there is provided a kit comprising an anti-VISTA construct specifically binding to VISTA.

In some embodiments, the kit further comprises a device capable of delivering the anti-VISTA construct into an individual. One type of device, for applications such as parenteral delivery, is a syringe that is used to inject the composition into the body of a subject. Inhalation devices may also be used for certain applications.

In some embodiments, the kit further comprises a therapeutic agent for treating a disease or condition, e.g., infectious disease, autoimmune disease, or transplantation.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present application thus also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle). The label or package insert indicates that the composition is used for imaging, diagnosing, or treating a particular condition in an individual. The label or package insert will further comprise instructions for administering the composition to the individual and for imaging the individual. The label may indicate directions for reconstitution and/or use. The container holding the composition may be a multi-use vial, which allows for repeat administrations (e.g. from 2-6 administrations) of the reconstituted formulation. Package insert refers to instructions customarily included in commercial packages of diagnostic products that contain information about the indications, usage, dosage, administration, contraindications and/or warnings concerning the use of such diagnostic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the compositions and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXEMPLARY EMBODIMENTS

Embodiment 1. An anti-VISTA construct comprising an antibody moiety comprising a heavy chain variable region (VH) and a light chain variable region (VL), wherein the antibody moiety competes for a binding epitope of VISTA with an antibody or antibody fragment comprising a second heavy chain variable region (VH-2) and a second light chain variable region (VL-2), wherein:

a) the VH-2 comprising the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the VL-2¬¬ comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6;

b) the VH-2 comprises the HC-CDR1 comprising the amino acid sequence of SEQ ID NO: 9, the HC-CDR2 comprising the amino acid sequence of SEQ ID NO: 10, and the HC-CDR3 comprising the amino acid sequence of SEQ ID NO: 11, and the VL-2 comprises the LC-CDR1 comprising the amino acid sequence of SEQ ID NO: 12, the LC-CDR2 comprising the amino acid sequence of SEQ ID NO: 13, and the LC-CDR3 comprising the amino acid sequence of SEQ ID NO: 14;

c) the VH-2 comprises the HC-CDR1 comprising the amino acid sequence of SEQ ID NO: 17, the HC-CDR2 comprising the amino acid sequence of SEQ ID NO: 18, and the HC-CDR3 comprising the amino acid sequence of SEQ ID NO: 19, and the VL-2 comprises the LC-CDR1 comprising the amino acid sequence of SEQ ID NO: 20, the LC-CDR2 comprising the amino acid sequence of SEQ ID NO: 21, and the LC-CDR3 comprising the amino acid sequence of SEQ ID NO: 22;

d) the VH-2 comprises the HC-CDR1 comprising the amino acid sequence of SEQ ID NO: 25, the HC-CDR2 comprising the amino acid sequence of SEQ ID NO: 26, and the HC-CDR3 comprising the amino acid sequence of SEQ ID NO: 27, and the VL-2 comprises the LC-CDR1 comprising the amino acid sequence of SEQ ID NO: 28, the LC-CDR2 comprising the amino acid sequence of SEQ ID NO: 29, and the LC-CDR3 comprising the amino acid sequence of SEQ ID NO: 30; or

e) the VH-2 comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the VL-2 comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38.

Embodiment 2. The anti-VISTA construct of embodiment 1, wherein:

a) the VH comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising 5, 4, 3, 2, or 1 amino acid substitutions in the HC-CDRs; and the VL comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising 5, 4, 3, 2, or 1 amino acid substitutions in the LC-CDRs;

b) the VH comprises i) the HC-CDR1 comprising the amino acid sequence of SEQ ID NO: 9, ii) the HC-CDR2 comprising the amino acid sequence of SEQ ID NO: 10, and iii) the HC-CDR3 comprising the amino acid sequence of SEQ ID NO: 11, or a variant thereof comprising 5, 4, 3, 2, or 1 amino acid substitutions in the HC-CDRs; and the VL comprises i) the LC-CDR1 comprising the amino acid sequence of SEQ ID NO: 12, ii) the LC-CDR2 comprising the amino acid sequence of SEQ ID NO: 13, and iii) the LC-CDR3 comprising the amino acid sequence of SEQ ID NO: 14, or a variant thereof comprising 5, 4, 3, 2, or 1 amino acid substitutions in the LC-CDRs;

c) the VH comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, or a variant thereof comprising 5, 4, 3, 2, or 1 amino acid substitutions in the HC-CDRs; and the VL comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22, or a variant thereof comprising 5, 4, 3, 2, or 1 amino acid substitutions in the LC-CDRs;

d) the VH comprises i) the HC-CDR1 comprising the amino acid sequence of SEQ ID NO: 25, ii) the HC-CDR2 comprising the amino acid sequence of SEQ ID NO: 26, and iii) the HC-CDR3 comprising the amino acid sequence of SEQ ID NO: 27, or a variant thereof comprising 5, 4, 3, 2, or 1 amino acid substitutions in the HC-CDRs; and the VL comprises i) the LC-CDR1 comprising the amino acid sequence of SEQ ID NO: 28, ii) the LC-CDR2 comprising the amino acid sequence of SEQ ID NO: 29, and iii) the LC-CDR3 comprising the amino acid sequence of SEQ ID NO: 30, or a variant thereof comprising 5, 4, 3, 2, or 1 amino acid substitutions in the LC-CDRs;

e) the VH comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising 5, 4, 3, 2, or 1 amino acid substitutions in the HC-CDRs; and the VL comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising 5, 4, 3, 2, or 1 amino acid substitutions in the LC-CDRs;

f) the VH comprises i) the HC-CDR1 comprising the amino acid sequence of SEQ ID NO: 41, ii) the HC-CDR2 comprising the amino acid sequence of SEQ ID NO: 42 or 51, and iii) the HC-CDR3 comprising the amino acid sequence of SEQ ID NO: 11; and the VL comprises i) the LC-CDR1 comprising the amino acid sequence of SEQ ID NO: 46, ii) the LC-CDR2 comprising the amino acid sequence of SEQ ID NO: 13, and iii) the LC-CDR3 comprising the amino acid sequence of SEQ ID NO: 47;

h) the VH comprises i) the HC-CDR1 comprising the amino acid sequence of SEQ ID NO: 43, ii) the HC-CDR2 comprising the amino acid sequence of SEQ ID NO: 44 or 52, and iii) the HC-CDR3 comprising the amino acid sequence of SEQ ID NO: 45; and the VL comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 56 or 57; or i) the VH comprises i) the HC-CDR1 comprising the amino acid sequence of SEQ ID NO: 41 or 43, ii) the HC-CDR2 comprising the amino acid sequence of any of SEQ ID NO:58, and iii) the HC-CDR3 comprising the amino acid sequence of SEQ ID NO: 11 or 45; and the VL comprises i) the LC-CDR1 comprising the amino acid sequence of SEQ ID NO: 48, ii) the LC-CDR2 comprising the amino acid sequence of SEQ ID NO: 49, and iii) the LC-CDR3 comprising the amino acid sequence of SEQ ID NO: 50 or 53.

Embodiment 3. The anti-VISTA construct of embodiment 2, wherein the VH comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3; and the VL comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6.

Embodiment 4. The anti-VISTA construct of embodiments 2, wherein the VH comprises i) the HC-CDR1 comprising the amino acid sequence of SEQ ID NO: 9, ii) the HC-CDR2 comprising the amino acid sequence of SEQ ID NO: 10, and iii) the HC-CDR3 comprising the amino acid sequence of SEQ ID NO: 11; and the VL comprises i) the LC-CDR1 comprising the amino acid sequence of SEQ ID NO: 12, ii) the LC-CDR2 comprising the amino acid sequence of SEQ ID NO: 13, and iii) the LC-CDR3 comprising the amino acid sequence of SEQ ID NO: 14.

Embodiment 5. The anti-VISTA construct of embodiment 3, wherein the VH comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19; and the VL comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22.

Embodiment 6. The anti-VISTA construct of embodiment 3, wherein the VH comprises i) the HC-CDR1 comprising the amino acid sequence of SEQ ID NO: 25, ii) the HC-CDR2 comprising the amino acid sequence of SEQ ID NO: 26, and iii) the HC-CDR3 comprising the amino acid sequence of SEQ ID NO: 27; and the VL comprises i) the LC-CDR1 comprising the amino acid sequence of SEQ ID NO: 28, ii) the LC-CDR2 comprising the amino acid sequence of SEQ ID NO: 29, and iii) the LC-CDR3 comprising the amino acid sequence of SEQ ID NO: 30.

Embodiment 7. An anti-VISTA construct comprising an antibody moiety that specifically binds to VISTA, comprising:

a) a HC-CDR1, a HC-CDR2, and a HC-CDR3, respectively comprising the amino acid sequences of a CDR1, a CDR2, and a CDR3 within a VH chain region having the sequence set forth in SEQ ID NO: 7, and a LC-CDR1, a LC-CDR2, and a LC-CDR3, respectively comprising the amino acid sequences of a CDR1, a CDR2, and a CDR3 within a VL chain region having the sequence set forth in SEQ ID NO: 8;

b) a HC-CDR1, a HC-CDR2, and a HC-CDR3, respectively comprising the amino acid sequences of a CDR1, a CDR2, and a CDR3 within a VH chain region having the sequence set forth in SEQ ID NO: 15, and a LC-CDR1, a LC-CDR2, and a LC-CDR3, respectively comprising the amino acid sequences of a CDR1, a CDR2, and a CDR3 within a VL chain region having the sequence set forth in SEQ ID NO: 16;

c) a HC-CDR1, a HC-CDR2, and a HC-CDR3, respectively comprising the amino acid sequences of a CDR1, a CDR2, and a CDR3 within a VH chain region having the sequence set forth in SEQ ID NO: 23, and a LC-CDR1, a LC-CDR2, and a LC-CDR3, respectively comprising the amino acid sequences of a CDR1, a CDR2, and a CDR3 within a VL chain region having the sequence set forth in SEQ ID NO: 24;

d) a HC-CDR1, a HC-CDR2, and a HC-CDR3, respectively comprising the amino acid sequences of a CDR1, a CDR2, and a CDR3 within a VH chain region having the sequence set forth in SEQ ID NO: 31, and a LC-CDR1, a LC-CDR2, and a LC-CDR3, respectively comprising the amino acid sequences of a CDR1, a CDR2, and a CDR3 within a VL chain region having the sequence set forth in SEQ ID NO: 32; or

e) a HC-CDR1, a HC-CDR2, and a HC-CDR3, respectively comprising the amino acid sequences of a CDR1, a CDR2, and a CDR3 within a VH chain region having the sequence set forth in SEQ ID NO: 39, and a LC-CDR1, a LC-CDR2, and a LC-CDR3, respectively comprising the amino acid sequences of a CDR1, a CDR2, and a CDR3 within a VL chain region having the sequence set forth in SEQ ID NO: 40.

Embodiment 8. The anti-VISTA construct of any one of embodiments 1-7, wherein the VH comprises an amino acid sequence of any one of SEQ ID NOs: 7, 15, 23, 31, and 39, or a variant comprising an amino acid sequence having at least about 80% sequence identity; and/or wherein the VL comprises an amino acid sequence of any one of SEQ ID NOs: 8, 16, 24, 32 and 40, or a variant comprising an amino acid sequence having at least about 80% sequence identity.

Embodiment 9. The anti-VISTA construct of embodiment 8, wherein:

a) the VH comprises an amino acid sequence of SEQ ID NO: 7, or a variant comprising an amino acid sequence having at least about 80% sequence identity; and the VL comprises an amino acid sequence of SEQ ID NO: 8, or a variant comprising an amino acid sequence having at least about 80% sequence identity,

b) the VH comprises an amino acid sequence of SEQ ID NO: 15, or a variant comprising an amino acid sequence having at least about 80% sequence identity; and the VL comprises an amino acid sequence of SEQ ID NO: 16, or a variant comprising an amino acid sequence having at least about 80% sequence identity,

c) the VH comprises an amino acid sequence of SEQ ID NO: 23, or a variant comprising an amino acid sequence having at least about 80% sequence identity; and the VL comprises an amino acid sequence of SEQ ID NO: 24, or a variant comprising an amino acid sequence having at least about 80% sequence identity,

d) the VH comprises an amino acid sequence of SEQ ID NO: 31, or a variant comprising an amino acid sequence having at least about 80% sequence identity; and the VL comprises an amino acid sequence of SEQ ID NO: 32, or a variant comprising an amino acid sequence having at least about 80% sequence identity, or

e) the VH comprises an amino acid sequence of SEQ ID NO: 39, or a variant comprising an amino acid sequence having at least about 80% sequence identity; and the VL comprises an amino acid sequence of SEQ ID NO: 40, or a variant comprising an amino acid sequence having at least about 80% sequence identity.

Embodiment 10. The anti-VISTA construct of any one of embodiments 1-9, wherein the antibody moiety is an antibody or antigen-binding fragment thereof selected from the group consisting of a full-length antibody, a bispecific antibody, a single-chain Fv (scFv) fragment, a Fab fragment, a Fab′ fragment, a F(ab′)2, an Fv fragment, a disulfide stabilized Fv fragment (dsFv), a (dsFv)2, a Fv-Fc fusion, a scFv-Fc fusion, a scFv-Fv fusion, a diabody, a tribody, and a tetrabody.

Embodiment 11. The anti-VISTA construct of embodiment 10, wherein the antibody moiety is a full-length antibody.

Embodiment 12. The anti-VISTA construct of any one of embodiments 1-11, wherein the antibody moiety has an Fc fragment is selected from the group consisting of Fc fragments form IgG, IgA, IgD, IgE, IgM, and combinations and hybrids thereof.

Embodiment 13. The anti-VISTA construct of embodiment 12, wherein the Fc fragment is selected from the group consisting of Fc fragments from IgG1, IgG2, IgG3, IgG4, and combinations and hybrids thereof.

Embodiment 14. The anti-VISTA construct of embodiment 12 or embodiment 13, wherein the Fc fragment has a reduced effector function as compared to the corresponding wildtype Fc fragment.

Embodiment 15. The anti-VISTA construct of any one of embodiments 12-14, wherein the Fc fragment has an extended half-life as compared to the corresponding wildtype Fc fragment.

Embodiment 16. The anti-VISTA construct of any one of embodiments 1-15, wherein the antibody moiety of the anti-VISTA construct activates the downstream signaling pathways of VISTA.

Embodiment 17. The anti-VISTA construct of any one of embodiments 1-16, wherein the anti-VISTA construct is an agonist antibody of VISTA.

Embodiment 18. The anti-VISTA construct of any one of embodiment 16, wherein the antibody moiety of the anti-VISTA construct activates or increases the downstream signaling pathways of VISTA by at least about 20%.

Embodiment 19. The anti-VISTA construct of any one of embodiments 1-18, wherein the VISTA is a human VISTA.

Embodiment 20. A pharmaceutical composition comprising the anti-VISTA construct of any one of embodiments 1-19, and a pharmaceutical acceptable carrier.

Embodiment 21. An isolated nucleic acid encoding the anti-VISTA construct of any one of embodiments 1-20.

Embodiment 22. A vector comprising the isolated nucleic acid of embodiment 21.

Embodiment 23. An isolated host cell comprising the isolated nucleic acid of embodiment 21, or the vector of embodiment 22.

Embodiment 24. An immunoconjugate comprising the anti-VISTA construct of any one of embodiments 1-19, linked to a therapeutic agent or a label.

Embodiment 25. A method of producing an anti-VISTA construct comprising:

a) culturing the isolated host cell of embodiment 23 under conditions effective to express the anti-VISTA construct; and

b) obtaining the expressed anti-VISTA construct from the host cell.

Embodiment 26. A method of treating a disease or condition in an individual, comprising administering to the individual an effective mount of the anti-VISTA construct of any one of embodiments 1-19, or the pharmaceutical composition of embodiment 20.

Embodiment 27. The method of embodiment 26, wherein the disease of condition is associated with a dysregulated immune system.

Embodiment 28. The method of embodiment 26 or embodiment 27, wherein the disease or condition is an auto-immune disease, inflammation, an infection, graft versus host disease (GvHD) or a condition associated with a transplant.

Embodiment 29. The method of embodiment 28, wherein the auto-immune disease is selected from cutaneous lupus, rheumatoid arthritis, psoriasis, an autoimmune intestinal disorder, systemic lupus erythematosus (SLE), discoid lupus erythematosus (DLE).

Embodiment 30. The method of any one of embodiments 26-29, wherein the anti-VISTA construct is administered intravenously or subcutaneously into the individual.

Embodiment 31. The method of any one of embodiments, wherein the anti-VISTA construct is administered at a dose of about 0.001 mg/kg to about 100 mg/kg.

Embodiment 32. The method of any one of embodiments 22-31, wherein the individual is a human.

Embodiment 33. A kit comprising any one of the anti-VISTA construct of embodiments 1-19.

### EXAMPLES

The examples below are intended to be purely exemplary of the application and should therefore not be considered to limit the application in any way. The following examples and detailed description are offered by way of illustration and not by way of limitation.

### Example 1. Materials

His-tagged human, mouse, and cynomolgus VISTA extracellular domain proteins were generated in house using standard protocols. hVISTA-mFc was purchased from Adipogen (Cat. # CHI-HF-211B7H5-C100). Alexa Fluor 647 labeled anti-human VISTA antibody (BD Biosciences, Cat #566670) and APC anti-mouse VISTA antibody (Biolegend, Cat. #150205) were used to detect the human and mouse VISTA expressing Jurkat cells.

Jurkat A6 parental cells were transduced with pLenti7.3/TOPO-mouse VISTA or human VISTA full length sequences for binding assays via flow cytometry. Jurkat-nfkb-GFP reporter cells were transduced by lentivirus carrying CMV-Human VISTA extracellular transmembrane together with CD3zeta CAR-EF1-puro for the reporter activation assay via flow cytometry. For each construct, the VISTA positive population of Jurkat cells was sorted twice. Cells for each construct are polyclonal.

### Example 2. Immunizations

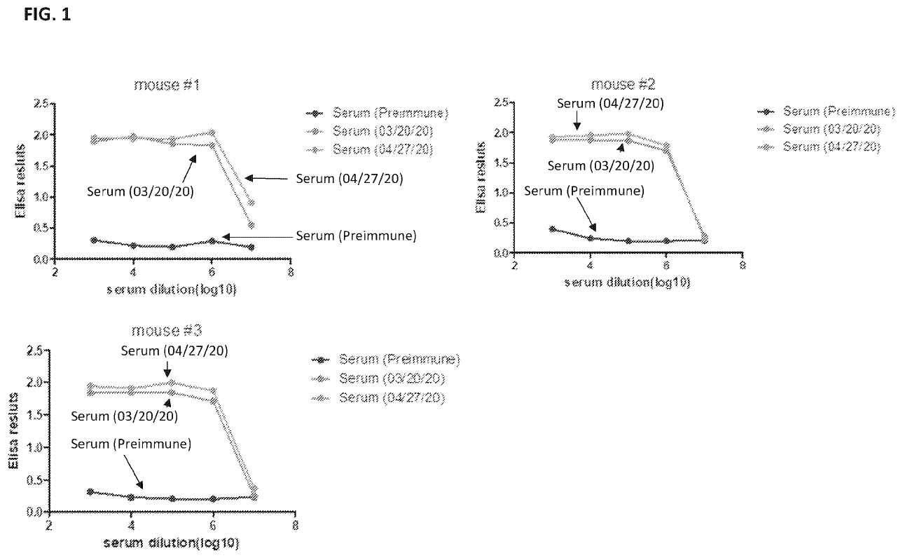

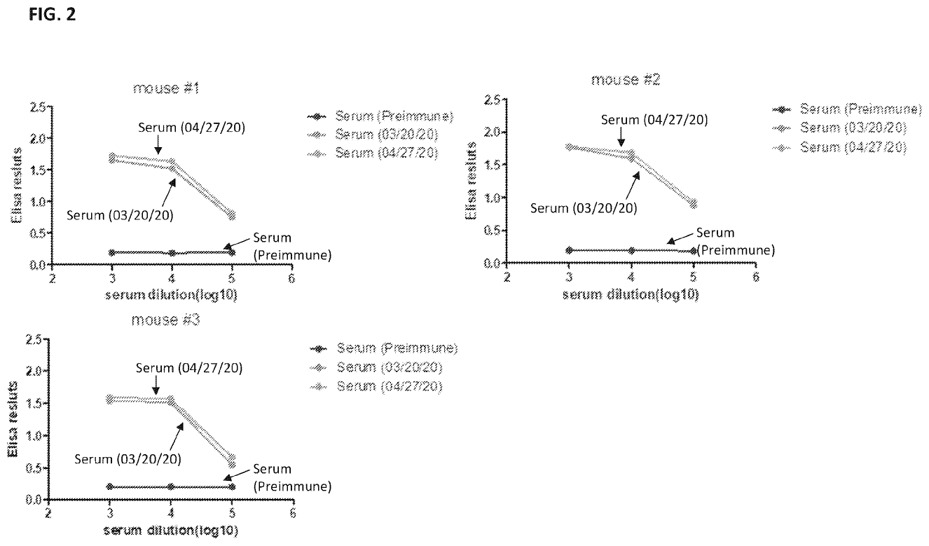

Three VISTA knockout BALB/c mice (female) received 4 immunizations according to the scheme outlined in Table I. The mice were immunized with both hVISTA (human VISTA-mFc) and mVISTA (mouse VISTA-his). The first 3 immunizations included subcutaneous and intraperitoneal injections. The final boost was solely an intraperitoneal injection. The immune responses were analyzed by ELISA: serum samples collected at days 0, day 42 and day 74 were incubated with human VISTA ECD (2 μg/ml) or negative control antibody adsorbed to a 96 well plate. Bound mouse IgG was detected by anti-mouse IgG horseradish peroxidase (Jackson ImmunoResearch, Cat. #615035214). Results are shown in FIGS. 1 and 2.

TABLE 5
Immunization schedule

| | | |
|---|---|---|
| Injections: | 50 ug hVISTA + 50 ug mVISTA + CFA | Day 0 |
| | 50 ug hVISTA + 50 ug mVISTA + IFA | Day 35 |
| | 25 ug hVISTA + 25 ug mVISTA + IFA | Day 67 |
| | 50 ug hVISTA + 50 ug mVISTA + PBS | Day 92 |
| Bleeds: | Pre Bleed | Day 0 |
| | Test Bleed | Day 42 |
| | Test Bleed | Day 74 |
| Spleen Harvest | Cell fusion | Day 97 |

As shown in FIGS. 1 and 2, use of hVISTA or mVISTA produced antibodies that bind to each of the antigens.

### Example 3. Hybridoma Supernatant Production

The spleens from the mice were harvested. Hybridoma cells were prepared following well known procedures. Hybridoma cells were cultured in Hybridoma-SFM media (Gibco) at 37° C. for 7 to 10 days. Cells were pelleted by centrifugation and the supernatants were analyzed as described below in the following Examples.

### Example 4. Primary Screening (Binding to Human VISTA, Mouse VISTA and Cynomolgus VISTA by ELISA)

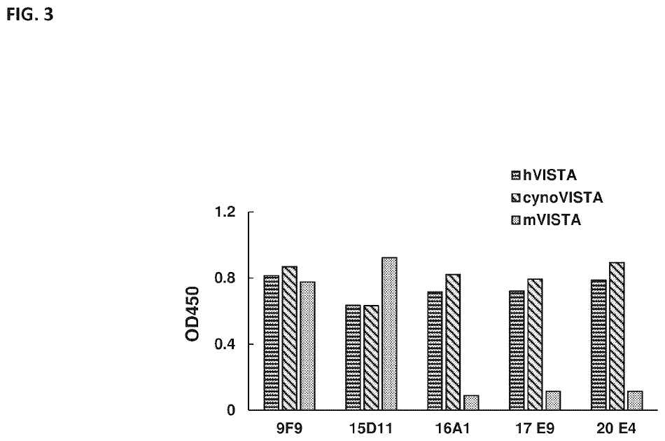

50 μl of hybridoma supernatant was incubated with human VISTA ECD (2 μg/ml), mouse VISTA ECD (2 μg/mL), cynomolgus VISTA (2 μg/mL), or negative mouse IgG1 control coated on 96 well ELISA plates. Mouse anti-VISTA antibodies were detected by anti-mouse IgG-HRP (Jackson ImmunoResearch, Cat. # 615035214). Results are shown in FIG. 3.

As shown in FIG. 3, 9E9, 15D11, 16A1, 17E9, and 20E4 hybridoma supernatants all effectively bind to human, cynomolgus and/or mouse VISTA extracellular domains, and show cross-species reactivity (especially between human and cynomolgus VISTA proteins).

### Example 5. Secondary Screening of Hybridoma Derived Antibodies Binding to VISTA by FACS Assay

Human and mouse VISTA expressing Jurkat cells were cultured in RPMI1640 with 10% FBS. Cells were seeded into a 96 well plate at 1×10⁵ cells/well. Then the cells were incubated with anti-VISTA hybridoma supernatants for 30 minutes at 4° C. 1E8 (Immunext, see WO2017/181139A2) was used as a reference antibody. After washing with FACS buffer, the cells were incubated with Alexa Fluor 647 conjugated anti-mouse IgG(H+L)(1 μg/ml) (Jackson ImmunoResearch, Cat. #61505214) for 30 minutes at 4° C. After washing with FACS buffer twice, the samples were run in a NovoCyte Flow Cytometer (Agilent). Data was analyzed using the NovoExpress Software.

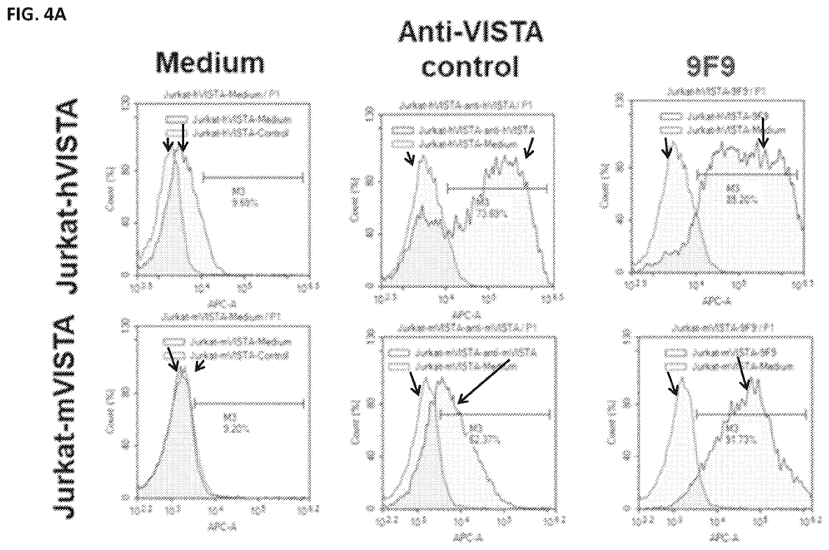

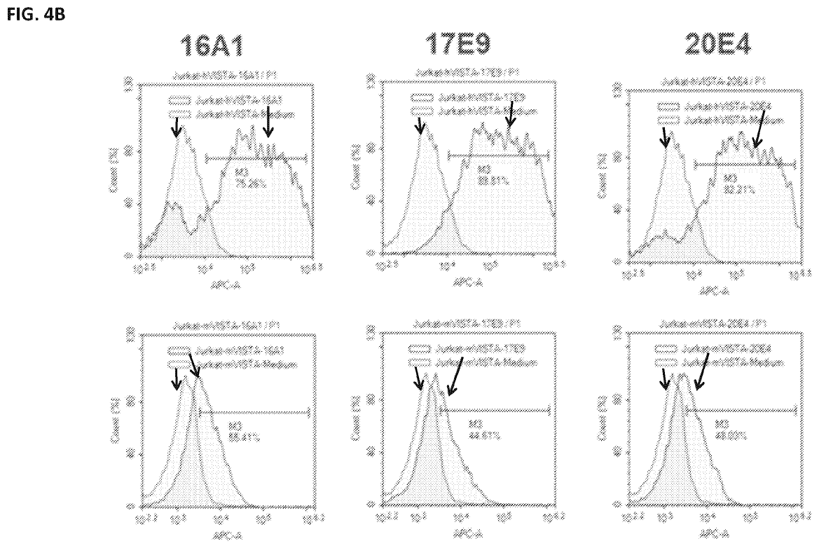

FIGS. 4A-4B show that all anti-VISTA hybridoma tested (9F9, 16A1, 17E9, and 20E4) demonstrate effective binding to Jurkat-hVISTA expressing cell lines, and some binding affinity to Jurkat-mVISTA expressing cell lines.

### Example 6. Reporter Activation Assay of Hybridoma Derived Antibodies

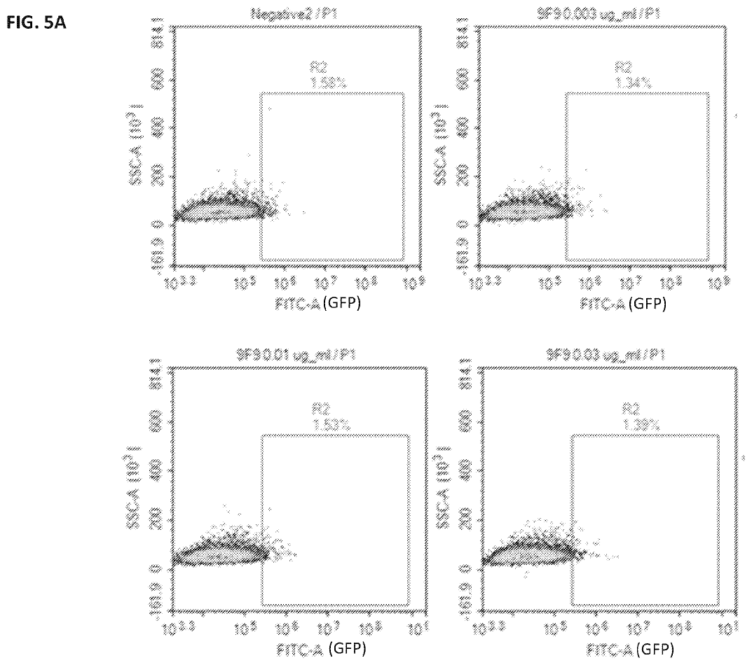

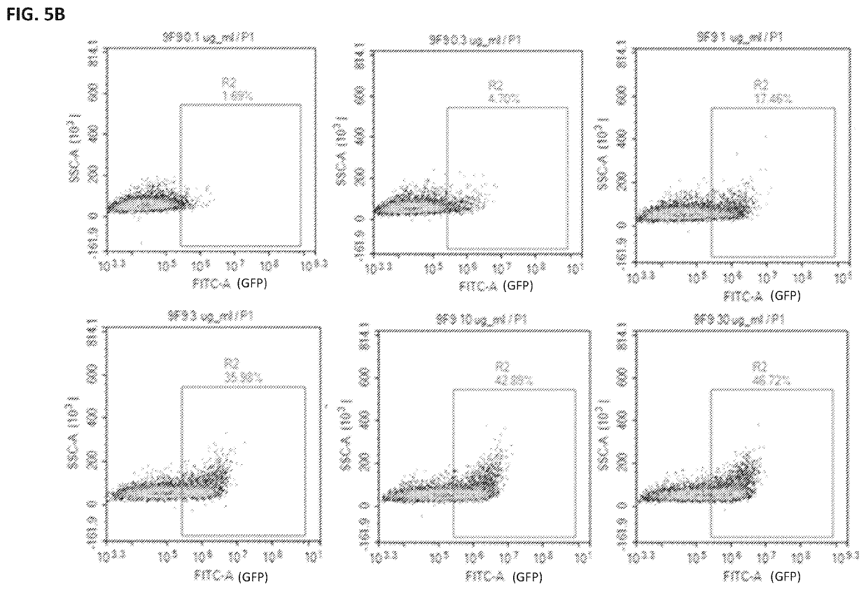

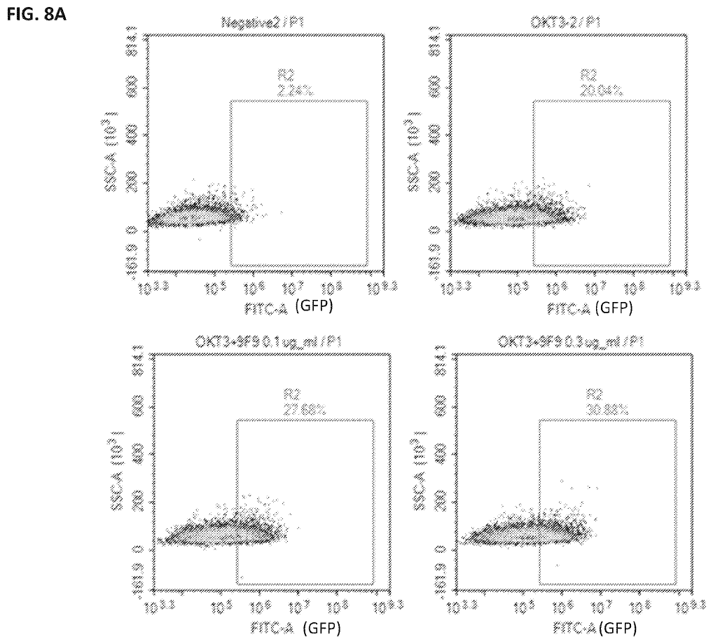

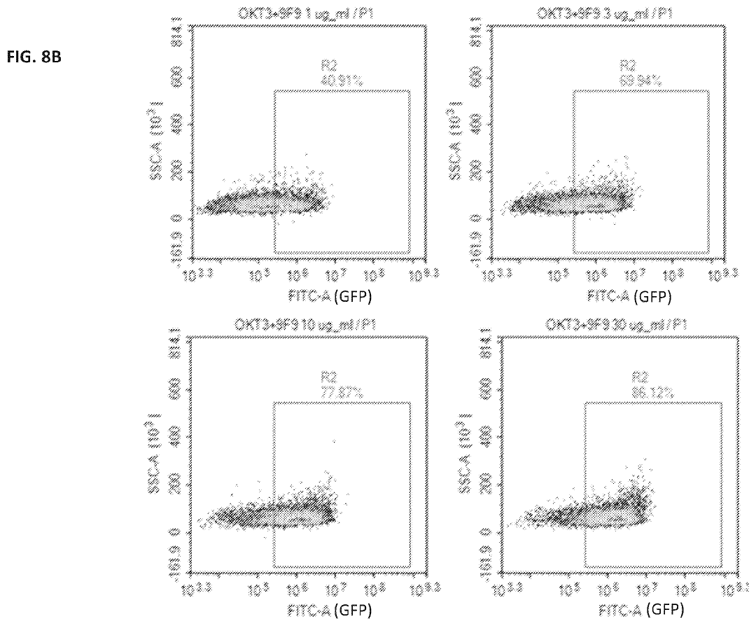

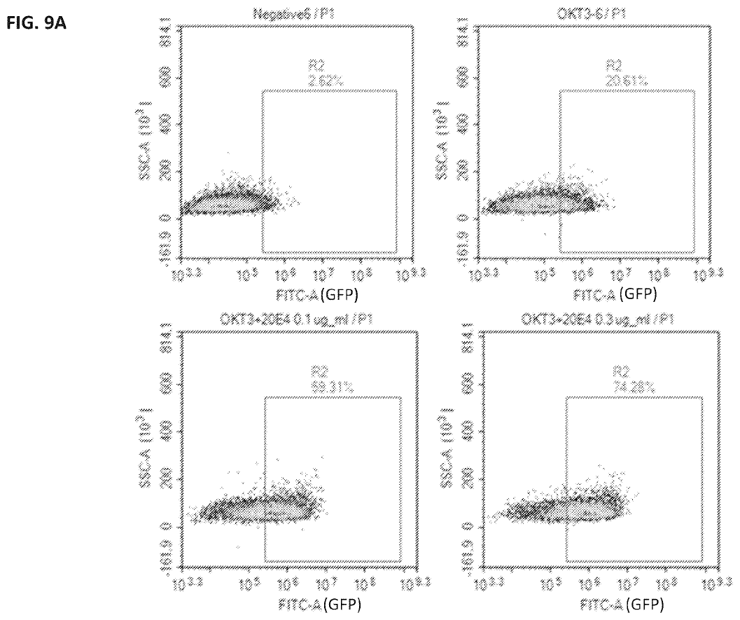

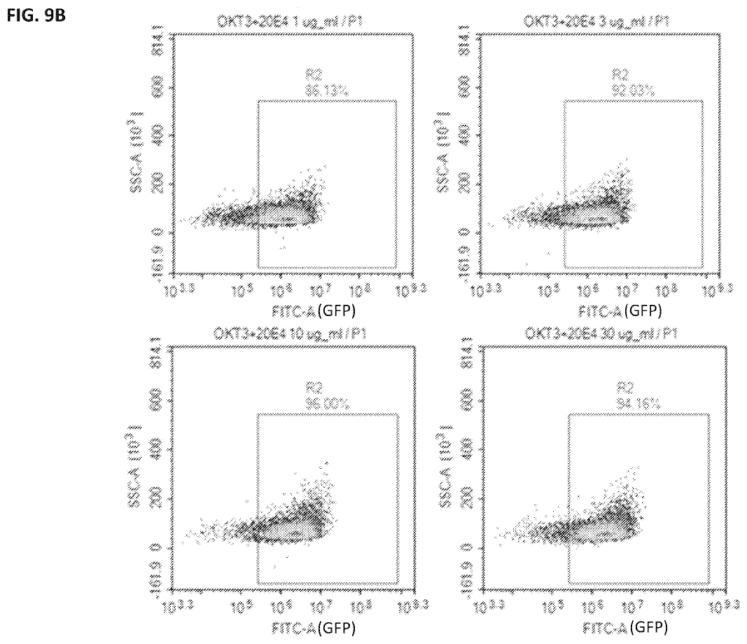

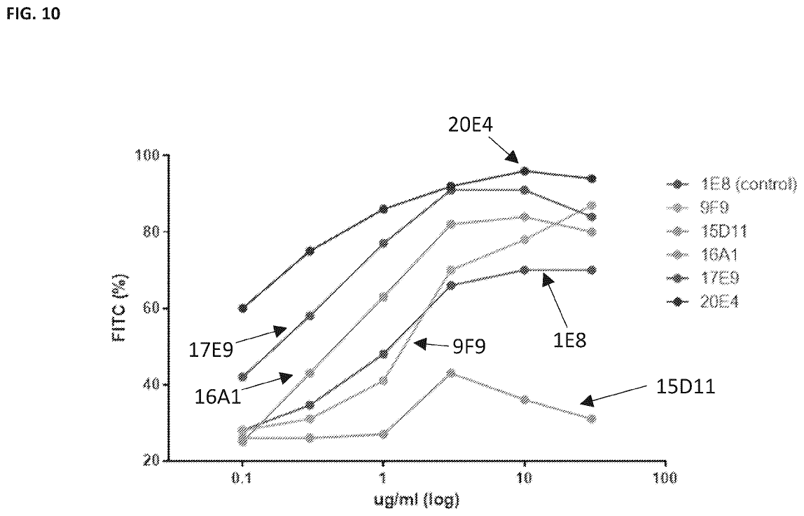

In a 96-well plate, Jurkat-nfkb-GFP/human VISTA-hCD3z cells were seeded at 1×10⁵ cells per well, in 200 μl RPMI1640 containing 10% FBS medium. Hybridoma supernatants were added at increasing anti-VISTA concentrations to the medium (0.003, 0.01, 0.03, 0.1, 0.3, 1, 10, and 30 μg/ml based on ELISA). GFP was measured by flow cytometry after 24 hours incubation. Results are shown in FIGS. 5-7. To test the effect of OKT3 on cell activation, OKT3 (3 ng/ml) was added to the cells in the presence of increasing anti-VISTA concentrations from hybridoma derived antibodies for the 24 hr. incubation. Results are shown in FIGS. 8A-10.

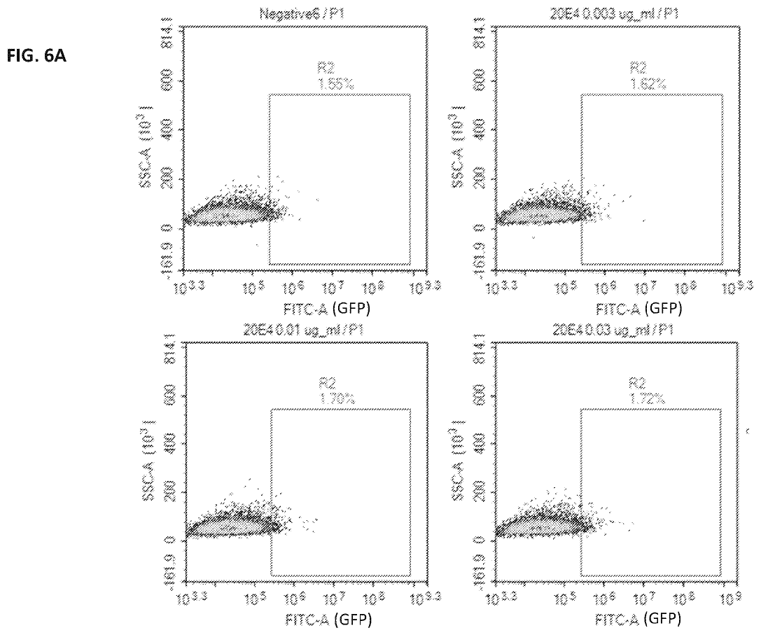

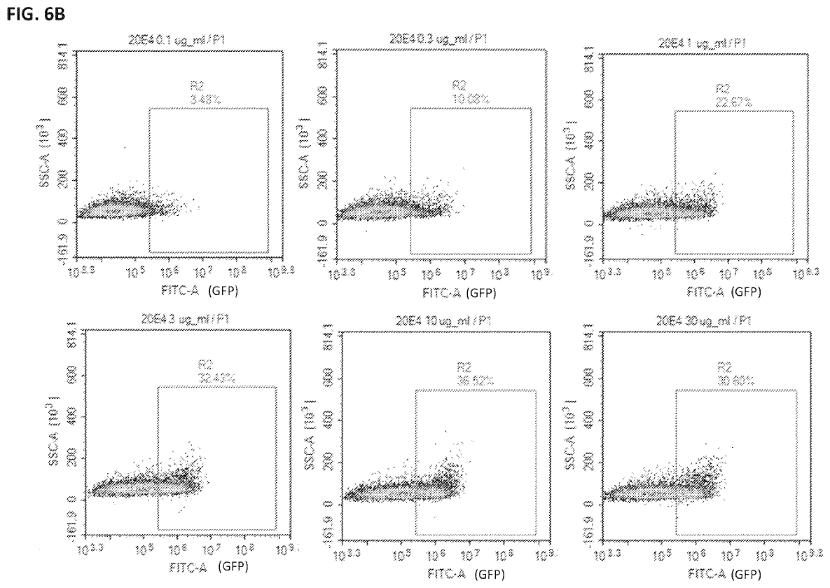

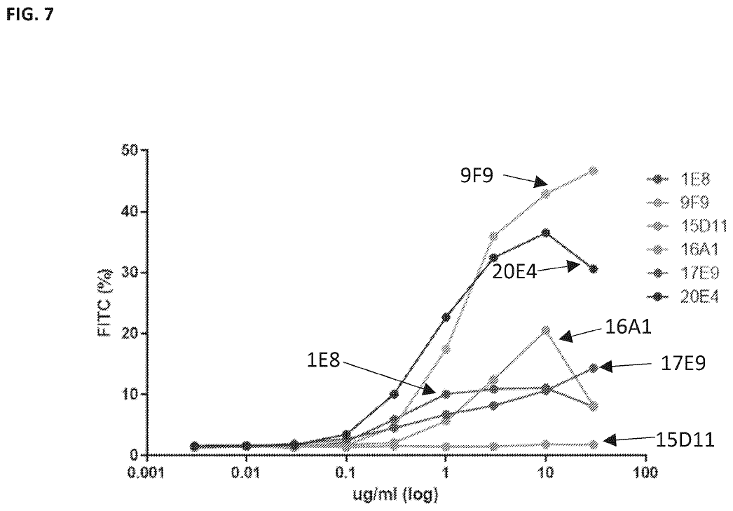

FIGS. 5A-5B and 6A-6B show that upon the incubation with 9F9 or 20E4 hybridoma supernatant at various concentrations, VISTA downstream pathway was activated in Jurkat-NFKb-GFP/hVISTA-hCD3z expressing cells, as indicated by positive GFP staining. The extent of activation is in a concentration-dependent manner. 16A1, 17E9, and 1E8 also exhibit the ability to activate Jurkat-NFKb-GFP/hVISTA-hCD3z cells at certain concentrations. See FIG. 7. Accordingly, 9F9, 20E4, 1E8, 16A1, and 17E9 all exhibit agonistic effect on human VISTA, with 9F9 and 20E4 exhibit highest agonistic effect among these antibodies.

FIGS. 8A-8B, 9A-9B, and 10 show that OKT3 significantly increased the percentage of activated Jurkat-NFKb-GFP/hVISTA-hCD3z cells. The percentages of activated cells reached more than 60% for all antibodies tested except 15D11. For cells incubated with 20E4 and 17E9, or 16A1, more than 80% of the cells were activated at certain concentrations. These results show the strong agonistic effects of these antibodies.

### Example 7. Sequence Analysis

Total RNA was isolated from the hybridoma cell line culture. RNA was treated to remove aberrant transcripts and reverse transcribed using oligo(dT) primers. Samples of the resulting cDNA were amplified in separate PCRs using framework 1 and constant region primer pairs specific for either the heavy or light chain. Reaction products were separated on an agarose gel, size evaluated, and recovered. Amplicons were cloned into the pUC19 vector (Clontech). Ten colonies were selected and both plasmid DNA and PCR products were subjected to Sanger sequencing.

DNA sequence data from all constructs were analyzed and consensus sequences for heavy and light chain were determined. See Tables 3 and 4 below.

TABLE 3
VH CDR sequences and consensus sequences

| | HC-CDR1 | HC-CDR2 | HC-CDR3 |
|---|---|---|---|
| 9F9 | GYWMQ | AIYPGDGDTRYSQKFKG | RDYGAWFAY |
| | (SEQ ID NO: 1) | (SEQ ID NO: 2) | (SEQ ID NO: 3) |
| 20E4 | SSWVE | EIFPGSGSTHYNEKFKG | RPPGWFFDV |
| | (SEQ ID NO: 9) | (SEQ ID NO: 10) | (SEQ ID NO: 11) |
| 16A1 | SSWIE | EIFPGSGSLNFNEKFKG | RPPGWFFDV |
| | (SEQ ID NO: 17) | (SEQ ID NO: 18) | (SEQ ID NO: 19) |
| 17E9 | SCWIE | EILPGSDYTNYNEKFKD | RPPGWYFDV |
| | (SEQ ID NO: 25) | (SEQ ID NO: 26) | (SEQ ID NO: 27) |
| 15D11 | DTFIH | RIDPANGNSKYDPKFQG | WPSNWEAMDY |
| | (SEQ ID NO: 33) | (SEQ ID NO: 34) | (SEQ ID NO: 35) |
| Consensus | SSWX₁E | EIFPGSGSX₂X₃X₄NEKFKG | RPPGWFFDV |
| sequence | X₁ = V or I | X₂ = T or L, | (SEQ ID NO: 11) |
| Based on | (SEQ ID NO: 41) | X₃ = H or N, | |
| 20E4 and | | X₄ = Y or F | |
| 16A1 | | (SEQ ID NO: 42) | |
| | | EIFPGSGSX₂X₃X₄NEKFKG | |
| | | X₂X₃X₄ = THY or LNF | |
| | | (SEQ ID NO: 51) | |
| Consensus | SX₁WX₂E | EIX₃PGSX₄X₅TX₆YNEKFKX₇ | RPPGWX₈FDV |
| sequence | X₁ = S or C, | X₃ = F or L, | X₈ = F or Y |
| Based on | X₂ = V or I | X₄ = G or D, | (SEQ ID NO: 45) |
| 20E4 and | (SEQ ID NO: 43) | X₅ = Y or S, | |
| 17E9 | | X₆ = H or N, | |
| | | X₇ = G or D | |
| | | (SEQ ID NO: 44) | |
| | | EIX₃PGSX₄X₅TX₆YNEKFKX₇ | |
| | | X₃ = F or L, | |
| | | X₄X₅ = DY or GS, | |
| | | X₆ = H or N, | |
| | | X₇ = G or D | |
| | | (SEQ ID NO: 52) | |
| Consensus | SX₁WX₂E | EIX₃PGSX₄X₅X₆X₇X₈NEKFKX₉ | RPPGWX₈FDV |
| sequence | X₁ = S or C, | X₃ = F or L, | X₈ = F or Y |
| based on | X₂ = V or I | X₄X₅X₆X₇X₈ = GSTHY, | (SEQ ID NO: 45) |
| 20E4, | (SEQ ID NO: 43) | GSLNF, or DYTNY, | |
| 16A1 and | | X₉ = F or L | |
| 17E9 | | (SEQ ID NO: 58) | |

TABLE 4
VL CDR sequences and consensus sequences

| | LC-CDR1 | LC-CDR2 | LC-CDR3 |
|---|---|---|---|
| 9F9 | KASQDINSYLS | RANRLVD | LQYDEFPYT |
| | (SEQ ID NO: 4) | (SEQ ID NO: 5) | (SEQ ID NO: 6) |
| 20E4 | KASQDVSTDVA | SASYRYT | QQHYSTPWT |
| | (SEQ ID NO: 12) | (SEQ ID NO: 13) | (SEQ ID NO: 14) |
| 16A1 | KASQDVTTDVA | SASYRYT | QQHYSSPWT |
| | (SEQ ID NO: 20) | (SEQ ID NO: 21) | (SEQ ID NO: 22) |
| 17E9 | KASQDVSTDIA | SASYRFT | QHQYSTPWT |
| | (SEQ ID NO: 28) | (SEQ ID NO: 29) | (SEQ ID NO: 30) |
| 15D11 | SASSSVSYMY | DTSNLAS | QQWISYPLT |
| | (SEQ ID NO: 36) | (SEQ ID NO: 37) | (SEQ ID NO: 38) |
| Consensus | KASQDVX₁TDVA | SASYRYT | QQHYSX₂PWT |
| sequence | X₁ = S or T | (SEQ ID NO: 13) | X₂ = S or T |
| Based on | (SEQ ID NO: 46) | | (SEQ ID NO: 47) |
| 20E4 and | | | |
| 16A1 | | | |
| Consensus | KASQDVSTDX₁A | SASYRX₂T | QX₃X₄YSTPWT |
| sequence | X₁ = V or I | X₂ = Y or F | X₄ = Q or H, |
| Based on | (SEQ ID NO: 54) | (SEQ ID NO: 55) | X₅ = Q or H |
| 20E4 and | | | (SEQ ID NO: 56) |
| 17E9 | | | QX₃X₄YSTPWT |
| | | | X₄X₅ = QH or HQ |
| | | | (SEQ ID NO: 57) |
| Consensus | KASQDVX₁TDX₂A | SASYRX₃T | QX₄X₅YSX₆PWT |
| sequence | X₁ = S or T, | X₃ = Y or F | X₄ = Q or H, |
| Based on | X₂ = V or I | (SEQ ID NO: 49) | X₅ = Q or H, |
| 20E4, | (SEQ ID NO: 48) | | X₆ = S or T |
| 16A1 and | | | (SEQ ID NO: 50) |
| 17E9 | | | QX₄X₅YSX₆PWT |
| | | | X₄X₅ = QH or HQ, |
| | | | X₆ = S or T |
| | | | (SEQ ID NO: 53) |

### Example 8. Anti-VISTA Antibody Expression and Purification from Hybridoma Cells

Hybridoma cells were cultured in Hybridoma-SFM media (Gibco) at 37° C. for 7 to 10 days. Cells were pelleted by centrifugation and the supernatants were collected for antibody purification using Protein G Sepharose (GE). The elution from the column was buffer exchanged to 1XPBS (pH 7.4) by diafiltration via centrifugation. The purified antibodies were analyzed by SDS-PAGE gel electrophoresis to confirm the purity and size. The concentration of the antibody was determined by Also on a spectrophotometer. The purified antibodies were 0.2 μm filtered prior to storage.

### Example 9. Recombinant Expression and Purification of nti-VISTA antibodies in Expi293 Cells

Anti-VISTA variable domains were cloned with a mIgG1 constant domain and expressed in Expi293 cells. Briefly, the mouse IgG1 variable domains were gene synthesized using human preferred codons from IDT. Then the gene fragments were subcloned into the pcDNA3.4 vector which contains the murine antibody signal sequences and mIgG1 Fc fragment. The antibodies were produced by transient transfection into Expi293 cells using the ExpiFectamine 293 transfection kit (Thermo Fisher Scientific). Five days after transfection, the supernatants from transfected cells were collected and purified using Protein G Sepharose (GE). The bound antibodies were eluted using 0.1M Glycine buffer (pH 2.7) and dialyzed with 1XPBS (pH 7.4) overnight. The purified antibodies were analyzed on reduced and non-reduced SDS-PAGE to confirm the purity and size. Recombinant protein concentration was determined by A₂₈₀ on a spectrophotometer.

### Example 10. Binding of Recombinant Anti-VISTA Antibodies to Cell Surface Expressing Human and mouse VISTA Jurkat Cells Determined by Fluorescence Activated Cell Sorting (FACS) assay

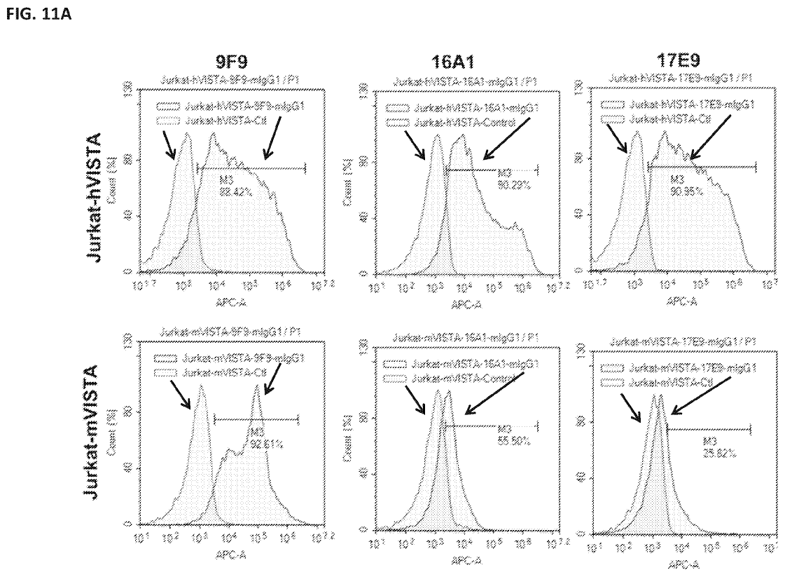

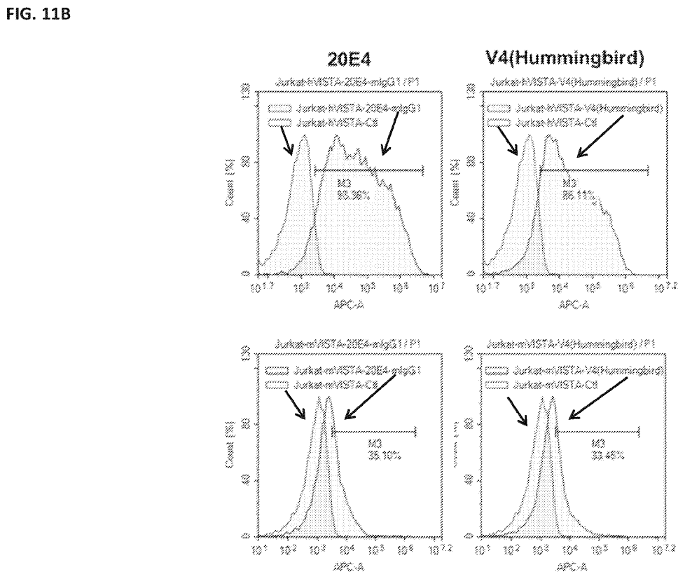

Human and mouse expressing Jurkat cells were cultured in RPMI1640 containing 10% FBS. Cells were seeded into a 96 well plate at 1×10⁵ cells/well. Then the cells were incubated with recombinant anti-VISTA antibody 9F9, 16A1, 17E9, 20E4 or V4 (Hummingbird) (10 μg/ml) for 30 minutes at 4° C. After washing with FACS buffer, the cells were incubated with Alexa Fluor 647 conjugated anti-mouse IgG(H+L)(1 μg/ml) (Jackson ImmunoResearch, Cat. #615605214) 30 minutes at 4° C. Cells were washed with FACS buffer twice, and the samples were run in a NovoCyte Flow Cytometer (Agilent). Data was analyzed using the NovoExpress Software. Results are shown in FIGS. 11A-11B.

FIGS. 11A-11B demonstrate that recombinant mIgG1 anti-VISTA antibodies (9F9, 16A1, 17E9, and 20E4) all show effective binding to Jurkat-hVISTA expressing cell lines. In addition, 9F9 also shows effective binding to Jurkat-mVISTA, indicating cross-species reactivity.

### Example 11. Reporter Activation Assay of Recombinant Anti-VISTA Antibodies

In a 96-well plate, Jurkat-nfkb-GFP/human VISTA-hCD3z cells were seeded at lx10 5 cells per well, in 200 ill RPMI1640 containing 10% FBS medium. Recombinant anti-VISTA antibodies were added at increasing VISTA concentrations to the medium (0.003, 0.01, 0.03, 0.1, 0.3, 1, 10, and 30 μg/ml) or (0.05,0.5,5 and 50 μg/ml). GFP was measured by flow cytometry after a 24 hr. incubation. To test the effect of OKT3 on cell activation, OKT3 (3 ng/ml) was added to the cells in the presence of increasing anti-VISTA concentrations and GFP was measured after the 24 hr incubation.

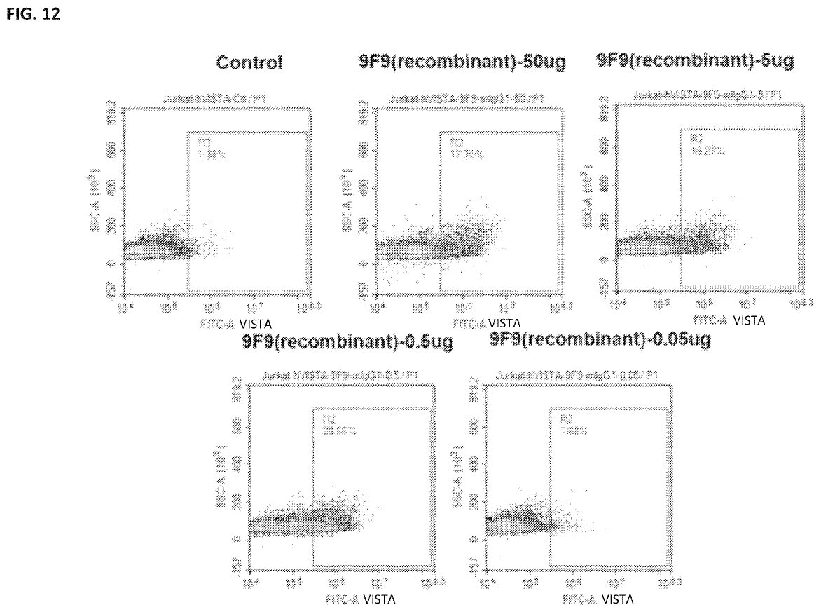

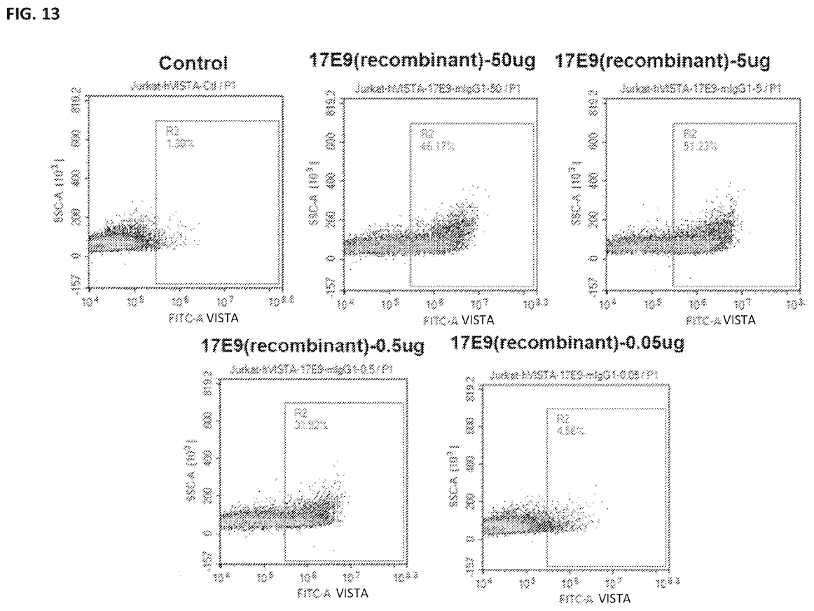

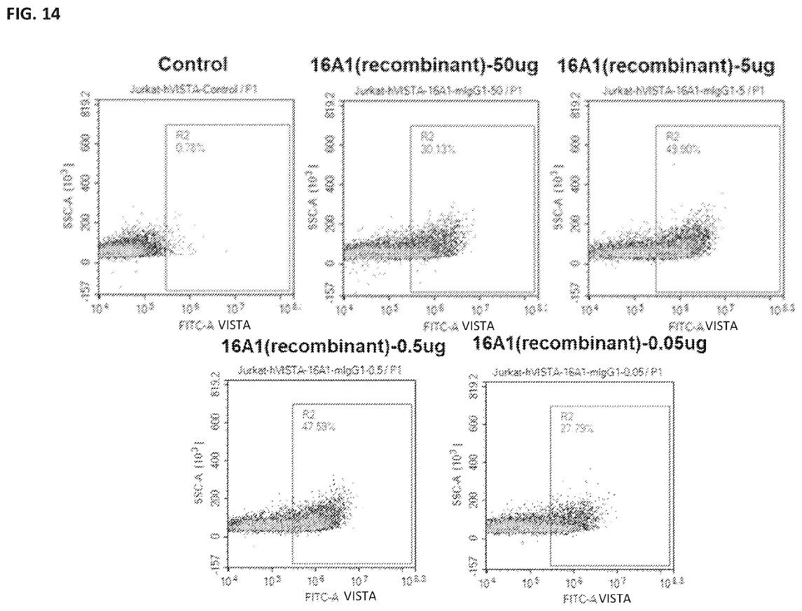

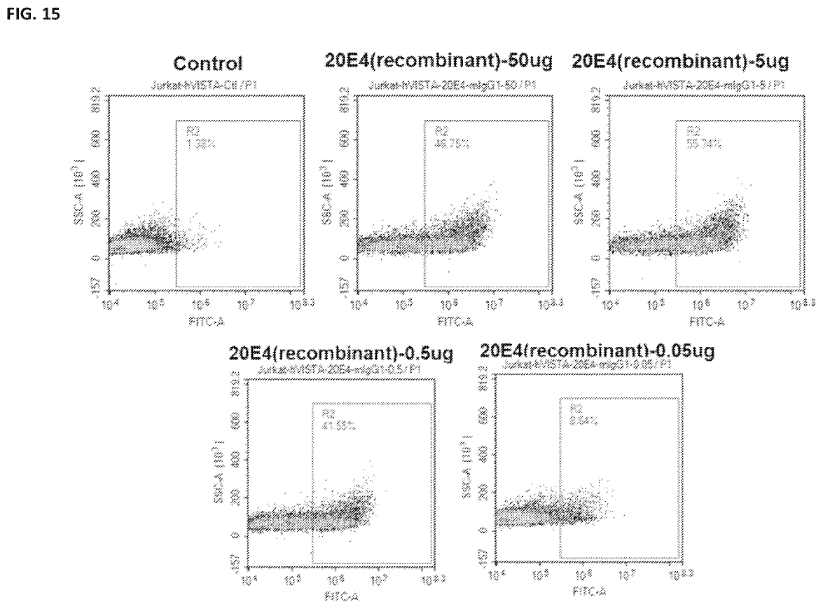

FIGS. 12-15 show that recombinant mIgG1 anti-VISTA antibodies (9F9, 16A1, 17E9, and 20E4) induce effective activation in Jurkat-NFKb-GFP/hVISTA-hCD3z expressing cell lines, with 15%-50% cells activated at a 5 μg/mL concentration.

### Example 12. Epitope Binning Assay of Anti-VISTA Antibodies by Octet Competition

Anti-VISTA antibody epitope bins were determined using Octet QKe (ForteBio). Human VISTA recombinant protein (Sino Biological Inc., Cat. #13485-H08H) was biotinylated using EZ-LINK NHS-PEG4 biotin (Thermo Fisher Scientific). Streptavidin biosensors tips (ForteBio) were used to capture the biotinylated VISTA protein (300 second at 5 μg/ml). A baseline measurement was stabilized for 60 seconds in 1× kinetics buffer (Fortebio) before primary anti-VISTA antibodies (10 μg/ml) were allowed to associate for 300 seconds with captured protein. A panel of secondary anti-VISTA antibodies (10 μg/ml) were then allowed to associate with the antigen and primary antibody complex for an additional 300 seconds. Signals were recorded for each binding event and data analysis was performed on ForteBio Data Analysis HT 11.1 software.

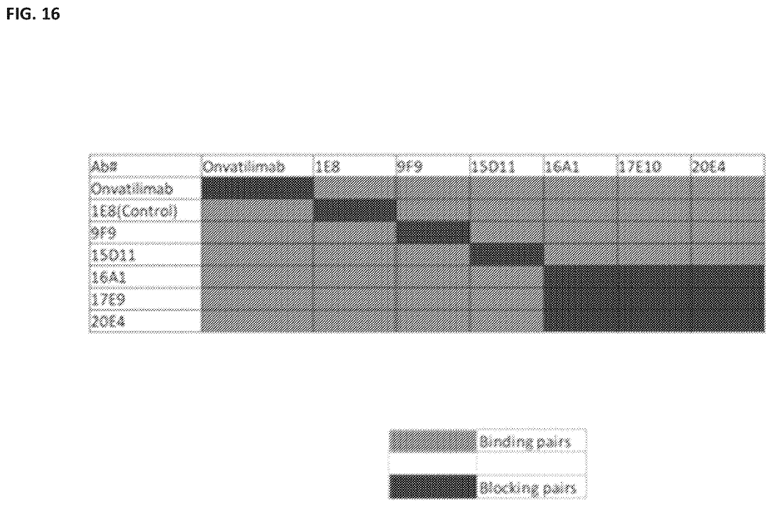

The Epitope binning assay of lead anti-VISTA antibodies has showed three group binding epitopes in this Octet analysis. FIG. 16 show that 16A1, 17E9 and 20E4 compete for the same epitope on VISTA, while 1E8, 9F9, and 15D11 bind to different epitopes. The binding epitopes from all these antibodies bind to are different from that of the benchmark control antibodies Onvatilimab and 1E8 (Immunext).

### Example 13. Human, Cynomolgus and Mouse VISTA Antigen Cross-binding Activities of Anti-VISTA mAbs by Bio-layer Interferometry (BLI) Assay

The human, cynomolgus and mouse VISTA antigen cross-binding activities of anti-VISTA antibodies were determined with bio-layer interferometry using Octet QKe (ForteBio). Human VISTA recombinant protein (Sino Biological Inc., Cat. #13482-H08H) mouse VISTA (Sino Biological Inc., Cat. #51550-MO8H) or cynomolgus VISTA protein (made in house) were biotinylated using EZ-LINK NHS-PEG4 biotin (Thermo Fisher Scientific). Streptavidin biosensors (ForteBio) were used to load biotinylated VISTA protein (300 seconds at 5 μg/ml). The baseline measurement was stabilized for 60 seconds in 1× kinetics buffer (ForteBio) before anti-VISTA antibodies at 5μg/m1 were allowed to associate for 300 seconds with captured protein. Then the sensors were dissociated in 1× kinetics buffer for 600 seconds. Data analysis was performed on ForteBio Data Analysis HT 11.1 software.

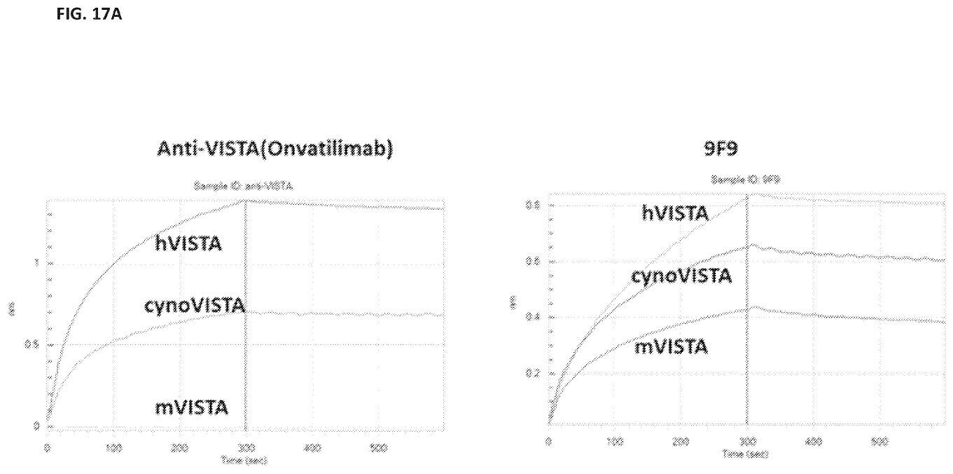

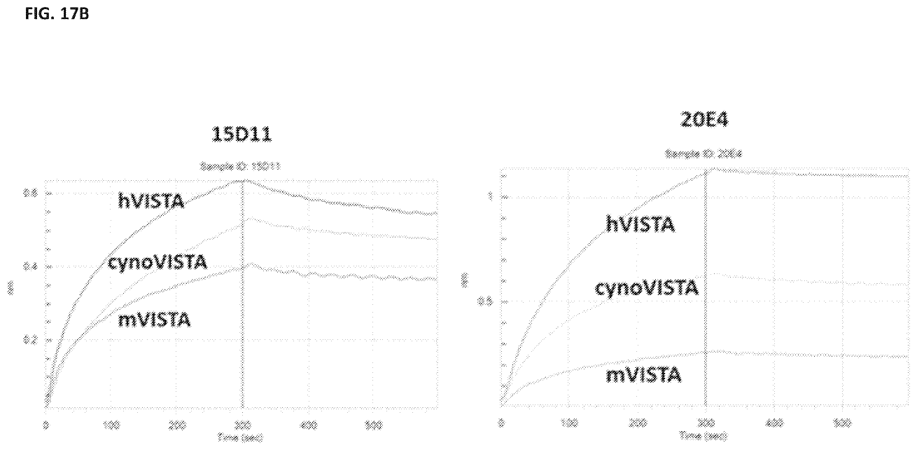

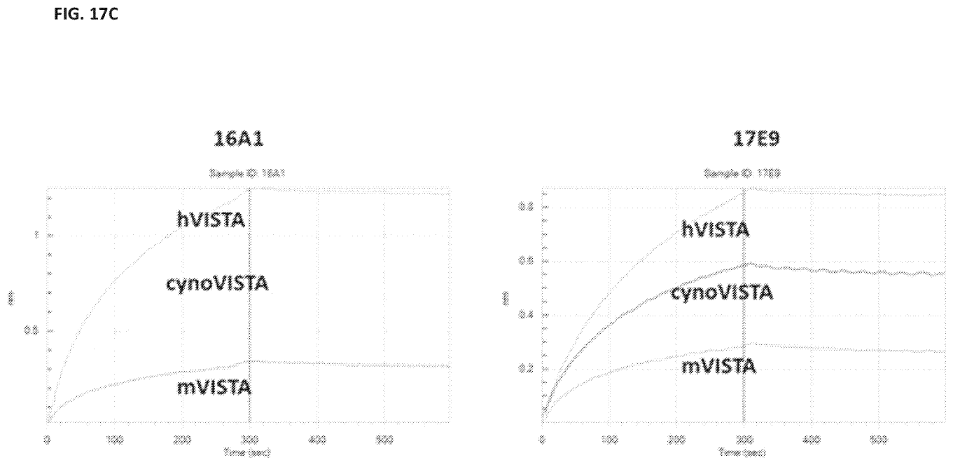

FIGS. 17A-17C show that all antibodies tested demonstrate effective binding to human, cynomolgus monkey, and mouse VISTA. In comparison, the benchmark control antibody Onvatilimab does not cross-react with mouse VISTA.

### Example 14. Binding Affinity of Anti-VISTA Antibodies for Human and Cynomolgus VISTA Determined by Bio-layer Interferometry (BLI) assay

The binding affinities of anti-VISTA antibodies for human and cynomolgus were determined with bio-layer interferometry using Octet QKe (ForteBio). Human VISTA recombinant protein (Sino Biological Inc., Cat. #13482-H08H) or cynomolgus VISTA protein (made in house) were biotinylated using EZ-LINK NHS-PEG4 biotin (Thermo Fisher Scientific). Streptavidin biosensors (ForteBio) were used to load biotinylated VISTA protein (300 seconds at 5 μg/ml). The baseline measurement was stabilized for 60 seconds in 1X kinetics buffer (ForteBio) before anti-VISTA antibodies at a serial dilution (50, 25, 12.5, 6.25 and 3.125 μg/ml) were allowed to associate for 300 seconds with captured protein. Then the sensors were dissociated in 1× kinetics buffer for 600 seconds. Data analysis was performed on ForteBio Data Analysis HT 11.1 software.

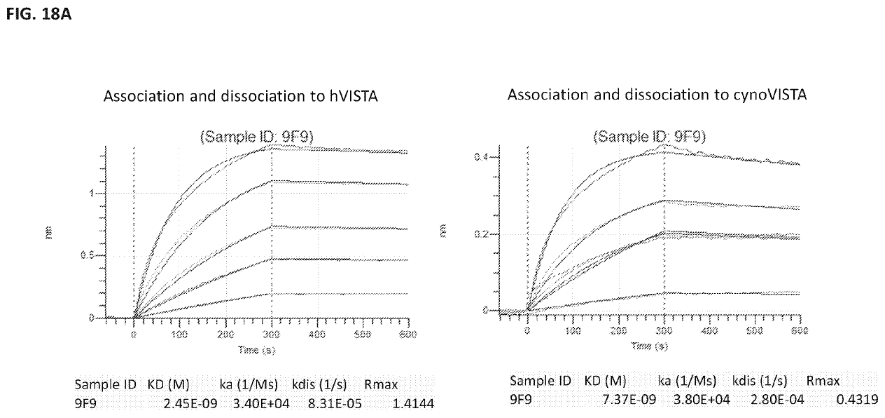

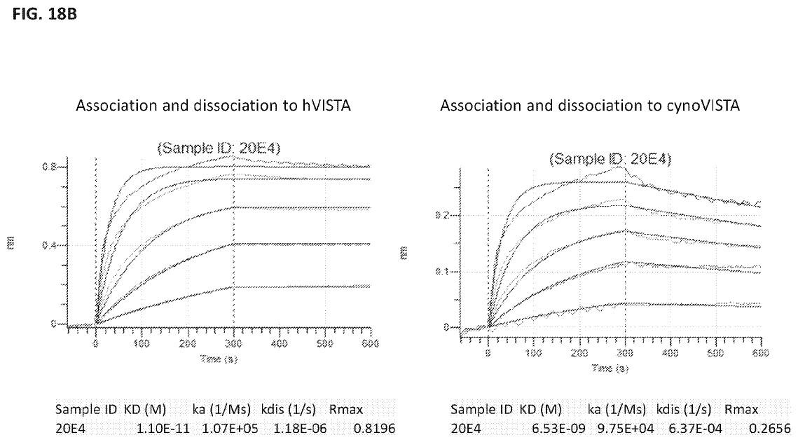

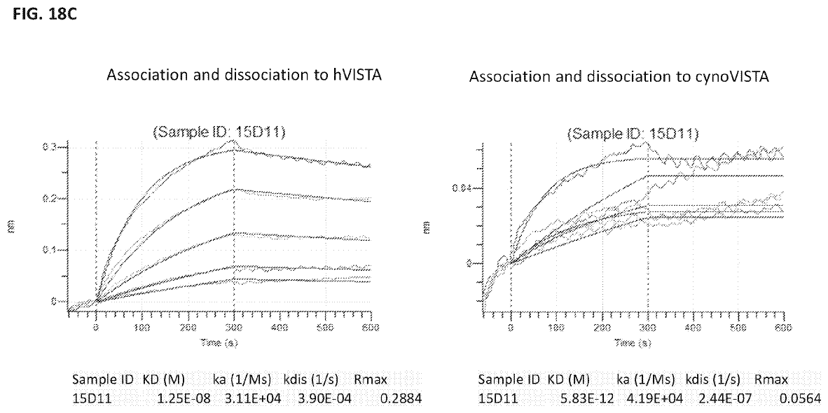

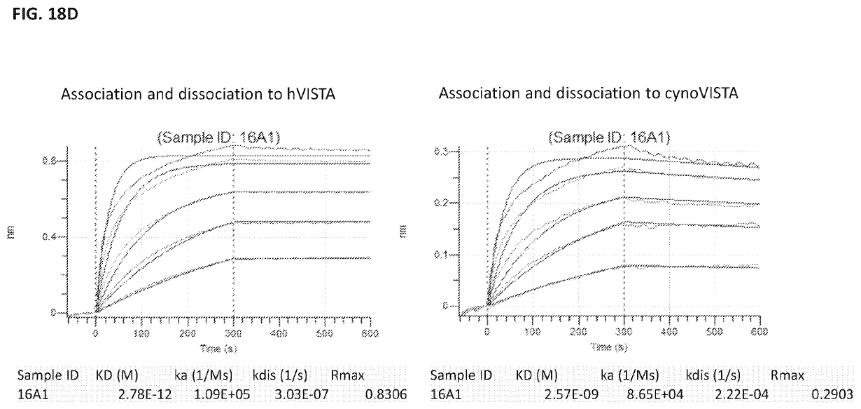

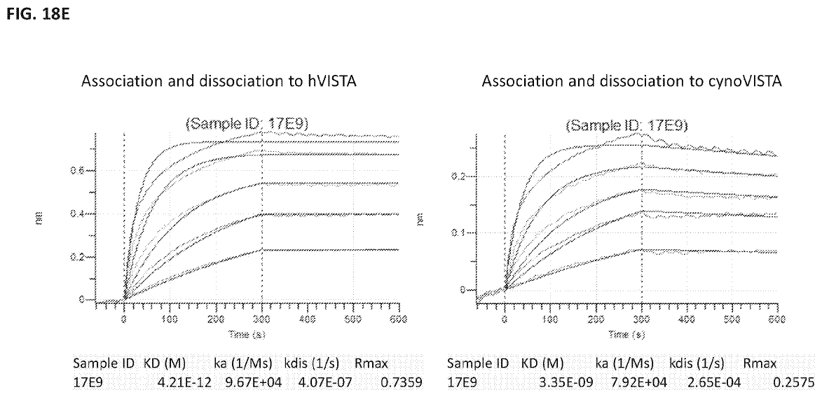

FIGS. 18A-18E show the binding affinities of all anti-VISTA antibodies to human and cynomolgus monkey VISTA.

### Example 15. Analysis of the Ability of Anti-VISTA Antibodies for Inhibition of T Cell Proliferation

The ability of anti-VISTA antibodies to inhibit T cell proliferation was analyzed in an in vitro assay. Anti-CD3 (OKT3) and VISTA-Ig at a concentration ratio of 1:1(2.5 μg/ml anti-CD3: 2.5 μg/ml) were coated to 96-well overnight at 37° C. Human PBMCs were purified from freshly collected buffy coats and labeled with CFSE (Invitrogen, Cat#C34554). Then the 96-well plate wells were washed three times with 1XPBS buffer and 200,000 CFSE-labelled PBMC cells were added to each well with CTS OpTmizer T cell expansion SFM (Invitrogen, Cat#A1048501) and in the presence of anti-VISTA antibodies (50 μg/ml) or mouse IgG control (50 μg/ml). After 5-day treatment, cells were harvested, labelled with APC conjugated anti-human CD3 antibody (Biolegend Cat#300311) and run in a NovoCyte Flow Cytometer (Agilent). Data was analyzed using the NovoExpress Software. Results are shown in FIG. 19.

The benchmark control antibody 1E8 (Immunext) was used as a positive control and mouse IgG isotype was used as a negative control. As shown, about 50%-70% cells incubated with OKT3 only, OKT3+VISTA-Ig, or OKT3+VISTA-Ig+mIgG proliferated as indicated by CFSE staining. In contrast, the presence of an anti-VISTA antibody (1E8, 9F9, 15D11, 16A1, 17E9, and 20E4) significantly suppressed the T cell proliferation. Among these, 9F9, 15D11, 16A1 and 20E4 exhibit better suppression effect than 1E8.

### Example 16. Animal Study

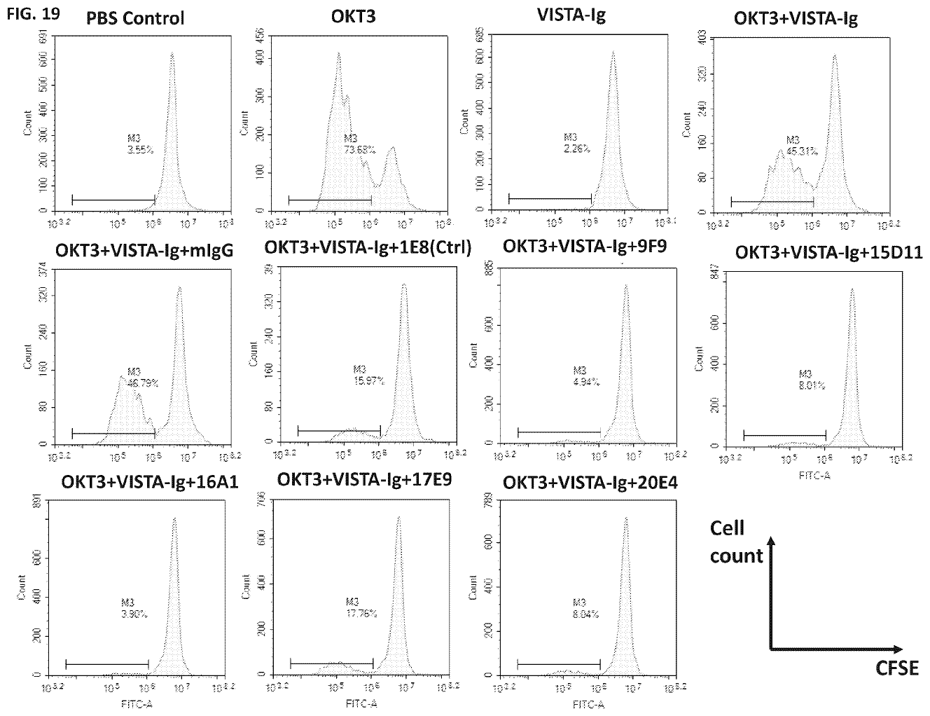

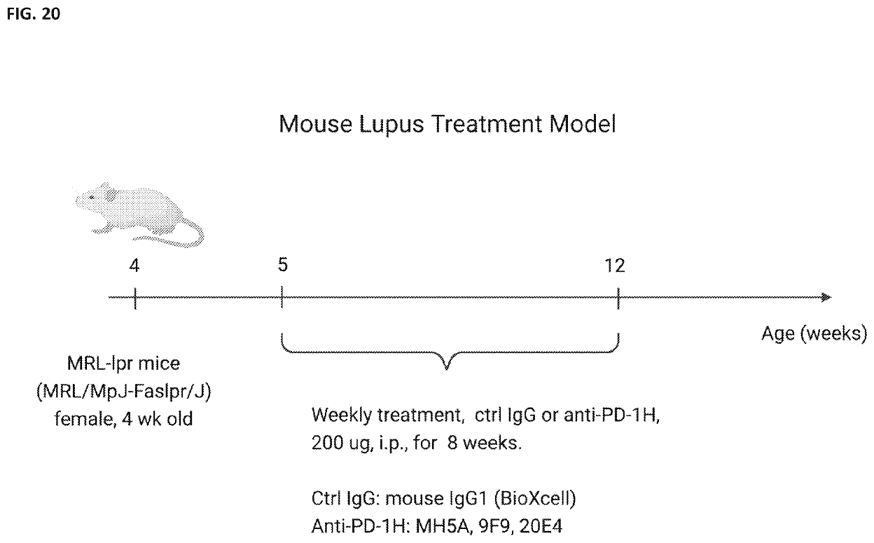

Female MRL-lpr mice of 4-week old were used in lupus treatment model. During weeks 5-12, different groups of mice were given weekly treatment of 200 μg mIgG1 (control) or anti-VISTA constructs (MHSA, 9F9, or 20E4). Most measurements were taken on or after week 12, with several measurements of serum antibody levels taken before week 12. A graphic summary of the protocol used for lupus treatment model is shown in FIG. 20. Experiments A-F below were designed using major clinical indicators of lupus in examining the efficacy of anti-VISTA constructs tested herein.

### A. Anti-VISTA Constructs in Treating Lymph Node Enlargement

MRL-lpr mice show massive lymphadenopathy associated with proliferation of aberrant T cells caused by spontaneous mutation Fas^{lpr}. This autoimmune mouse model develops skin lesions and is commonly used as a model for lupus erythematosis. Enlarged lymph nodes can be felt by gently pressing around the neck area, beginning at 10-week old. Enlarged lymph nodes can be an early sign reflecting the setting of lupus, with skin lesions forming as the animal ages.

Four groups of mice treated with mIgG, MH5A, 9F9 or 20E4, respectively, were examined for the sizes of their lymph nodes at 12, 14, and 15 weeks of age, respectively. Results are show in FIG. 21A, with data bars for each week from left to right being mIgG treatment group, MH5A treatment group, 9F9 treatment group, and 20E4 treatment group. Sizes of the lymph nodes were categorized into 5 numeric values: 0 (impalpable), 1 (mung bean size), 2 (pea size), 3 (peanut size), and 4 (walnut size). FIG. 21A shows that compared to the control group (mIgG1 treatment), MH5A and 9F9 significantly reduced the sizes of lymph nodes in mice, and 20E4 slight reduced the sizes of lymph nodes. FIG. 21B shows exemplary sizes of lymph nodes removed from the neck area of mice used in the experiment.

### B. Anti-VISTA Constructs in Reducing Serum Levels of Anti-nuclear Immunoglobulins

MRL-lpr mice develop systemic autoimmune symptoms, including spontaneous generation of autoantibodies (anti-nuclear antibodies, ANA) against cell nuclei. ANA test has been used in humans to diagnose lupus; a positive ANA test indicates that the immune system has launched a misdirected attack on a human's own tissue.

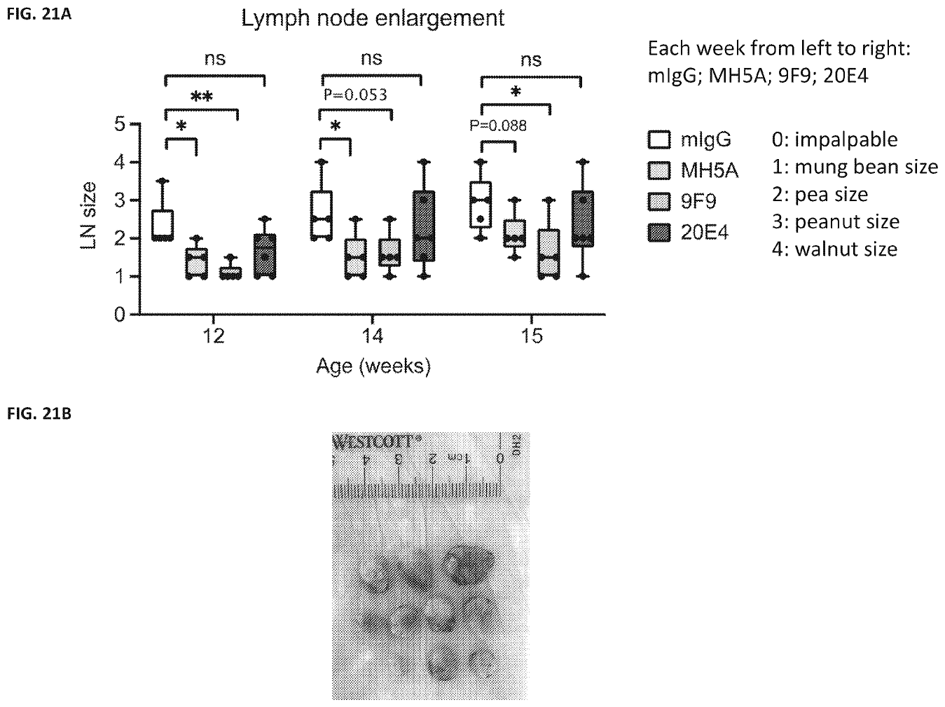

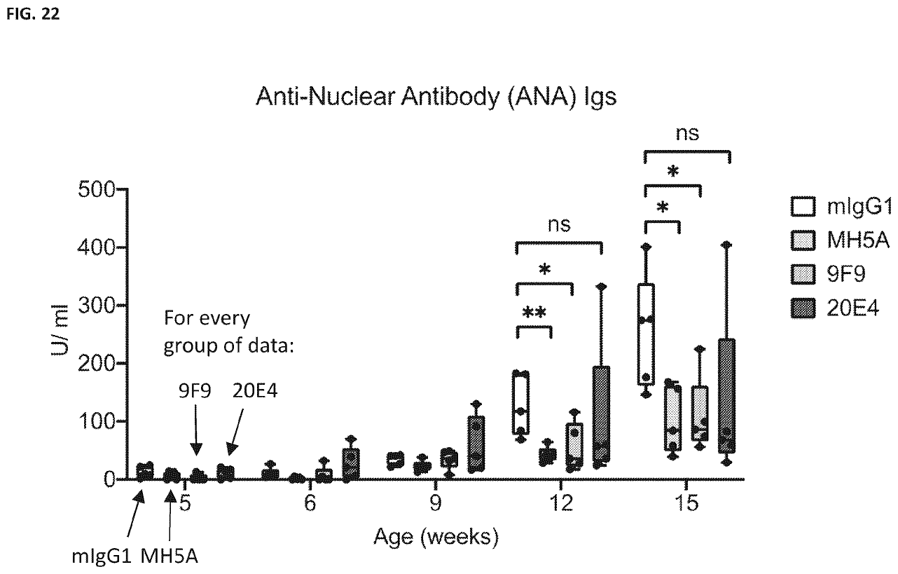

The serum ANA levels in mice treated with mIgG 1 , MHSA, 9F9, or 20E4 were determined at weeks 5, 6, 9, 12, and 15, respectively. Results are shown in FIG. 22. Data bars in FIG. 22 for each week from left to right being mIgG treatment group, MHSA treatment group, 9F9 treatment group, and 20E4 treatment group. MHSA and 9F9 significantly reduced the level of ANA in serum compared to mIgG1, and 20E4 mildly reduced serum level of ANA in mice by week 15.

### C. Anti-VISTA Constructs in Reducing Serum Levels of Anti-dsDNA Immunoglobulins

In the clinic, a high level of anti-dsDNA antibodies in the blood is strongly associated with lupus and is often significantly increased during or just prior to a flare-up. A positive anti-dsDNA antibody test in association with other clinical signs and symptoms associated with lupus is used for diagnosis of lupus.

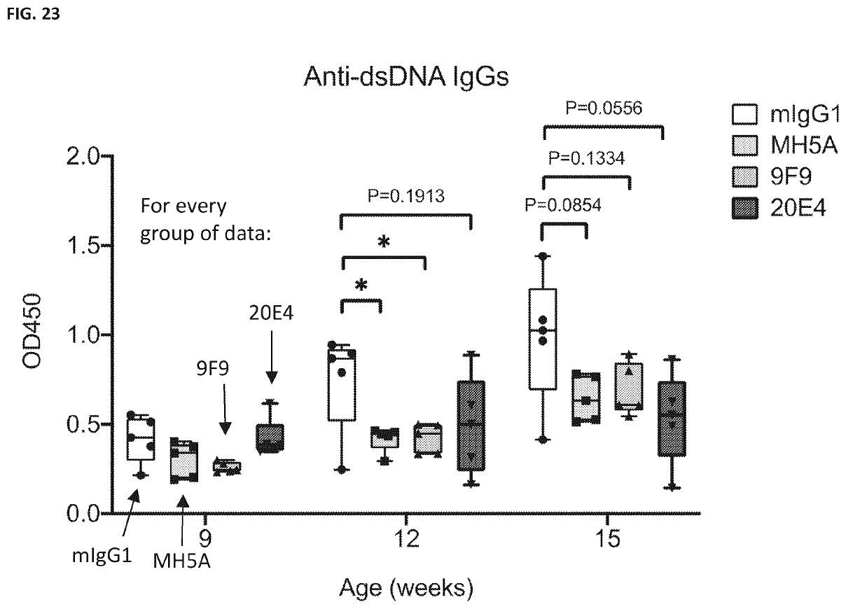

The serum levels of anti-dsDNA antibodies in mice treated with mIgG1, MH5A, 9F9, or 20E4 were determined at weeks 9, 12, and 15, respectively. Results are shown in FIG. 23. All anti-VISTA constructs tested (MH5A, 9F9, and 20E4) were able to reduce the anti-dsDNA antibodies in mouse serum at week 12, and the levels remained low 3 weeks post-treatment at week 15.

### D. Anti-VISTA Constructs in Reducing Serum Levels of IFNα

An example of an important cytokine involved in lupus etiology and pathogenesis is interferon alpha. IFNα is an important protein in immune regulation. IFNα is a pleiotropic cytokine that can affect multiple cell types involved in lupus. Increased serum levels of IFNα and altered expression of several genes in the interferon pathway are associated with risk for lupus, suggesting a role for this pathway in etiology.

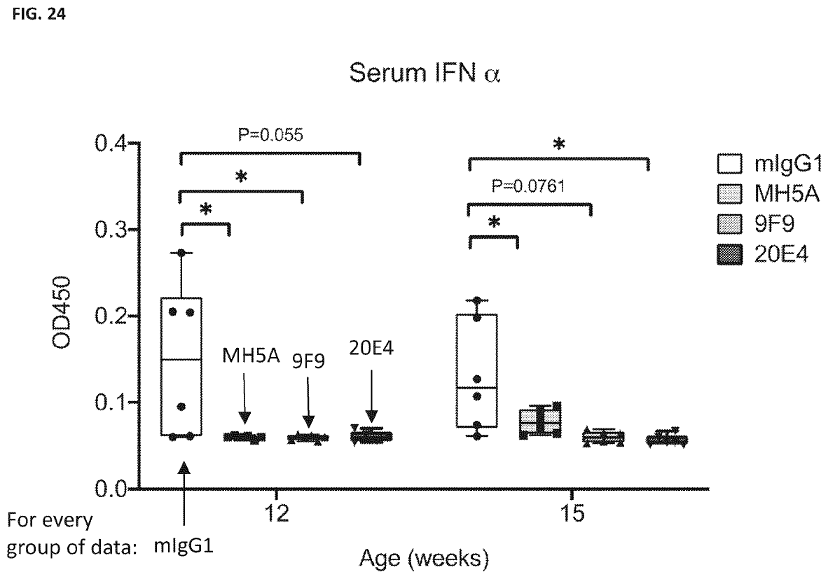

The serum levels of IFNα in mice treated with mIgG1, MH5A, 9F9, or 20E4 were determined at weeks 12 and 15. It is evidently shown in FIG. 24 that all 3 anti-VISTA constructs significantly reduced serum level of IFNα at week 12 and remained significantly lower at week 15 in comparison with the mIgG1 (control).

### E. Anti-VISTA Constructs in Reducing Protein Levels in Urine

MRL-lpr mice used herein spontaneously develop lupus nephritis. Protein levels in urine can reflect disease pathogenesis of the kidney. In the clinic, besides blood tests, urine tests including urine protein levels are used to diagnose and monitor the effects of lupus on the kidneys.

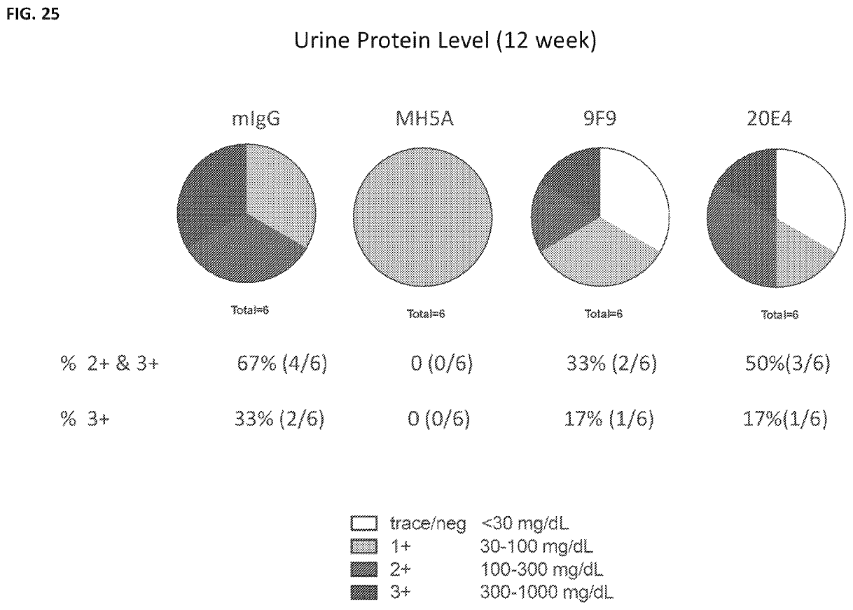

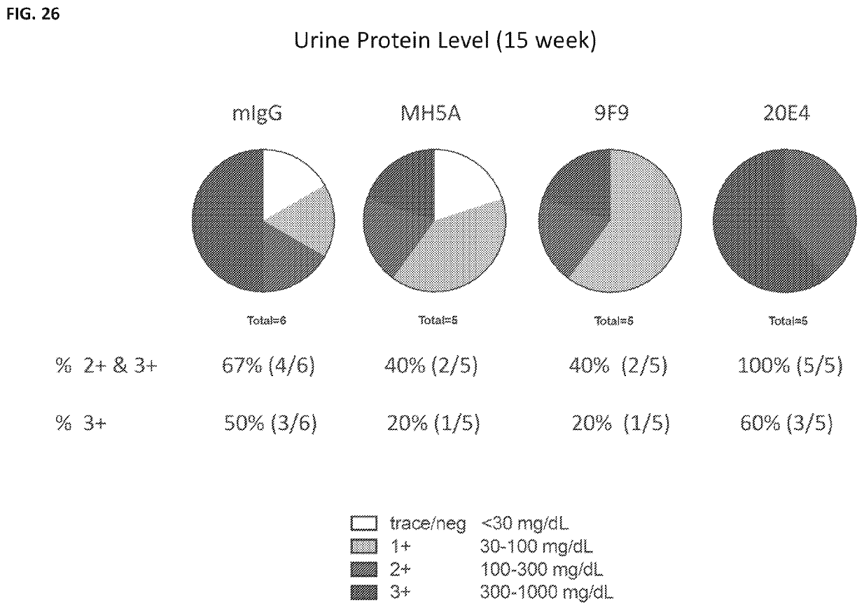

Protein levels in urine were measured at week 12 and 15 for mice treated with mIgG1, MH5A, 9F9, or 20E4. Each group had 6 mice. Pie charts in FIGS. 25 and 26 demonstrate the distribution of mice in different urine protein level categories in each treatment group. The percentage values under the charts show the percentages of mice in each treatment group with protein levels >100 mg/dL or >300 mg/dL. All treatment groups with anti-VISTA constructs had fewer mice in groups 2+(100-300 mg/dL protein) and 3+(300-1000 mg/dL protein) compared to the mIgG group at week 12. MH5A and 9F9 showed prolonged effects on maintaining reduced urine protein levels at week 15.

### F. Anti-VISTA Constructs in Reducing Skin Lupus Lesions

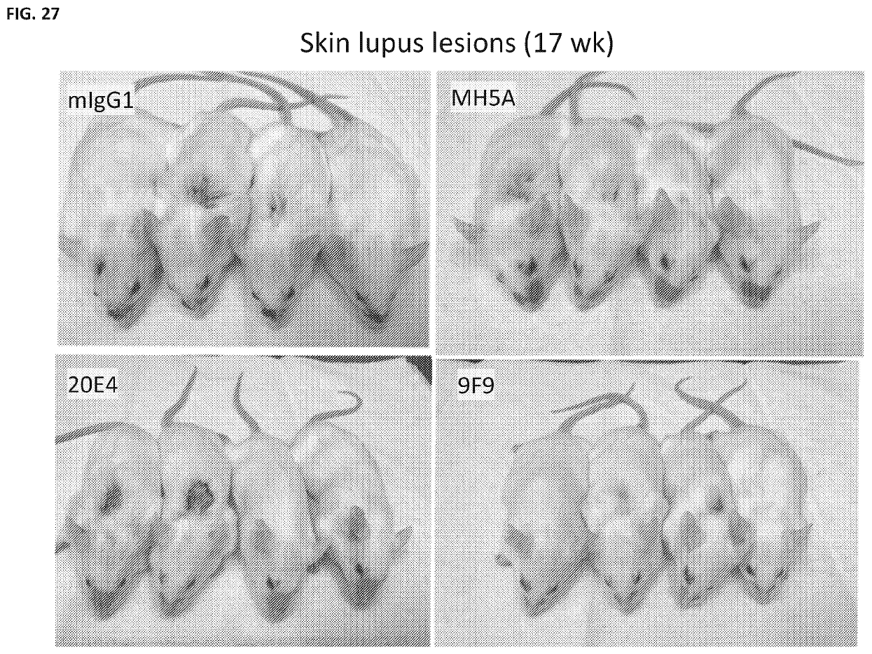

The protective effect of the anti-VISTA constructs against skin lupus lesions in mice was investigated at week 17. As shown in FIG. 27, mice treated with MH5A and 9F9 had reduced skin lesions compared to mice treated with mIgG1.

### G. Conclusions

MH5A, 9F9 and 20E4 were tested in MRL-lpr mice for their therapeutic effects on lupus including systemic lupus erythematosus (SLE). Lymph node enlargement, autoantibody and cytokine levels in serum, urine protein levels, and skin appearance were used to evaluate the severity of disease in each group after treatment. 9F9 and MH5A demonstrated promising pre-clinical effects in the MRL-lpr mouse model and significantly reduced lymph node enlargement, serum anti-nuclear and anti-dsDNA autoantibody levels, serum IFNα levels and urine protein levels in MRL-lpr mice. 20E4 also showed protective effects in decreasing serum anti-dsDNA and IFNα levels compared to mIgG1 (control).

### Example 17. Efficacy Study of Anti-VISTA Antibodies in the Graft vs Host (GvHD) Mouse Model

The goal of this study is to evaluate the efficacy of anti-VISTA antibodies 9F9 and 20E4 in a mouse model of graft vs host disease (GvHD).

The efficacy of the study was evaluated by measuring body weight and by visual evaluation of denudation of the skin of the mice. Throughout the study, the overall activity of the mice was evaluated. The level of human CD45+ cell engraftment, which indicates the level of GvHD development in the blood was also measured. See e.g., Ali et al., PLoS One. 2012; 7(8): e44219.

NOD-scid IL2rg null (NSG) mice were used for this study. On Day 0, all mice were injected with 10 million human PBMCs by intravenous (IV) injection and divided into three groups. Group 1 received a mouse IgG isotype control (0.5 mg/mouse) treatment on Day 0. Groups 2 and 3 were treated with 9F9 (0.5 mg/mouse) or 20E4 (0.5 mg/mouse) respectively by intraperitoneal (i.p.) injection. Body weight was collected weekly. A second dose of test articles was given at 2 weeks. Blood samples were taken on week 2 and week 5 for flow cytometry analysis. Mice were sacrificed on Day 37.

Measurements collected as raw data were analyzed using GraphPad software Prism. P<0.05 was considered statistically significant.

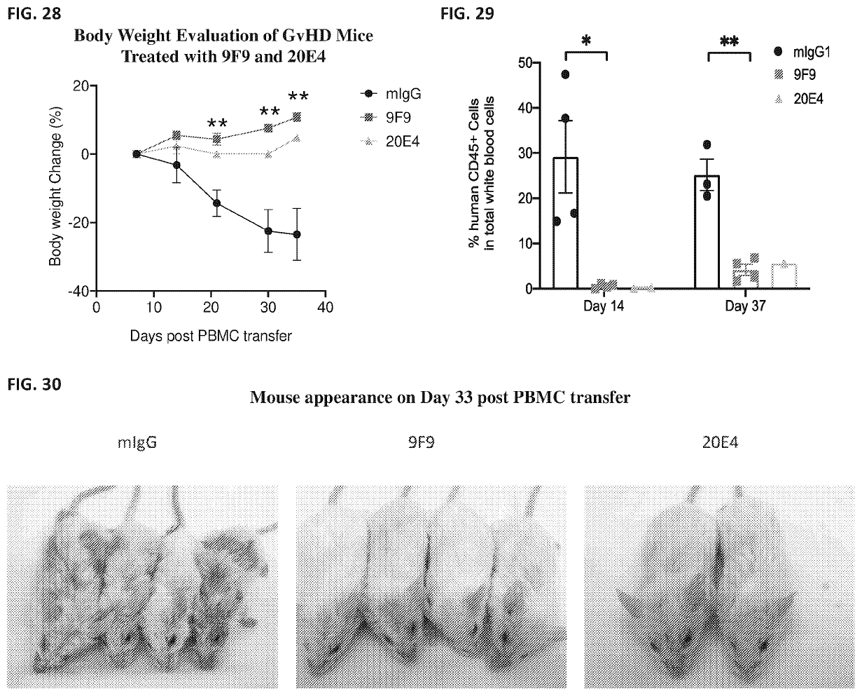

Graft vs host disease results in a loss in body weight as the animals become lethargic and have reduced activity as the disease progresses. FIG. 28 shows that treatment of the mice with anti-VISTA antibodies 9F9 and 20E4 prevent loss of body weight for the duration of the study. The change in body weight was statistically different in the 9F9 and 20E4 treated groups compared to the isotype control group. A reduction in activity was observed for three out of four of the isotype control treated mice. No activity change was observed for either of the groups treated with 9F9 or 20E4 antibodies.

Mice suffering from GvHD appear with skin denudation over time as the illness progresses. On Day 33, the mice were photographed to capture the level of skin denudation observed in the different groups. FIG. 30 shows the skin denudation in the mice injected with the isotype control versus those treated with the anti-VISTA antibodies 9F9 or 20E4.

Serum was collected on Day 14 and Day 37 after the PBMC transfer. Cells were collected and stained for human and mouse CD45 for flow cytometry analysis. FIG. 29 shows the percent of human CD45+ cells in the different groups. Each symbol represents an individual mouse in the group. There was a statistically significant reduction in human CD45+cells in the mice treated with either 9F9 or 20E4 antibodies compared to the control.

SEQUENCE TABLE

| SEQ | | |
|---|---|---|
| ID | | |
| NO. | Description | Amino Acid Sequence |
| 1. | 9F9 HC- | GYWMQ |
| | CDR1 | |
| | (Kabat) | |
| 2. | 9F9 HC- | AIYPGDGDTRYSQKFKG |
| | CDR2 | |
| | (Kabat) | |
| 3. | 9F9 HC- | RDYGAWFAY |
| | CDR3 | |
| | (Kabat) | |
| 4. | 9F9 LC- | KASQDINSYLS |
| | CDR1 | |
| | (Kabat) | |
| 5. | 9F9 LC- | RANRLVD |
| | CDR2 | |
| | (Kabat) | |
| 6. | 9F9 LC- | LQYDEFPYT |
| | CDR3 | |
| | (Kabat) | |
| 7. | 9F9 VH | QVQFQQSGAELARPGASVKLSCKASGYTFTGYWMQWVKQRP |
| | Amino Acid | GQGLEWIGAIYPGDGDTRYSQKFKGKVTLTADKSSSTAYMQLS |
| | Sequence | SLASEDSAVYYCARRDYGAWFAYWGQGTLVTVSA |
| 8. | 9F9 VL | DIKVTQSPSSMYASLGERVTITCKASQDINSYLSWFQQKPGKSP |
| | Amino Acid | KTLIYRANRLVDGVPSRFSGSGSGQDYSLTISSLDYEDMGIYYC |
| | Sequence | LQYDEFPYTFGGGTKLEIK |
| 9. | 20E4 HC- | SSWVE |
| | CDR1 | |
| | (Kabat) | |
| 10. | 20E4 HC- | EIFPGSGSTHYNEKFKG |
| | CDR2 | |
| | (Kabat) | |
| 11. | 20E4 HC- | RPPGWFFDV |
| | CDR3 | |
| | (Kabat) | |
| 12. | 20E4 LC- | KASQDVSTDVA |
| | CDR1 | |
| | (Kabat) | |
| 13. | 20E4 LC- | SASYRYT |
| | CDR2 | |
| | (Kabat) | |
| 14. | 20E4 LC- | QQHYSTPWT |
| | CDR3 | |
| | (Kabat) | |
| 15. | 20E4 VH | QVQLQQSGAELMKPGASVKISCKATGYTFSSSWVEWVRQRPG |
| | Amino Acid | HGLEWIGEIFPGSGSTHYNEKFKGKATFTADTSSNTAYLQLSSL |
| | Sequence | TSDDSAVYYCARRPPGWFFDVWGAGTTVTVSS |
| 16. | 20E4 VL | DIVMTQSHKFMSTSVGDRVSITCKASQDVSTDVAWYQQKPGQ |
| | Amino Acid | SPKLLIYSASYRYTGVPDRFTGSGSGTDFTFTISSVQAEDLAVYY |
| | Sequence | CQQHYSTPWTFGGGTKLEIK |
| 17. | 16A1 HC- | SSWIE |
| | CDR1 | |
| | (Kabat) | |
| 18. | 16A1 HC- | EIFPGSGSLNFNEKFKG |
| | CDR2 | |
| | (Kabat) | |
| 19. | 16A1 HC- | RPPGWFFDV |
| | CDR3 | |
| | (Kabat) | |
| 20. | 16A1 LC- | KASQDVTTDVA |
| | CDR1 | |
| | (Kabat) | |
| 21. | 16A1 LC- | SASYRYT |
| | CDR2 | |
| | (Kabat) | |
| 22. | 16A1 LC- | QQHYSSPWT |
| | CDR3 | |
| | (Kabat) | |
| 23. | 16A1 VH | QVQLQQSGAELMKPGASVKISCKATGYTFSSSWIEWVKQRPGH |
| | Amino Acid | GLEWIGEIFPGSGSLNFNEKFKGKATFTADTSSNTAYLQLSSLTS |
| | Sequence | DDSAVYYCARRPPGWFFDVWGAGTTVTVSS |
| 24. | 16A1 VL | DIVMTQSHKFMSTSVGDRVSITCKASQDVTTDVAWYQQKPGQ |
| | Amino Acid | SPKLLIYSASYRYTGVPDRFTGSGSGTDFTFTISSVQAEDLTVYY |
| | Sequence | CQQHYSSPWTFGGGTKLEIK |
| 25. | 17E9 HC- | SCWIE |
| | CDR1 | |
| | (Kabat) | |
| 26. | 17E9 HC- | EILPGSDYTNYNEKFKD |
| | CDR2 | |
| | (Kabat) | |
| 27. | 17E9 HC- | RPPGWYFDV |
| | CDR3 | |
| | (Kabat) | |
| 28. | 17E9 LC- | KASQDVSTDIA |
| | CDR1 | |
| | (Kabat) | |
| 29. | 17E9 LC- | SASYRFT |
| | CDR2 | |
| | (Kabat) | |
| 30. | 17E9 LC- | QHQYSTPWT |
| | CDR3 | |
| | (Kabat) | |
| 31. | 17E9 VH | QVQLQQSGAELMKPGASVKISCTATGYTFSSCWIEWVKQRPGH |
| | Amino Acid | GLEWLGEILPGSDYTNYNEKFKDKATFTADTSSDTAYMQLSSL |
| | Sequence | TSEDSAVYYCARRPPGWYFDVWGAGTAVTVSS |
| 32. | 17E9 VL | DIVMTQSHKFMSTSVGDRVSITCKASQDVSTDIAWYQQKPGQS |
| | Amino Acid | PKLLIYSASYRFTGVPDRFTGSGSGTDFTFTISSVQAEDLAVYYC |
| | Sequence | QHQYSTPWTFGGGTKLDIK |
| 33. | 15D11 HC- | DTFIH |
| | CDR1 | |
| | (Kabat) | |
| 34. | 15D11 HC- | RIDPANGNSKYDPKFQG |
| | CDR2 | |
| | (Kabat) | |
| 35. | 15D11 HC- | WPSNWEAMDY |
| | CDR3 | |
| | (Kabat) | |
| 36. | 15D11 LC- | SASSSVSYMY |
| | CDR1 | |
| | (Kabat) | |
| 37. | 15D11 LC- | DTSNLAS |
| | CDR2 | |
| | (Kabat) | |
| 38. | 15D11 LC- | QQWISYPLT |
| | CDR3 | |
| | (Kabat) | |
| 39. | 15D11 VH | EVQLQQSGSELVKAGASVKLSCTASGFKIKDTFIHWVKQRPEQ |
| | Amino Acid | GLDWIGRIDPANGNSKYDPKFQGKATITADTSSNTAYLQLSSLT |
| | Sequence | SEDTAVYYCARWPSNWEAMDYWGQGTSVTVSS |
| 40. | 15D11 VL | QIVLTQSPAIMSASPGEKVTMTCSASSSVSYMYWYQQKPGSSPR |
| | Amino Acid | LLIYDTSNLASGVPFRFSGSGSGTSYSLTISRMEAEDAATYYCQ |
| | Sequence | QWISYPLTFGTGTKLELK |
| 41. | HC-CDR1 | SSWX₁E |
| | consensus | X₁ = V or I |
| | sequence | |
| | based on | |
| | 20E4 and | |
| | 16A1 | |
| 42. | HC-CDR2 | EIFPGSGSX₂X₃X₄NEKFKG |
| | consensus | X₂ = T or L, X₃ = H or N, X₄ = Y or F |
| | sequence | |
| | based on | |
| | 20E4 and | |
| | 16A1 | |
| 43. | HC-CDR1 | SX₁WX₂E |
| | consensus | X₁ = S or C, X₂ = V or I |
| | sequence | |
| | based on | |
| | 20E4 and | |
| | 17E9 | |
| 44. | HC-CDR2 | EIX₃PGSX₄X₅TX₆YNEKFKX₇ |
| | consensus | X₃ = F or L, X₄ = G or D, X₅ = Y or S, X₆ = H or N, |
| | sequence | X₇ = G or D |
| | based on | |
| | 20E4 and | |
| | 17E9 | |
| 45. | HC-CDR3 | RPPGWX₈FDV |
| | consensus | X₈ = F or Y |
| | sequence | |
| | based on | |
| | 20E4 and | |
| | 17E9 | |
| 46. | LC-CDR1 | KASQDVX₁TDVA |
| | consensus | X₁ = S or T |
| | sequence | |
| | based on | |
| | 20E4 and | |
| | 16A1 | |
| 47. | LC-CDR3 | QQHYSX₂PWT |
| | consensus | X₂ = S or T |
| | sequence | |
| | based on | |
| | 20E4 and | |
| | 16A1 | |
| 48. | LC-CDR1 | KASQDVX₁TDX₂A |
| | consensus | X₁ = S or T, X₂ = V or I |
| | sequence | |
| | Based on | |
| | 20E4, 16A1 | |
| | and 17E9 | |
| 49. | LC-CDR2 | SASYRX₃T |
| | consensus | X₃ = Y or F |
| | sequence | |
| | Based on | |
| | 20E4, 16A1 | |
| | and 17E9 | |
| 50. | LC-CDR3 | QX₄X₅YSX₆PWT |
| | consensus | X₄ = Q or H, X₅ = Q or H, X₆ = S or T |
| | sequence | |
| | Based on | |
| | 20E4, 16A1 | |
| | and 17E9 | |
| 51. | HC-CDR2 | EIFPGSGSX₂X₃X₄NEKFKG |
| | consensus | X₂X₃X₄ = THY or LNF |
| | sequence | |
| | based on | |
| | 20E4 and | |
| | 16A1 | |
| 52. | HC-CDR2 | EIX₃PGSX₄X₅TX₆YNEKFKX₇ |
| | consensus | X₃ = F or L, X₄X₅ = DY or GS, X₆ = H or N, |
| | sequence | X₇ = G or D |
| | based on | |
| | 20E4 and | |
| | 17E9 | |
| 53. | LC-CDR3 | QX₄X₅YSX₆PWT |
| | consensus | X₄X₅ = QH or HQ, X₆ = S or T |
| | sequence | |
| | Based on | |
| | 20E4, 16A1 | |
| | and 17E9 | |
| 54. | LC-CDR1 | KASQDVSTDX₁A |
| | Consensus | X₁ = V or I |
| | sequence | |
| | Based on | |
| | 20E4 and | |
| | 17E9 | |
| 55. | LC-CDR2 | SASYRX₂T |
| | Consensus | X₂ = Y or F |
| | sequence | |
| | Based on | |
| | 20E4 and | |
| | 17E9 | |
| 56. | LC-CDR3 | QX₃X₄YSTPWT |
| | Consensus | X₄ = Q or H, X₅ = Q or H |
| | sequence | |
| | Based on | |
| | 20E4 and | |
| | 17E9 | |
| 57. | LC-CDR3 | QX₃X₄YSTPWT |
| | Consensus | X₄X₅ = QH or HQ |
| | sequence | |
| | Based on | |
| | 20E4 and | |
| | 17E9 | |
| 58. | LC-CDR2 | EIX₃PGSX₄X₅X₆X₇X₈NEKFKX₉ |
| | Consensus | X₃ = F or L, X₄X₅X₆X₇X₈ = GSTHY, GSLNF, |
| | sequence | or DYTNY, X₉ = F or L |
| | based on | (SEQ ID NO: 58) |
| | 20E4, 16A1 | |
| | and 17E9 | |
| 59. | Human | MGVPTALEAGSWRWGSLLFALFLAASLGPVAAFKVATPYSLY |
| | VISTA | VCPEGQNVTLTCRLLGPVDKGHDVTFYKTWYRSSRGEVQTCSE |
| | sequence | RRPIRNLTFQDLHLHHGGHQAANTSHDLAQRHGLESASDHHGN |
| | | FSITMRNLTLLDSGLYCCLVVEIRHHHSEHRVHGAMELQVQTG |
| | | KDAPSNCVVYPSSSQDSENITAAALATGACIVGILCLPLILLLVY |
| | | KQRQAASNRRAQELVRMDSNIQGIENPGFEASPPAQGIPEAKVR |
| | | HPLSYVAQRQPSESGRHLLSEPSTPLSPPGPGDVFFPSLDPVPDSP |
| | | NFEVI |

1. An anti-VISTA construct comprising an antibody moiety comprising a heavy chain variable region (V_{H}) and a light chain variable region (V_{L}), wherein the antibody moiety competes for a binding epitope of VISTA with an antibody or antibody fragment comprising a second heavy chain variable region (V_{H-2}) and a second light chain variable region (V_{L-2}), wherein:
a) the VH-2 comprising the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6;
b) the VH-2 comprises the HC-CDR1 comprising the amino acid sequence of SEQ ID NO: 9, the HC-CDR2 comprising the amino acid sequence of SEQ ID NO: 10, and the HC-CDR3 comprising the amino acid sequence of SEQ ID NO: 11, and the V_{L-2} comprises the LC-CDR1 comprising the amino acid sequence of SEQ ID NO: 12, the LC-CDR2 comprising the amino acid sequence of SEQ ID NO: 13, and the LC-CDR3 comprising the amino acid sequence of SEQ ID NO: 14;
c) the V_{H-2} comprises the HC-CDR1 comprising the amino acid sequence of SEQ ID NO: 17, the HC-CDR2 comprising the amino acid sequence of SEQ ID NO: 18, and the HC-CDR3 comprising the amino acid sequence of SEQ ID NO: 19, and the V_{L-2} comprises the LC-CDR1 comprising the amino acid sequence of SEQ ID NO: 20, the LC-CDR2 comprising the amino acid sequence of SEQ ID NO: 21, and the LC-CDR3 comprising the amino acid sequence of SEQ ID NO: 22;
d) the VH-2 comprises the HC-CDR1 comprising the amino acid sequence of SEQ ID NO: 25, the HC-CDR2 comprising the amino acid sequence of SEQ ID NO: 26, and the HC-CDR3 comprising the amino acid sequence of SEQ ID NO: 27, and the V_{L-2} comprises the LC-CDR1 comprising the amino acid sequence of SEQ ID NO: 28, the LC-CDR2 comprising the amino acid sequence of SEQ ID NO: 29, and the LC-CDR3 comprising the amino acid sequence of SEQ ID NO: 30; or
e) the VH-2 comprises the HC-CDR1 comprising the amino acid sequence of SEQ ID NO: 33, the HC-CDR2 comprising the amino acid sequence of SEQ ID NO: 34, and the HC-CDR3 comprising the amino acid sequence of SEQ ID NO: 35, and the V_{L-2} comprises the LC-CDR1 comprising the amino acid sequence of SEQ ID NO: 36, the LC-CDR2 comprising the amino acid sequence of SEQ ID NO: 37, and the LC-CDR3 comprising the amino acid sequence of SEQ ID NO: 38.

2. The anti-VISTA construct of claim 1, wherein:
a) the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising 5, 4, 3, 2, or 1 amino acid substitutions in the HC-CDRs; and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising 5, 4, 3, 2, or 1 amino acid substitutions in the LC-CDRs;
b) the V_{H} comprises i) the HC-CDR1 comprising the amino acid sequence of SEQ ID NO: 9, ii) the HC-CDR2 comprising the amino acid sequence of SEQ ID NO: 10, and iii) the HC-CDR3 comprising the amino acid sequence of SEQ ID NO: 11, or a variant thereof comprising 5, 4, 3, 2, or 1 amino acid substitutions in the HC-CDRs; and the V_{L} comprises i) the LC-CDR1 comprising the amino acid sequence of SEQ ID NO: 12, ii) the LC-CDR2 comprising the amino acid sequence of SEQ ID NO: 13, and iii) the LC-CDR3 comprising the amino acid sequence of SEQ ID NO: 14, or a variant thereof comprising 5, 4, 3, 2, or 1 amino acid substitutions in the LC-CDRs;
c) the V_{H} comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19, or a variant thereof comprising 5, 4, 3, 2, or 1 amino acid substitutions in the HC-CDRs; and the V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22, or a variant thereof comprising 5, 4, 3, 2, or 1 amino acid substitutions in the LC-CDRs;
d) the V_{H} comprises i) the HC-CDR1 comprising the amino acid sequence of SEQ ID NO: 25, ii) the HC-CDR2 comprising the amino acid sequence of SEQ ID NO: 26, and iii) the HC-CDR3 comprising the amino acid sequence of SEQ ID NO: 27, or a variant thereof comprising 5, 4, 3, 2, or 1 amino acid substitutions in the HC-CDRs; and the V_{L} comprises i) the LC-CDR1 comprising the amino acid sequence of SEQ ID NO: 28, ii) the LC-CDR2 comprising the amino acid sequence of SEQ ID NO: 29, and iii) the LC-CDR3 comprising the amino acid sequence of SEQ ID NO: 30, or a variant thereof comprising 5, 4, 3, 2, or 1 amino acid substitutions in the LC-CDRs;
e) the V_{H} comprises i) the HC-CDR1 comprising the amino acid sequence of SEQ ID NO: 33, ii) the HC-CDR2 comprising the amino acid sequence of SEQ ID NO: 34, and iii) the HC-CDR3 comprising the amino acid sequence of SEQ ID NO: 35, or a variant thereof comprising 5, 4, 3, 2, or 1 amino acid substitutions in the HC-CDRs; and the V_{L} comprises i) the LC-CDR1 comprising the amino acid sequence of SEQ ID NO: 36, ii) the LC-CDR2 comprising the amino acid sequence of SEQ ID NO: 37, and iii) the LC-CDR3 comprising the amino acid sequence of SEQ ID NO: 38, or a variant thereof comprising 5, 4, 3, 2, or 1 amino acid substitutions in the LC-CDRs;
f) the V_{H} comprises i) the HC-CDR1 comprising the amino acid sequence of SEQ ID NO: 41, ii) the HC-CDR2 comprising the amino acid sequence of SEQ ID NO: 42 or 51, and iii) the HC-CDR3 comprising the amino acid sequence of SEQ ID NO: 11; and the V L comprises i) the LC-CDR1 comprising the amino acid sequence of SEQ ID NO: 46, ii) the LC-CDR2 comprising the amino acid sequence of SEQ ID NO: 13, and iii) the LC-CDR3 comprising the amino acid sequence of SEQ ID NO: 47;
h) the V_{H} comprises i) the HC-CDR1 comprising the amino acid sequence of SEQ ID NO: 43, ii) the HC-CDR2 comprising the amino acid sequence of SEQ ID NO: 44 or 52, and iii) the HC-CDR3 comprising the amino acid sequence of SEQ ID NO: 45; and the VL comprises i) the LC-CDR1 comprising the amino acid sequence of SEQ ID NO: 54, ii) the LC-CDR2 comprising the amino acid sequence of SEQ ID NO: 55, and iii) the LC-CDR3 comprising the amino acid sequence of SEQ ID NO: 56 or 57; or
i) the V_{H} comprises i) the HC-CDR1 comprising the amino acid sequence of SEQ ID NO: 41 or 43, ii) the HC-CDR2 comprising the amino acid sequence of any of SEQ ID NO:58, and iii) the HC-CDR3 comprising the amino acid sequence of SEQ ID NO: 11 or 45;
and the V_{L} comprises i) the LC-CDR1 comprising the amino acid sequence of SEQ ID NO:
48, ii) the LC-CDR2 comprising the amino acid sequence of SEQ ID NO: 49, and iii) the LC-CDR3 comprising the amino acid sequence of SEQ ID NO: 50 or 53.

3. The anti-VISTA construct of claim 2, wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3; and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6.

4. The anti-VISTA construct of claims 2, wherein the V_{H} comprises i) the HC-CDR1 comprising the amino acid sequence of SEQ ID NO: 9, ii) the HC-CDR2 comprising the amino acid sequence of SEQ ID NO: 10, and iii) the HC-CDR3 comprising the amino acid sequence of SEQ ID NO: 11; and the V_{L} comprises i) the LC-CDR1 comprising the amino acid sequence of SEQ ID NO: 12, ii) the LC-CDR2 comprising the amino acid sequence of SEQ ID NO: 13, and iii) the LC-CDR3 comprising the amino acid sequence of SEQ ID NO: 14.

5. The anti-VISTA construct of claim 3, wherein the VH comprises i) the HC-CDR1 comprising the amino acid sequence of SEQ ID NO: 17, ii) the HC-CDR2 comprising the amino acid sequence of SEQ ID NO: 18, and iii) the HC-CDR3 comprising the amino acid sequence of SEQ ID NO: 19; and the V_{L} comprises i) the LC-CDR1 comprising the amino acid sequence of SEQ ID NO: 20, ii) the LC-CDR2 comprising the amino acid sequence of SEQ ID NO: 21, and iii) the LC-CDR3 comprising the amino acid sequence of SEQ ID NO: 22.

6. The anti-VISTA construct of claim 3, wherein the V_{H} comprises i) the HC-CDR1 comprising the amino acid sequence of SEQ ID NO: 25, ii) the HC-CDR2 comprising the amino acid sequence of SEQ ID NO: 26, and iii) the HC-CDR3 comprising the amino acid sequence of SEQ ID NO: 27; and the V_{L} comprises i) the LC-CDR1 comprising the amino acid sequence of SEQ ID NO: 28, ii) the LC-CDR2 comprising the amino acid sequence of SEQ ID NO: 29, and iii) the LC-CDR3 comprising the amino acid sequence of SEQ ID NO: 30.

7. An anti-VISTA construct comprising an antibody moiety that specifically binds to VISTA, comprising:
a) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 7, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 8;
b) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 15, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 16;
c) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 23, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 24;
d) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 31, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 32; or
e) a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 39, and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 40.

8. The anti-VISTA construct of any one of claims 1-7, wherein the V_{H} comprises an amino acid sequence of any one of SEQ ID NOs: 7, 15, 23, 31, and 39, or a variant comprising an amino acid sequence having at least about 80% sequence identity; and/or wherein the V_{L} comprises an amino acid sequence of any one of SEQ ID NOs: 8, 16, 24, 32 and 40, or a variant comprising an amino acid sequence having at least about 80% sequence identity.

9. The anti-VISTA construct of claim 8, wherein:
a) the V_{H} comprises an amino acid sequence of SEQ ID NO: 7, or a variant comprising an amino acid sequence having at least about 80% sequence identity; and the V_{L} comprises an amino acid sequence of SEQ ID NO: 8, or a variant comprising an amino acid sequence having at least about 80% sequence identity,
b) the V_{H} comprises an amino acid sequence of SEQ ID NO: 15, or a variant comprising an amino acid sequence having at least about 80% sequence identity; and the V_{L} comprises an amino acid sequence of SEQ ID NO: 16, or a variant comprising an amino acid sequence having at least about 80% sequence identity,
c) the V_{H} comprises an amino acid sequence of SEQ ID NO: 23, or a variant comprising an amino acid sequence having at least about 80% sequence identity; and the V_{L} comprises an amino acid sequence of SEQ ID NO: 24, or a variant comprising an amino acid sequence having at least about 80% sequence identity,
d) the V_{H} comprises an amino acid sequence of SEQ ID NO: 31, or a variant comprising an amino acid sequence having at least about 80% sequence identity; and the V_{L} comprises an amino acid sequence of SEQ ID NO: 32, or a variant comprising an amino acid sequence having at least about 80% sequence identity, or
e) the V_{H} comprises an amino acid sequence of SEQ ID NO: 39, or a variant comprising an amino acid sequence having at least about 80% sequence identity; and the V_{L} comprises an amino acid sequence of SEQ ID NO: 40, or a variant comprising an amino acid sequence having at least about 80% sequence identity.

10. The anti-VISTA construct of any one of claims 1-9, wherein the antibody moiety is an antibody or antigen-binding fragment thereof selected from the group consisting of a full-length antibody, a bispecific antibody, a single-chain Fv (scFv) fragment, a Fab fragment, a Fab' fragment, a F(ab')2, an Fv fragment, a disulfide stabilized Fv fragment (dsFv), a (dsFv)2, a Fv-Fc fusion, a scFv-Fc fusion, a scFv-Fv fusion, a diabody, a tribody, and a tetrabody.

11. The anti-VISTA construct of claim 10, wherein the antibody moiety is a full-length antibody.

12. The anti-VISTA construct of any one of claims 1-11, wherein the antibody moiety has an Fc fragment is selected from the group consisting of Fc fragments form IgG, IgA, IgD, IgE, IgM, and combinations and hybrids thereof.

13. The anti-VISTA construct of claim 12, wherein the Fc fragment is selected from the group consisting of Fc fragments from IgG1, IgG2, IgG3, IgG4, and combinations and hybrids thereof.

14. The anti-VISTA construct of claim 12 or claim 13, wherein the Fc fragment has a reduced effector function as compared to the corresponding wildtype Fc fragment.

15. The anti-VISTA construct of any one of claims 12-14, wherein the Fc fragment has an extended half-life as compared to the corresponding wildtype Fc fragment.

16. The anti-VISTA construct of any one of claims 1-15, wherein the antibody moiety of the anti-VISTA construct activates the downstream signaling pathways of VISTA.

17. The anti-VISTA construct of any one of claims 1-16, wherein the anti-VISTA construct is an agonist antibody of VISTA.

18. The anti-VISTA construct of any one of claim 16, wherein the antibody moiety of the anti-VISTA construct activates or increases the downstream signaling pathways of VISTA by at least about 20%.

19. The anti-VISTA construct of any one of claims 1-18, wherein the VISTA is a human VISTA.

20. A pharmaceutical composition comprising the anti-VISTA construct of any one of claims 1-19, and a pharmaceutical acceptable carrier.

21. An isolated nucleic acid encoding the anti-VISTA construct of any one of claims 1-20.

22. A vector comprising the isolated nucleic acid of claim 21.

23. An isolated host cell comprising the isolated nucleic acid of claim 21, or the vector of claim 22.

24. An immunoconjugate comprising the anti-VISTA construct of any one of claims 1-19, linked to a therapeutic agent or a label.

25. A method of producing an anti-VISTA construct comprising:
a) culturing the isolated host cell of claim 23 under conditions effective to express the anti-VISTA construct; and
b) obtaining the expressed anti-VISTA construct from the host cell.

26. A method of treating a disease or condition in an individual, comprising administering to the individual an effective mount of the anti-VISTA construct of any one of claims 1-19, or the pharmaceutical composition of claim 20.

27. The method of claim 26, wherein the disease of condition is associated with a dysregulated immune system.

28. The method of claim 26 or claim 27, wherein the disease or condition is an auto-immune disease, inflammation, an infection, graft versus host disease (GvHD) or a condition associated with a transplant.

29. The method of claim 28, wherein the auto-immune disease is selected from cutaneous lupus, rheumatoid arthritis, psoriasis, an autoimmune intestinal disorder, systemic lupus erythematosus (SLE), discoid lupus erythematosus (DLE).

30. The method of any one of claims 26-29, wherein the anti-VISTA construct is administered intravenously or subcutaneously into the individual.

31. The method of any one of claims 26-30, wherein the anti-VISTA construct is administered at a dose of about 0.001 mg/kg to about 100 mg/kg.

32. The method of any one of claims 22-31, wherein the individual is a human.

33. A kit comprising any one of the anti-VISTA construct of claims 1-19.
